SEQUENCE LISTING (12 sequences)

SEQ ID NO: 1 - protein, 376 aa, Algoriphagus sanaruensis
MKKILFLSGTRADFGKIKSLIQVCSDDPNFEVHIFATGMHMHKQYGETVIEIEKCGFKNIYKFINHTSETSMDLTLAKTIDGLSSYVKENRPDLIIVHGDRVEALAGAIVGALNNILVGHIEGGEVSGTIDELIRHSVSKLSHIHFVSNELAKNRLIQMGELESQVYVIGSPDIDIMFSNSLPSLEVALGYYGIPFDRFAVLMFHPVTTEVNQIEQYSRLLVDALLDDSSNYIVIFPNNDMGSSKILQAYKSFEGHPRFKVFPSLRFEYFLSILKHAEFIIGNSSAGIREAPYYGIPSINIGSRQQNRALDPSIIHADFSGESLKSAINQAKTSGRTSPGRQFGSGNSYTLFHSIISQDEFWHIEHQKLFNDLSKN

SEQ ID NO: 2 - protein, 372 aa, Pseudoalteromonas luteoviolacea
MKKILFLTGTRADFGKLKSLIKKVESSQHEAYVFVTGMHMIEKYGSTVLEVEKSVESSIYKFINQNPHDTMDIVLSKTTAGLSDYVKGIKPDLIVVHGDRVEALAGALVGALNNILVAHIEGGEVSGTIDELIRHSVSKMAHIHFVCNDDARQRLLQLGECEDSIFVIGSPDLDIMNSATLPDIQLVKEHYELPFDNYSILLFHPVTTELDSLTQQAEALADAAIESGRNFVVIYPNNDHGNEMILSAFSKFENNAQFAVFPSVRFEYFLTLLKNADCMIGNSSAGVREAPFYGLPSIDIGSRQKNRSSAKSIVNVEPVKSDILDAFKKVESMKCDATREFGEGKSDEKFIDALNSNSLWKLPKQKMFIDRG

SEQ ID NO: 3 - protein, 373 aa, Campylobacter upsaliensis
  source 1..373: note = JV21
MKKIVFITGTRADFSKIKSLMLKVEKANDFELFIFATGMHMSKNFGLTVREIEKCGFKNIFKFINHDKYFQMDKALSATIDGFSRFIHELEPDLIVVHGDRIEPLAACMVGSLNNILVAHIEGGEISGTIDDSIRHAISKLAHIHLVNDQMAKKRLIQMGEAEDSIFIIGSPDLEILAHNKISLKLAKNYYDIPYENYALVMFHPVTTEFHTLKEQSDCLVEALKKSQKNYIVIYPNNDLGFEQILQSYEDLKKFKNFKFFPSLRFEYFITLLKNADFIIGNSSCILKEAVYLNIPGILVGSRQNGRKGRESVRCVETKMEVILQAIHTLQKPNNINKNKKLKILDSSKLFFKHLQNGDFFKIKQQKIFKDLK

SEQ ID NO: 4 - protein, 386 aa, Helicobacter felis
MKKHLVFVTNSRADYGKLKPLIQAIDALEKGKLDHPMAGKITYAIFVCGMHLLEKYGSTYIEIFKDGFEHVFLAKATPQEMDLALADTIKQFHHFVTLQRPDLIIFHGDRLETLACAIVGSFNSILSAHIEGGELSGTLDESIRHSVSKLAHLHFVCEERARQILLQMGERAENIFVIGSSDIDVMLGDLPSFPQVESRYSQIQAFKGEYAIFTYHSVISELIDLPHTIYEILEGCKLASQRGQNFVCIYPNNDLGSETILNALETLKNIPNFVCFPSMKFEYFLTLLKNAQFIIGNSSAGIREAGVYGVPCLNLGSRQYKRAQASHIMQVRENRHAILEAILEVKNKPRLEPAYHFGTGASVENFLNALTPALFDTPTQKYFAHD

SEQ ID NO: 5 - protein, 426 aa, Neisseria weaveri
MSNKSYAVDCFNAYLVYNFDQWLLSCKNGDSGISPFSFVGEFVYVGILMPKKKILCITGTRADFGKLKPLLAFLEQHSDFEMHLVITGMHMLKTYGYTYHEVIQEKYKHYYLLSNQTQNDSMSMVLGNTVSLISRLVEELRPDMIIIHGDRVEALAGASVGALNNCLVCHIEGGEVSGTIDDSIRHAVTKLSHLHLVANEQAVGRLVQLGEKKENIHIIGSPDLDVMNEDKLPSLAAAKQHYGIEFEQYGISIFHPVTTEVERTAYHAQMYFEALQESGKNIIAIYPNNDLGTEYIQAELGKIKSPKWKIFPSLRFEFFLVLLKHAEFIIGNSSAGIREAPLYGIPSIDVGTRQRNRHSGSSIIHSGYEKAQILNAVRQAGHCRRSEPDHTFQGGNGKSSVERFKEVMLLPELWSTPAQKQFIDRF

SEQ ID NO: 6 - protein, 372 aa, Halomonas meridiana
MKKIVFLTGTRADFGKLKSLMRNIEDDPNFELHVITTGMHMMKTYGSTYREVEREKYKHNYFVANQHFGEPMCSVLGNTISLVSRITHEIDPDLIVVHGDRLEALAGATVGALQSRLVCHVEGGERSGTVDELIRHSVSKLAHIHMVANEEAKKRLMQMGEDEGSIYIIGSPDLDVMNSDKLPSLEEVKKKYGIDFDNYAVSMFHPVTTEESKMGEYAEAYYNALQNSGDNFVSIYPNNDSGSTEIIEKLKKVSGGKIKSYPSIRFEYFLVLLKNAEYVIGNSSAGIREAPFYGVPTVNVGTRQSDRYMGSSIVNANYTAESILNSIQVAKDKRDIPPSKWFGDGDSTQKFAVAIRSTEFWERPVQKRFVDL

SEQ ID NO: 7 - protein, 380 aa, unidentified
  source 1..380: note = Parcubacteria group bacterium GW2011_GWA2_38_13
MNKKKKILIITGSRAEYGLLKPIIKGLMKNKKIEPHLLVTGMHTLKKFGFSVNEIIQDKMPIKAVVKILEKNDMLISLTKEILGIRKCCSSLKPDLILVLGDRDEAFAGAIVGGHMRIPVAHIHGGDVTGFVVDEYIRHSITKFSHLHFAASKKSFDRIIRLGEERWRVFVCGAPGIDSIRQGNFASKNSIAKKFGFDKNKKWLLIIHHPAPLDQIIFLRQIRPLLECVKNIDAEKIIIYPNSDTGSNIFIKEIHTCKKNNNFHIYKNLPKNDFLNIMNSADALLGNSSAGIIESGYFNLPTVNIGNRQKNRERGRNVIDSDYKKNTIKKAIARALSPQFKKNCKNMKSPYGNGHASEKIIKILEKHIDNKKLFLKKLTF

SEQ ID NO: 8 - protein, 377 aa, Flavobacterium sp.
  source 1..377: note = strain CF136
MSNSQKKILFLTGTRADFGKIKSLIQTLEIQQDFEVFVFVTGMHLQELYGYTLIEIERCNFKNVFTFENHTHETTMDLTLAKTIEGLSGYCKTINPDMIVVHGDRVETLAGAIVGSLNNILVAHIEGGEVSGTVDELIRHSVSKLSHVHFVSNVEAKKRLLQMGEIHESIFMIGSPDIDIMFSDQLPDLKKVKEYYNIDFEQYAIVMFHPVTTEIKEMQQYAISFVNSLLESNHNYVVIYPNNDLGSQFIIEAYEKLKMNSRFRIFPSLRFEYFLTLLKNSQFIIGNSSAGIREAPYYGIPIINIGTRQQNRAIHADIINTDYSERGIREALDLIVSHKVNASNEDFGQGNSNELFLECLQKQDIWQLNHQKQFRDF

SEQ ID NO: 9 - protein, 609 aa, Escherichia coli
  source 1..609: note = K-12 MG1655
MCGIVGAIAQRDVAEILLEGLRRLEYRGYDSAGLAVVDTEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYCHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLSRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 10 - protein, 268 aa, Pasteurella multocida
MDKFAEHEIPKAVIVAGNGESLSQIDYRLLPKNYDVFRCNQFYFEERYFLGNKIKAVFFTPGVFLEQYYTLYHLKRNNEYFVDNVILSSFNHPTVDLEKSQKIQALFIDVINGYEKYLSKLTAFDVYLRYKELYENQRITSGVYMCAVAIAMGYTDIYLTGIDFYQASEENYAFDNKKPNIIRLLPDFRKEKTLFSYHSKDIDLEALSFLQQHYHVNFYSISPMSPLSKHFPIPTVEDDCETTFVAPLKENYINDILLPPHFVYEKLG

SEQ ID NO: 11 - protein, 391 aa, Photobacterium damselae
MTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 12 - protein, 12 aa, synthetic construct
  VARIANT 1: note = X can be A, C, I, L or M
  VARIANT 2: note = X can be G, S or T
  VARIANT 4..5: note = S can be T
  VARIANT 6..9: note = X can be any amino acid
  VARIANT 10: note = D can be E
  VARIANT 11: note = X can be A, C, G, I, L, M, S or T
  VARIANT 12: note = X can be A, C, D, E, G, I, M, P, Q, S, T or V
XXNSSXXXXDXX

(19) United States
(12) Patent Application Publication
(10) Pub. No.: US 2026/0286409 A1
(43) Pub. Date: Sep. 24, 2026

(54) PRODUCTION OF A NEGATIVELY CHARGED OLIGOSACCHARIDE BY A CELL

(71) Applicant: INBIOSE N.V., Zwijnaarde (BE)

(72) Inventors: Joeri Beauprez, Zwijnaarde (BE); Thomas Decoene, Zwijnaarde (BE); Annelies Vercauteren, Zwijnaarde (BE); Tom Verhaeghe, Zwijnaarde (BE)

(21) Appl. No.: 19/473,464

(22) PCT Filed: Apr. 6, 2024
(86) PCT No.: PCT/EP2024/061529

§ 371 Date: Oct. 7, 2025

(30) Foreign Application Priority Data
Apr. 26, 2023 (EP) 23169954.7
Apr. 26, 2023 (LU) LU504075

(51) Int. Cl. C12P 19/04; C07K 14/435; C12N 1/00; C12N 9/02; C12N 9/10; C12N 9/24; C12N 9/90; C12N 15/63; C12P 19/18

(52) U.S. Cl. CPC C12P 19/04; C07K 14/792; C12N 1/00; C12N 9/0051; C12N 9/1048; C12N 9/2402; C12N 9/90; C12N 15/63; C12P 19/18; C12Y 108/03002; C12Y 302/01183; C12Y 503/04001; C12N 2830/34; C12N 2830/36

(57) ABSTRACT

Synthetic biology, metabolic engineering and cell cultivation. This disclosure provides a cell for production of a negatively charged, preferably sialylated, oligosaccharide wherein the cell is genetically engineered to possess or express, preferably to over-express a hydrolyzing UDP-N-acetyl-D-glucosamine-2-epimerase. This disclosure further provides use of the cell in a cultivation or incubation. This disclosure also describes methods for the production of a negatively charged, preferably sialylated, oligosaccharide using the cell as well as the purification of the negatively charged, preferably sialylated, oligosaccharide.

### CROSS-REFERENCE TO RELATED APPLICATIONS

This application is a national phase entry under 35 U.S.C. § 371 of International Patent Application PCT/EP2024/061529, filed Apr. 26, 2024, designating the United States of America and published as International Patent Publication WO 2024/223815 A1 on Oct. 31, 2024, which claims the benefit under Article 8 of the Patent Cooperation Treaty of European Paten Convention Patent Application Serial No. 23169954.7, filed Apr. 26, 2023 and Luxembourg Patent Application Serial No. LU504075, filed Apr. 26, 2023.

### STATEMENT REGARDING ELECTRONIC FILING OF A SEQUENCE LISTING

Pursuant to 37 C.F.R. § 1.831 through 1.835, a Sequence Listing XML file entitled “080-PCT sequence listing_v2.xml” 19 kilobytes in size, generated Oct. 10, 2025, has been submitted via EFS-Web in lieu of a paper copy, which Sequence Listing is hereby incorporated by reference into the specification.

### TECHNICAL FIELD

This disclosure is in the technical field of synthetic biology, metabolic engineering and cell cultivation. This disclosure provides a cell for production of a negatively charged, preferably sialylated, oligosaccharide wherein the cell is genetically engineered to possess or express, preferably to over-express a hydrolyzing UDP-N-acetyl-D-glucosamine-2-epimerase. This disclosure further provides use of the cell in a cultivation or incubation. This disclosure also describes methods for the production of a negatively charged, preferably sialylated, oligosaccharide using the cell as well as the purification of the negatively charged, preferably sialylated, oligosaccharide.

### BACKGROUND

Oligosaccharides are very diverse in chemical structure and are composed of miscellaneous monosaccharides, such as e.g., glucose, galactose, N-acetylglucosamine, xylose, rhamnose, fucose, mannose, N-acetylneuraminic acid, N-acetylgalactosamine, galactosamine, glucosamine, glucuronic acid, galacturonic acid. Oligosaccharides are widely distributed in all living organisms and play important roles in a variety of physiological and pathological processes, such as cell metastasis, signal transduction, intercellular adhesion, inflammation, and immune response. Economical production of these oligosaccharides is of utmost importance to fully benefit of their biological advantages. An important group of oligosaccharides comprises mammalian milk oligosaccharides (MMOs) and human milk oligosaccharides (HMOs) found in mammalian and human milk, respectively. The importance of milk oligosaccharides for animal and human infant nutrition is directly linked to their biological activities including protection of the neonate from pathogens, supporting development of the infant's immune system and cognitive abilities. In addition, milk oligosaccharides serve as a substrate for beneficial bacteria like Bifidobacteria or Lactobacilli. Milk oligosaccharides are further known to act as decoys to reduce the risk of infections by bacterial and viral pathogens, which adhere to human cells by binding to these cells' surface glycoproteins. Additionally, various milk oligosaccharides possess an anti-inflammatory effect and act as immunomodulators (e.g., reducing the risk of developing food allergies). Many of these milk oligosaccharides are negatively charged oligosaccharides. Among negatively charged HMOs, sialylated HMOs (SHMOs) were observed to support several beneficial effects as described in the art. Among the sialylated oligosaccharides in human milk, 3′sialyllactose, 6′sialyllactose, sialyllacto-N-tetraose a, sialyl lacto-N-tetraose b, sialyllacto-N-tetraose c and disialyllacto-N-tetraose are the most prevalent members.

Negatively charged oligosaccharides are found to be a complex structure and their chemical or (chemo-)enzymatic syntheses has been proven challenging: there are extensive difficulties, e.g., control of stereochemistry, formation of specific linkages, availability of feedstocks, etc. Therefore, alternative production methods have been developed, amongst which efforts in metabolic engineering of microorganisms to produce negatively charged oligosaccharides have been made.

### BRIEF SUMMARY

It is an object of the disclosure to provide for tools and methods by way of which a negatively charged, preferably sialylated, oligosaccharide can be produced, preferably in an efficient, time and cost-effective way and which yields high amounts of the desired oligosaccharide.

According to the disclosure, this and other objects are achieved by providing methods and a cell for the production of a negatively charged, preferably sialylated, oligosaccharide. This disclosure also provides methods for the purification of the negatively charged, preferably sialylated, oligosaccharide. Furthermore, the disclosure provides a cell that is genetically engineered as described herein to possess or express, preferably to over-express, a hydrolyzing UDP-N-acetyl-D-glucosamine-2-epimerase wherein the hydrolyzing UDP-N-acetyl-D-glucosamine-2-epimerase comprises an amino acid sequence comprising a conserved motif with SEQ ID NO: 12 and having hydrolyzing UDP-N-acetyl-D-glucosamine-2-epimerase activity. This disclosure also provides a purified negatively charged, preferably sialylated, oligosaccharide by the above-referenced process. Further benefits of the teachings of this disclosure will be apparent to one skilled in the art from reading this disclosure.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various aspects and embodiments of the disclosure disclosed herein are to be understood not only in the order and context specifically described in this specification, but to include any order and any combination thereof. Each embodiment as identified herein may be combined unless otherwise indicated. All publications, patents, and patent applications mentioned in this specification are herein incorporated by reference to the same extent as if each individual publication, patent, or patent application was specifically and individually indicated to be incorporated by reference in its entirety. Whenever the context requires, unless specifically stated otherwise, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the specification, there have been disclosed embodiments of the disclosure, and although specific terms are employed, the terms are used in a descriptive sense only and not for purposes of limitation, the scope of the disclosure being set forth in the following claims. It must be understood that the illustrated embodiments have been set forth only for the purposes of example and that it should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps, unless specifically stated otherwise.

Throughout the disclosure, unless explicitly stated otherwise, the features “synthesize”, “synthesized” and “synthesis” are interchangeably used with the features “produce”, “produced” and “production”, respectively. Throughout the disclosure, unless explicitly stated otherwise, the expressions “capable of . . . <verb>” and “capable to . . . <verb>” are preferably replaced with the active voice of the verb and vice versa. For example, the expression “capable of expressing” is preferably replaced with “expresses” and vice versa, i.e., “expresses” is preferably replaced with “capable of expressing”. In this document and in its claims, the verb “to comprise”, “to have” and “to contain” and their conjugations are used in their non-limiting sense to mean that items following the word are included, but items not specifically mentioned are not excluded. Throughout the disclosure, the verb “to comprise” may be replaced by “to consist” or “to consist essentially of” and vice versa. In addition, the verb “to consist” may be replaced by “to consist essentially of” meaning that a composition as defined herein may comprise additional component(s) than the ones specifically identified, the additional component(s) not altering the unique characteristic of the disclosure. In this document and in its claims, unless specifically stated otherwise, the verbs “to comprise”, “to have” and “to contain”, and their conjugations, may be replaced by “to consist of” (and its conjugations) or “to consist essentially of” (and its conjugations) and vice versa. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

Throughout the document and in the claims, unless explicitly stated otherwise, the articles “a” and “an” are preferably replaced by “at least one”, more preferably “at least two”, even more preferably by “at least three”, even more preferably by “at least four”, even more preferably by “at least five”, even more preferably by “at least six”, most preferably by “at least two”. The word “about” or “approximately” when used in association with a numerical value (e.g., “about 10”) or with a range (e.g., “about x to approximately y”) preferably means that the value or range is interpreted as being as accurate as the method used to measure it. If no error margins are specified, the expression “about” or “approximately” when used in association with a numerical value is interpreted as having the same round-off as the given value. Throughout this document and its claims, unless otherwise stated, the expression “from x to y”, wherein x and y represent numerical values, refers to a range of numerical values wherein x is the lower value of the range and y is the upper value of the range. Herein, x and y are also included in the range.

According to the disclosure, the term “polynucleotide(s)” generally refers to any polyribonucleotide or polydeoxyribonucleotide, which may be unmodified RNA or DNA or modified RNA or DNA. DNAs or RNAs comprising unusual bases, such as inosine, or modified bases, such as tritylated bases, are to be understood to be covered by the term “polynucleotides”. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques as well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Polypeptides may be branched or cyclic, with or without branching. Cyclic, branched and branched circular polypeptides may result from post-translational natural processes and may be made by entirely synthetic methods, as well.

The term “polynucleotide encoding a polypeptide” as used herein encompasses polynucleotides that include a sequence encoding a polypeptide of the disclosure. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Similarly, a “synthetic” sequence, as the term is used herein, means any sequence that has been generated synthetically and not directly isolated from a natural source. “Synthesized”, as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic makeup and is replicated.

The terms “recombinant” or “transgenic” or “metabolically engineered” or “genetically engineered” as used herein with reference to a cell or host cell are used interchangeably and indicates that the cell replicates a heterologous nucleic acid, or expresses a peptide or protein encoded by a heterologous nucleic acid (i.e., a sequence “foreign to the cell” or a sequence “foreign to the location or environment in the cell”). Such cells are described to be transformed with at least one heterologous or exogenous gene or are described to be transformed by the introduction of at least one heterologous or exogenous gene. Recombinant or metabolically engineered or genetically engineered or transgenic cells can contain genes that are not found within the native (non-recombinant) form of the cell. Recombinant cells can also contain genes found in the native form of the cell wherein the genes are modified and re-introduced into the cell by artificial means. The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; CrispR; riboswitch; recombineering; ssDNA mutagenesis; transposon mutagenesis and related techniques as known to a person skilled in the art. Accordingly, a “recombinant polypeptide” is one that has been produced by a recombinant cell. The terms also encompass cells that have been modified by removing a nucleic acid endogenous to the cell by way of common well-known technologies for a skilled person (like e.g., knocking-out genes).

A “heterologous sequence” or a “heterologous nucleic acid”, as used herein, is one that originates from a source foreign to the particular cell (e.g., from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g., by transfection, transformation, conjugation or transduction, into the genome of the host cell, wherein techniques may be applied that will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” or “engineered” cell as used within the context of the disclosure refers to a cell that is genetically engineered.

The term “endogenous” within the context of the present disclosure refers to any polynucleotide, polypeptide or protein sequence that is a natural part of a cell and is occurring at its natural location in the cell chromosome and of which the control of expression has not been altered compared to the natural control mechanism acting on its expression. The term “exogenous” refers to any polynucleotide, polypeptide or protein sequence that originates from outside the cell under study and not a natural part of the cell or which is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism. In contrast a “homologous” polynucleotide, gene, nucleic acid, polypeptide, or enzyme is used herein to denote a polynucleotide, gene, nucleic acid, polypeptide, or enzyme that is derived from the host organism. When referring to a gene regulatory sequence or to an auxiliary nucleic acid sequence used for maintaining or manipulating a gene sequence (e.g., a promoter, a 5′ untranslated region, 3′untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “modified expression” of a gene relates to a change in expression compared to the wild-type expression of the gene in any phase of the production process of the desired negatively charged, preferably sialylated, oligosaccharide. The modified expression is either a lower or higher expression compared to the wild-type, wherein the term “higher expression” is also defined as “overexpression” of the gene in the case of an endogenous gene or “expression” in the case of a heterologous gene that is not present in the wild-type strain. Lower expression is obtained by way of common well-known technologies for a skilled person (such as the usage of siRNA, CrispR, CrispRi, riboswitch, recombineering, homologous recombination, ssDNA mutagenesis, RNAi, miRNA, asRNA, mutating genes, knocking-out genes, transposon mutagenesis, etc.) which are used to change the genes in such a way that they are “less-able” (i.e., statistically significantly ‘less-able’ compared to a functional wild-type gene) or completely unable (such as knocked-out genes) to produce functional final products. The term “riboswitch” as used herein is defined to be part of the messenger RNA that folds into intricate structures that block expression by interfering with translation. Binding of an effector molecule induces conformational change(s) permitting regulated expression post-transcriptionally. Next to changing the gene of interest in such a way that lower expression is obtained as described above, lower expression can also be obtained by changing the transcription unit, the promoter, an untranslated region, the ribosome binding site, the Shine Dalgamo sequence or the transcription terminator. Lower expression or reduced expression can, for instance, be obtained by mutating one or more base pairs in the promoter sequence or changing the promoter sequence fully to a constitutive promoter with a lower expression strength compared to the wild-type or an inducible promoter, which result in regulated expression or a repressible promoter, which results in regulated expression. Overexpression or expression is obtained by way of common well-known technologies for a skilled person (such as the usage of artificial transcription factors, de novo design of a promoter sequence, ribosome engineering, introduction or re-introduction of an expression module at euchromatin, usage of high-copy-number plasmids), wherein the gene is part of an “expression cassette” that relates to any sequence in which a promoter sequence, untranslated region sequence (containing either a ribosome binding sequence, Shine Dalgarno or Kozak sequence), a coding sequence and optionally a transcription terminator is present, and leading to the expression of a functional active protein. The expression is either constitutive or conditional or regulated or tuneable.

The term “constitutive expression” is defined as expression that is not regulated by transcription factors other than the subunits of RNA polymerase (e.g., the bacterial sigma factors like s⁷⁰, s⁵⁴, or related s-factors and the yeast mitochondrial RNA polymerase specificity factor MTF1 that co-associate with the RNA polymerase core enzyme) under certain growth conditions. Non-limiting examples of such transcription factors are CRP, LacI, ArcA, Cra, IclR in E. coli, or, Aft2p, Crz1p, Skn7 in Saccharomyces cerevisiae, or, DeoR, GntR, Fur in B. subtills. These transcription factors bind on a specific sequence and may block or enhance expression in certain growth conditions. The RNA polymerase is the catalytic machinery for the synthesis of RNA from a DNA template. RNA polymerase binds a specific DNA sequence to initiate transcription, for instance, via a sigma factor in prokaryotic hosts or via MTF1 in yeasts. Constitutive expression offers a constant level of expression with no need for induction or repression.

The term “regulated expression” is defined as expression that is regulated by transcription factors other than the subunits of RNA polymerase (e.g., bacterial sigma factors) under certain growth conditions. Examples of such transcription factors are described above. Commonly expression regulation is obtained by way of an inducer, such as but not limited to IPTG, arabinose, rhamnose, fucose, allo-lactose or pH shifts, or temperature shifts or carbon depletion or substrates or the produced product.

The term “control sequences” refers to sequences recognized by the cells transcriptional and translational systems, allowing transcription and translation of a polynucleotide sequence to a polypeptide. Such DNA sequences are thus necessary for the expression of an operably linked coding sequence in a particular host cell, cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. The control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of the polynucleotide to a polypeptide.

Generally, “operably linked” means that the DNA sequences being linked are contiguous, and, in the case of a secretory leader, contiguous and in reading phase. However, enhancers do not have to be contiguous.

The term “wildtype” refers to the commonly known genetic or phenotypical situation as it occurs in nature.

The term “modified expression of a protein” as used herein refers to i) higher expression or overexpression of an endogenous protein, ii) expression of a heterologous protein, iii) expression and/or overexpression of a variant protein that has a higher activity compared to the wild-type (i.e., native in the expression host) protein, iv) reduced expression of an endogenous protein or v) expression and/or overexpression of a variant protein that has a reduced activity compared to the wild-type (i.e., native in the expression host) protein. Preferably, the term “modified expression of a protein” as used herein refers to i) higher expression or overexpression of an endogenous protein, ii) expression of a heterologous protein or iii) expression and/or overexpression of a variant protein that has a higher activity compared to the wild-type (i.e., native in the expression host) protein.

The term “modified activity” of a protein relates to a non-native activity of the protein in any phase of the production process of the desired negatively charged, preferably sialylated, oligosaccharide. The term “non-native”, as used herein with reference to the activity of a protein indicates that the protein has been modified to have an abolished, impaired, reduced, delayed, higher, accelerated or improved activity compared to the native activity of the protein. A modified activity of a protein is obtained by modified expression of the protein or is obtained by expression of a modified, i.e., mutant form of the protein. A mutant form of the protein can be obtained by expression of a mutant form of the gene encoding the protein, e.g., comprising a deletion, an insertion and/or a mutation of one or more nucleotides compared to the native gene sequence. A mutant form of a gene can be obtained by techniques well-known to a person skilled in the art, such as but not limited to site-specific mutation; CrispR; riboswitch; recombineering; ssDNA mutagenesis; transposon mutagenesis.

The term “non-native”, as used herein with reference to a cell producing a negatively charged, preferably sialylated, oligosaccharide, indicates that the negatively charged, preferably sialylated, oligosaccharide is i) not naturally produced or ii) when naturally produced not in the same amounts by the cell; and that the cell has been genetically engineered to be able to produce the negatively charged, preferably sialylated, oligosaccharide or to have a higher production of the negatively charged, preferably sialylated, oligosaccharide.

“Variant(s)” as the term is used herein, is a polynucleotide or polypeptide that differs from a reference polynucleotide or polypeptide, respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

In some embodiments, the disclosure contemplates making functional variants by modifying the structure of an enzyme as used in the disclosure. Variants can be produced by amino acid substitution, deletion, addition, or combinations thereof. For instance, a variant can be produced as a fusion protein comprising at least one portion of an enzyme of the disclosure fused to at least one portion comprising a peptide tag. The peptide tag may be used to assist protein folding of the enzyme, assist post expression purification, protect the enzyme from the action of degradative enzymes, and/or assist the enzyme in passing through the cell membrane. Examples of the peptide tag comprise, e.g., a SUMO tag, an MBP tag, a His tag, a FLAG tag, a Strep-II tag, a Halo-tag, a NusA tag, thioredoxin, GST and/or a Fh8-tag. The fusion protein may be designed to include at least one cleavable peptide linker so that the enzyme of interest can be subsequently recovered from the fusion protein. The fusion protein may be designed to include a plurality of inclusion body tags, cleavable peptide linkers, and regions encoding the enzyme of interest. In case of amino acid substitution, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide.

“Fragment”, with respect to a polynucleotide, refers to a clone or any part of a polynucleotide molecule, particularly a part of a polynucleotide that retains a usable, functional characteristic of the full-length polynucleotide molecule. Useful fragments include oligonucleotides and polynucleotides that may be used in hybridization or amplification technologies or in the regulation of replication, transcription or translation. A “polynucleotide fragment” refers to any subsequence of a polynucleotide SEQ ID NO, typically, comprising or consisting of at least about 9, 10, 11, 12 consecutive nucleotides from the polynucleotide SEQ ID NO, for example, at least about 30 nucleotides or at least about 50 nucleotides of any of the polynucleotide sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide. As such, a fragment of a polynucleotide SEQ ID NO preferably means a nucleotide sequence that comprises or consists of the polynucleotide SEQ ID NO wherein no more than about 200, 150, 100, 50 or 25 consecutive nucleotides are missing, preferably no more than about 50 consecutive nucleotides are missing, and which retains a usable, functional characteristic (e.g., activity) of the full-length polynucleotide molecule, which can be assessed by the skilled person through routine experimentation. Alternatively, a fragment of a polynucleotide SEQ ID NO preferably means a nucleotide sequence that comprises or consists of an amount of consecutive nucleotides from the polynucleotide SEQ ID NO and wherein the amount of consecutive nucleotides is at least 50%, 60%, 70%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 100%, preferably at least 80%, more preferably at least 85%, even more preferably at least 87%, even more preferably at least 90%, even more preferably at least 95%, most preferably at least 97%, of the full-length of the polynucleotide SEQ ID NO and retains a usable, functional characteristic (e.g., activity) of the full-length polynucleotide molecule that can be routinely assessed by the skilled person. As such, a fragment of a polynucleotide SEQ ID NO preferably means a nucleotide sequence that comprises or consists of the polynucleotide SEQ ID NO, wherein an amount of consecutive nucleotides is missing and wherein the amount is no more than 50%, 40%, 30% of the full-length of the polynucleotide SEQ ID NO, preferably no more than 20%, 15%, 10%, 9%, 8%, 7%, 6%, 5%, 4.5%, 4%, 3.5%, 3%, 2.5%, 2%, 1.5%, 1%, 0.5%, more preferably no more than 15%, even more preferably no more than 10%, even more preferably no more than 5%, most preferably no more than 2.5%, of the full-length of the polynucleotide SEQ ID NO and wherein the fragment retains a usable, functional characteristic (e.g., activity) of the full-length polynucleotide molecule that can be routinely assessed by the skilled person.

“Fragment”, with respect to a polypeptide, refers to a subsequence of the polypeptide that performs at least one biological function of the intact polypeptide in substantially the same manner, or to a similar extent, as does the intact polypeptide. A “subsequence of the polypeptide” or “a stretch of amino acid residues” as described herein refers to a sequence of contiguous amino acid residues derived from the polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example, at least about 10 amino acid residues in length, for example, at least about 20 amino acid residues in length, for example, at least about 30 amino acid residues in length, for example, at least about 100 amino acid residues in length, for example, at least about 150 amino acid residues in length, for example, at least about 200 amino acid residues in length. As such, a fragment of a polypeptide SEQ ID NO (or UniProt ID) preferably means a polypeptide sequence that comprises or consists of the polypeptide SEQ ID NO (or UniProt ID) wherein no more than about 200, 150, 125, 100, 80, 60, 50, 40, 30, 20 or 15 consecutive amino acid residues are missing, preferably no more than about 100 consecutive amino acid residues are missing, more preferably no more than about 50 consecutive amino acid residues are missing, even more preferably no more than about 40 consecutive amino acid residues are missing, and performs at least one biological function of the intact polypeptide in substantially the same manner, preferably to a similar or greater extent, as does the intact polypeptide, which can be routinely assessed by the skilled person. Alternatively, a fragment of a polypeptide SEQ ID NO (or UniProt ID) preferably means a polypeptide sequence that comprises or consists of an amount of consecutive amino acid residues from the polypeptide SEQ ID NO (or UniProt ID) and wherein the amount of consecutive amino acid residues is at least 50%, 60%, 70%, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 100%, preferably at least 80%, more preferably at least 85%, even more preferably at least 87%, even more preferably at least 90%, even more preferably at least 95%, most preferably at least 97% of the full-length of the polypeptide SEQ ID NO (or UniProt ID) and which performs at least one biological function of the intact polypeptide in substantially the same manner, preferably to a similar or greater extent, as does the intact polypeptide, which can be routinely assessed by the skilled person. As such, a fragment of a polypeptide SEQ ID NO (or UniProt ID) preferably means a polypeptide sequence that comprises or consists of the polypeptide SEQ ID NO (or UniProt ID), wherein an amount of consecutive amino acid residues is missing and wherein the amount is no more than 50%, 40%, 30% of the full-length of the polypeptide SEQ ID NO (or UniProt ID), preferably no more than 20%, 15%, 10%, 9%, 8%, 7%, 6%, 5%, 4.5% 4%, 3.5% 3%, 2.5%, 2%, 1.5%, 1%, 0.5%, more preferably no more than 15%, even more preferably no more than 10%, even more preferably no more than 5%, most preferably no more than 2.5%, of the full-length of the polypeptide SEQ ID NO (or UniProt ID) and which performs at least one biological function of the intact polypeptide in substantially the same manner, preferably to a similar or greater extent, as does the intact polypeptide, which can be routinely assessed by the skilled person.

Throughout the disclosure, the sequence of a polypeptide can be represented by a SEQ ID NO or alternatively by an UniProt ID. Therefore, the terms “polypeptide SEQ ID NO” and “polypeptide UniProt ID” can be interchangeably used, unless explicitly stated otherwise.

A “functional fragment” of a polypeptide has at least one property or activity of the polypeptide from which it is derived, preferably to a similar or greater extent. A functional fragment can, for example, include a functional domain or conserved domain of a polypeptide. It is understood that a polypeptide or a fragment thereof may have conservative amino acid substitutions, which have substantially no effect on the polypeptide's activity. By conservative substitutions is intended substitutions of one hydrophobic amino acid for another or substitution of one polar amino acid for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa.

Homologous sequences as used herein describes those nucleotide sequences that have sequence similarity and encode polypeptides that share at least one functional characteristic such as a biochemical activity. More specifically, the term “functional homolog” as used herein describes those polypeptides that have sequence similarity (in other words, homology) and at the same time have at least one functional similarity such as a biochemical activity (Altenhoff et al., PLoS Comput. Biol. 8 (2012) e1002514).

Homologs can be identified by analysis of nucleotide and polypeptide sequence alignments. For example, performing a query on a database of nucleotide or polypeptide sequences can identify homologs of the nucleotides or polypeptides of interest. Sequence analysis can involve BLAST, Reciprocal BLAST, or PSI-BLAST analysis of non-redundant databases using the amino acid sequence of a reference polypeptide sequence. The amino acid sequence is, in some instances, deduced from the nucleotide sequence. Typically, those polypeptides in the database that have greater than 40% sequence identity to a polypeptide of interest are candidates for further evaluation for suitability as a homologous polypeptide. Amino acid sequence similarity allows for conservative amino acid substitutions, such as substitution of one hydrophobic residue for another or substitution of one polar residue for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa. If desired, manual inspection of such candidates can be carried out in order to narrow the number of candidates to be further evaluated.

A domain can be characterized, for example, by a Pfam (El-Gebali et al., Nucleic Acids Res. 47 (2019) D427-D432), an IPR (InterPro domain) (http://ebi.ac.uk/interpro) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (https://www.ncbi.nlm.nih.gov/cdd) (Lu et al., Nucleic Acids Res. 48 (2020) D265-D268), a PTHR domain (http://www.pantherdb.org) (Mi et al., Nucleic Acids. Res. 41 (2013) D377-D386; Thomas et al., Genome Research 13 (2003) 2129-2141) or a PATRIC identifier or PATRIC DB global family domain (https://www.patricbrc.org/) (Davis et al., Nucleic Acids Res. 48(D1) (2020) D606-D612). Protein or polypeptide sequence information and functional information can be provided by a comprehensive resource for protein sequence and annotation data like e.g., the Universal Protein Resource (UniProt) (www.uniprot.org) (Nucleic Acids Res. 2021, 49(D1), D480-D489). UniProt comprises the expertly and richly curated protein database called the UniProt Knowledgebase (UniProtKB), together with the UniProt Reference Clusters (UniRef) and the UniProt Archive (UniParc). The UniProt identifiers (UniProt ID) are unique for each protein present in the database. Throughout the disclosure, the sequence of a polypeptide is represented by a SEQ ID NO or an UniProt ID. Unless stated otherwise, the UniProt IDs of the proteins described correspond to their sequence version 01 as present in the UniProt Database (www.uniprot.org) version release 2021_03 and consulted on 9 Jun. 2021.

InterPro provides functional analysis of proteins by classifying them into families and predicting domains and important sites. To classify proteins in this way, InterPro uses predictive models, known as signatures, provided by several different databases (referred to as member databases) that make up the InterPro consortium. Protein signatures from these member databases are combined into a single searchable resource, capitalizing on their individual strengths to produce a powerful integrated database and diagnostic tool.

It should be understood for those skilled in the art that for the databases used herein, comprising Pfam 32.0 (released September 2018), CDD v3.17 (released 3 Apr. 2019), eggnogdb 4.5.1 (released September 2016), InterPro 90.0 (released 4 Aug. 2022), PANTHER 17.0 (released 23 Feb. 2022) and PATRIC 3.6.9 (released March 2020), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

The terms “identical” or “percent identity” or “% identity” in the context of two or more nucleic acid or polypeptide sequences, refer to two or more sequences or subsequences that are the same or have a specified percentage of nucleotides or amino acid residues that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the % sequence identity for the test sequence(s) relative to the reference sequence, based on the designated program parameters. The percentage of sequence identity can be, preferably is, determined by alignment of the two sequences and identification of the number of positions with identical residues divided by the number of residues in the shorter of the sequences×100. Percent identity may be calculated globally over the full-length sequence of a given SEQ ID NO, i.e., the reference sequence, resulting in a global % identity score. Alternatively, % identity may be calculated over a partial sequence of the reference sequence, resulting in a local percent identity score. A partial sequence preferably means at least about 50%, 60%, 70%, 80%, 90% or 95% of the full-length reference sequence. In another preferred embodiment, a partial sequence of a reference polypeptide sequence means a stretch of at least 150 amino acid residues up to the total number of amino acid residues of a reference polypeptide sequence. In another more preferred embodiment, a partial sequence of a reference polypeptide sequence means a stretch of at least 200 amino acid residues up to the total number of amino acid residues of a reference polypeptide sequence. Using the full-length of the reference sequence in a local sequence alignment results in a global percent identity score between the test and the reference sequence.

Percent identity can be determined using different algorithms like, for example, BLAST and PSI-BLAST (Altschul et al., 1990, J Mol Biol 215:3, 403-410; Altschul et al., 1997, Nucleic Acids Res 25: 17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle.

As used herein, a polypeptide comprising or consisting of an amino acid sequence having 80% or more sequence identity over a stretch of at least 150 amino acid residues of a reference polypeptide sequence is to be understood as that the amino acid sequence 80%, 81%, 82%, 83%, 84%, 85%, 86%, 87%, 88%, 89%, 90%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% sequence identity over a stretch of at least 150 amino acid residues of the reference polypeptide sequence.

As used herein, a polypeptide comprising or consisting of an amino acid sequence having 80% or more sequence identity over a stretch of at least 200 amino acid residues of a reference polypeptide sequence is to be understood as that the amino acid sequence 80%, 81%, 82%, 83%, 84%, 85%, 86%, 87%, 88%, 89%, 90%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% sequence identity over a stretch of at least 200 amino acid residues of the reference polypeptide sequence.

As used herein, a polypeptide comprising or consisting of an amino acid sequence having 80% or more sequence identity to the full-length sequence of a reference polypeptide sequence is to be understood as that the amino acid sequence has 80%, 81%, 82%, 83%, 84%, 85%, 86%, 87%, 88%, 89%, 90%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5%, 99.6%, 99.7%, 99.8%, 99.9%, 100% sequence identity to the full-length of the amino acid sequence of the reference polypeptide sequence.

Throughout the disclosure, unless explicitly specified otherwise, a polypeptide comprising, consisting of or having an amino acid sequence having 80% or more sequence identity to the full-length amino acid sequence of a reference polypeptide, usually indicated with a SEQ ID NO or UniProt ID, preferably has 80%, 85%, 90%, 91%, 92%, 93%, 94%, 95%, 96%, 97%, 98% or 99%, more preferably has at least 85%, even more preferably has at least 90%, sequence identity to the full length reference sequence. Additionally, unless explicitly specified otherwise, a polynucleotide sequence comprising, consisting of or having a nucleotide sequence having 80%, 85%, 90%, 91%, 92%, 93%, 94%, 95%, 96%, 97%, 98% or 99%, more preferably has at least 85%, even more preferably has at least 90% sequence identity to the full-length reference sequence.

For the purposes of this disclosure, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50. In a preferred embodiment, sequence identity is calculated based on the full-length sequence of a given SEQ ID NO, i.e., the reference sequence, or a part thereof. Part thereof preferably means at least 50%, 60%, 70%, 80%, 90% or 95% of the complete reference sequence.

The terms “sialic acid”, “N-acetylneuraminate”, “N-acylneuraminate”, “N-acetylneuraminic acid” are used interchangeably and refer to an acidic sugar comprising but not limited to Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4; Neu4,5,7,8,9Ac5; Neu5Gc and 2-keto-3-deoxymanno-octulonic acid (KDO).

Neu4Ac is also known as 4-O-acetyl-5-amino-3,5-dideoxy-D-glycero-D-galacto-non-2-ulopyranosonic acid or 4-O-acetyl neuraminic acid and has C11H19NO9 as molecular formula. Neu5Ac is also known as 5-acetamido-3,5-dideoxy-D-glycero-D-galacto-non-2-ulopyranosonic acid, D-glycero-5-acetamido-3,5-dideoxy-D-galacto-non-2-ulo-pyranosonic acid, 5-(acetylamino)-3,5-dideoxy-D-glycero-D-galacto-2-nonulopyranosonic acid, 5-(acetylamino)-3,5-dideoxy-D-glycero-D-galacto-2-nonulosonic acid, 5-(acetylamino)-3,5-dideoxy-D-glycero-D-galacto-non-2-nonulosonic acid or 5-(acetylamino)-3,5-dideoxy-D-glycero-D-galacto-non-2-ulopyranosonic acid and has C11H19NO9 as molecular formula. Neu4,5Ac2 is also known as N-acetyl-4-O-acetylneuraminic acid, 4-O-acetyl-N-acetylneuraminic acid, 4-O-acetyl-N-acetylneuraminate, 4-acetate 5-acetamido-3,5-dideoxy-D-glycero-D-galacto-nonulosonate, 4-acetate 5-(acetylamino)-3,5-dideoxy-D-glycero-D-galacto-2-nonulosonate, 4-acetate 5-acetamido-3,5-dideoxy-D-glycero-D-galacto-nonulosonic acid or 4-acetate 5-(acetylamino)-3,5-dideoxy-D-glycero-D-galacto-2-nonulosonic acid and has C13H21NO10 as molecular formula. Neu5,7Ac2 is also known as 7-O-acetyl-N-acetylneuraminic acid, N-acetyl-7-O-acetylneuraminic acid, 7-O-acetyl-N-acetylneuraminate, 7-acetate 5-acetamido-3,5-dideoxy-D-glycero-D-galacto-nonulosonate, 7-acetate 5-(acetylamino)-3,5-dideoxy-D-glycero-D-galacto-2-nonulosonate, 7-acetate 5-acetamido-3,5-dideoxy-D-glycero-D-galacto-nonulosonic acid or 7-acetate 5-(acetylamino)-3,5-dideoxy-D-glycero-D-galacto-2-nonulosonic acid and has C13H21NO10 as molecular formula. Neu5,8Ac2 is also known as 5-n-acetyl-8-o-acetyl neuraminic acid and has C13H21NO10 as molecular formula. Neu5,9Ac2 is also known as N-acetyl-9-O-acetylneuraminic acid, 9-anana, 9-O-acetylsialic acid, 9-O-acetyl-N-acetylneuraminic acid, 5-n-acetyl-9-O-acetyl neuraminic acid, N,9-O-diacetylneuraminate or N,9-O-diacetylneuraminate and has C13H21NO10 as molecular formula. Neu4,5,9Ac3 is also known as 5-N-acetyl-4,9-di-O-acetylneuraminic acid. Neu5,7,9Ac3 is also known as 5-N-acetyl-7,9-di-O-acetylneuraminic acid. Neu5,8,9Ac3 is also known as 5-N-acetyl-8,9-di-O-acetylneuraminic acid. Neu4,5,7,9Ac4 is also known as 5-N-acetyl-4,7,9-tri-O-acetylneuraminic acid. Neu5,7,8,9Ac4 is also known as 5-N-acetyl-7,8,9-tri-O-acetylneuraminic acid. Neu4,5,7,8,9Ac5 is also known as 5-N-acetyl-4,7,8,9-tetra-O-acetylneuraminic acid. Neu5Gc is also known as N-glycolyl-neuraminic acid, N-glycolylneuraminic acid, N-glycolylneuraminate, N-glycoloyl-neuraminate, N-glycoloyl-neuraminic acid, N-glycoloylneuraminic acid, 3,5-dideoxy-5-((hydroxyacetyl)amino)-D-glycero-D-galacto-2-nonulosonic acid, 3,5-dideoxy-5-(glycoloylamino)-D-glycero-D-galacto-2-nonulopyranosonic acid, 3,5-dideoxy-5-(glycoloylamino)-D-glycero-D-galacto-non-2-ulopyranosonic acid, 3,5-dideoxy-5-[(hydroxyacetyl)amino]-D-glycero-D-galacto-non-2-ulopyranosonic acid, D-glycero-5-glycolylamido-3,5-dideoxy-D-galacto-non-2-ulo-pyranosonic acid and has C11H19NO10 as molecular formula. 2-keto-3-deoxymanno-octulonic acid is also known as KDO, Kdo, kdo, 2-keto-3-deoxy-D-mannooctanoic acid, 2-oxo-3-deoxy-D-mannooctonic acid, 3-deoxy-D-manno-2-octulosonic acid, 3-deoxy-D-manno-oct-2-ulo-pyranosonic acid, 3-deoxy-D-manno-oct-2-ulosonic acid, 3-deoxy-D-manno-octulosonic acid, 3-deoxy-D-manno-oct-2-ulopyranosonic acid, ketodeoxyoctonic acid, ketodeoxyoctulonic acid, (6R)-6-(hydroxymethyl)-1-carboxy-2-deoxy-D-lyxo-hexopyranose, keto-deoxy-octulonic acid and has C8H1408 as molecular formula.

The term “glycosyltransferase” as used herein refers to an enzyme capable to catalyze the transfer of a sugar moiety of a donor to a specific acceptor, forming glycosidic bonds. The donor can be a precursor as defined herein. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org). As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

Sialyltransferases are glycosyltransferases that transfer a sialic acid (like Neu5Ac) from a donor (like CMP-Neu5Ac) onto an acceptor. Sialyltransferases comprise alpha-2,3-sialyltransferases, alpha-2,6-sialyltransferases and alpha-2,8-sialyltransferases that catalyze the transfer of a sialic acid onto an acceptor via alpha-glycosidic bonds. Sialyltransferases can be found but are not limited to the GT29, GT42, GT52, GT80, GT97 and GT100 CAZy families.

The terms “alpha-2,3-sialyltransferase”, “alpha 2,3 sialyltransferase”, “3-sialyltransferase, “α-2,3-sialyltransferase”, “a 2,3 sialyltransferase”, “3 sialyltransferase”, “3-ST” or “3ST” or “a23-ST” as used in the disclosure, are used interchangeably and refer to a glycosyltransferase that catalyzes the transfer of sialic acid from the donor CMP-sialic acid, to the acceptor molecule in an alpha-2,3-linkage.

The terms “alpha-2,6-sialyltransferase”, “alpha 2,6 sialyltransferase”, “6-sialyltransferase, “α-2,6-sialyltransferase”, “a 2,6 sialyltransferase”, “6 sialyltransferase”, “6-ST” or “6ST” or “a26-ST” as used in the disclosure, are used interchangeably and refer to a glycosyltransferase that catalyzes the transfer of sialic acid from the donor CMP-sialic acid, to the acceptor molecule in an alpha-2,6-linkage.

The terms “alpha-2,8-sialyltransferase”, “alpha 2,8 sialyltransferase”, “8-sialyltransferase, “α-2,8-sialyltransferase”, “a 2,8 sialyltransferase”, “8 sialyltransferase”, “8-ST” or “8ST” or “a28-ST” as used in the disclosure, are used interchangeably and refer to a glycosyltransferase that catalyzes the transfer of sialic acid from the donor CMP-sialic acid, to the acceptor molecule in an alpha-2,8-linkage.

The term “monosaccharide” as used herein refers to a sugar that is not decomposable into simpler sugars by hydrolysis, is classed as an aldose, a ketose, a deoxysugar, a deoxy-aminosugar, a uronic acid, an aldonic acid, a ketoaldonic acid, an aldaric acid or a sugar alcohol, and contains one or more hydroxyl groups per molecule. Monosaccharides are saccharides containing only one simple sugar.

The term “phosphorylated monosaccharide” as used herein refers to a monosaccharide that is phosphorylated. Examples of phosphorylated monosaccharides include but are not limited to glucose-1-phosphate, glucose-6-phosphate, glucose-1,6-bisphosphate, galactose-1-phosphate, fructose-6-phosphate, fructose-1,6-bisphosphate, fructose-1-phosphate, glucosamine-1-phosphate, glucosamine-6-phosphate, N-acetylglucosamine-1-phosphate, mannose-1-phosphate, mannose-6-phosphate or fucose-1-phosphate. Some, but not all, of these phosphorylated monosaccharides are precursors or intermediates for the production of activated monosaccharide.

The terms “activated monosaccharide”, “nucleotide-activated sugar”, “nucleotide-sugar”, “activated sugar”, “nucleoside” or “nucleotide donor” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid, GDP-fucose (GDP-Fuc), GDP-rhamnose and UDP-xylose. Nucleotide-sugars act as glycosyl donors in glycosylation reactions. Glycosylation reactions are reactions that are catalyzed by glycosyltransferases.

The term “CMP-sialic acid” as used herein refers to a nucleotide-activated form of sialic acid comprising but not limited to CMP-Neu5Ac, CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc) and CMP-KDO.

The term “disaccharide” as used herein refers to a saccharide polymer containing two simple sugars, i.e., monosaccharides. Examples of disaccharides comprise lactose (Gal-β1,4-Glc), lacto-N-biose (Gal-β1,3-GlcNAc), N-acetyllactosamine (Gal-β1,4-GlcNAc), LacDiNAc (GalNAc-β1,4-GlcNAc), N-acetylgalactosaminylglucose (GalNAc-β1,4-Glc), Neu5Ac-α2,3-Gal, Neu5Ac-α2,6-Gal, fucopyranosyl-(1-4)-N-glycolylneuraminic acid (Fuc-(1-4)-Neu5Gc), sucrose (Glc-α1,2-Fru), maltose (Glc-α1,4-Glc) and melibiose (Gal-α1,6-Glc).

“Oligosaccharide” as the term is used herein and as generally understood in the state of the art, refers to a saccharide polymer containing a small number, typically three to twenty, preferably three to ten, of simple sugars, i.e., monosaccharides. The oligosaccharide as used in the disclosure can be a linear structure or can include branches. The linkage (e.g., glycosidic linkage, galactosidic linkage, glucosidic linkage, etc.) between two sugar units can be expressed, for example, as 1,4, 1->4, or (1-4), used interchangeably herein. For example, the terms “Gal-b1,4-Glc”, “Gal-β1,4-Glc”, “b-Gal-(1->4)-Glc”, “β-Gal-(1->4)-Glc”, “Galbeta1-4-Glc”, “Gal-β(1-4)-Glc” and “Gal-β(1-4)-Glc” have the same meaning, i.e., a beta-glycosidic bond links carbon-1 of galactose (Gal) with the carbon-4 of glucose (Glc). Each monosaccharide can be in the cyclic form (e.g., pyranose or furanose form). Linkages between the individual monosaccharide units may include alpha 1->2, alpha 1->3, alpha 1->4, alpha 1->6, alpha 2->1, alpha 2->3, alpha 2->4, alpha 2->6, beta 1->2, beta 1->3, beta 1->4, beta 1->6, beta 2->1, beta 2->3, beta 2->4, and beta 2->6. An oligosaccharide can contain both alpha- and beta-glycosidic bonds or can contain only alpha-glycosidic or only beta-glycosidic bonds. The term “polysaccharide” refers to a compound consisting of a large number, typically more than twenty, of monosaccharides linked glycosidically.

Examples of oligosaccharides include but are not limited to Lewis-type antigen oligosaccharides, mammalian (including human) milk oligosaccharides, O-antigen, enterobacterial common antigen (ECA), the glycan chain present in lipopolysaccharides (LPS), the oligosaccharide repeats present in capsular polysaccharides, peptidoglycan (PG), amino-sugars, antigens of the human ABO blood group system, an animal oligosaccharide, preferably selected from the list consisting of N-glycans and O-glycans, a plant oligosaccharide, preferably selected from the list consisting of N-glycans and O-glycans, sialylated oligosaccharide, neutral (non-charged) oligosaccharide, negatively charged oligosaccharide, fucosylated oligosaccharide, N-acetylglucosamine containing oligosaccharides, lacto-N-biose containing oligosaccharides, N-acetyllactosamine-containing oligosaccharides, N-acetylglucosamine containing sialylated oligosaccharides, N-acetylglucosamine containing neutral (non-charged) oligosaccharides, N-acetylglucosamine containing negatively charged oligosaccharides, N-acetylglucosamine containing fucosylated oligosaccharides, N-acetylglucosamine containing non-fucosylated oligosaccharides, lacto-N-biose containing sialylated oligosaccharides, lacto-N-biose containing neutral (non-charged) oligosaccharides, lacto-N-biose containing negatively charged oligosaccharides, lacto-N-biose containing fucosylated oligosaccharides, lacto-N-biose containing non-fucosylated oligosaccharides, N-acetyllactosamine containing sialylated oligosaccharides, N-acetyllactosamine containing neutral (non-charged) oligosaccharides, N-acetyllactosamine containing negatively charged oligosaccharides, N-acetyllactosamine containing fucosylated oligosaccharides, N-acetyllactosamine containing non-fucosylated oligosaccharides, chitosan, chitosan comprising oligosaccharide, heparosan, chondroitin sulphate, glycosaminoglycan oligosaccharide, heparin, heparan sulphate, dermatan sulphate, hyaluronan, hyaluronic acid and keratan sulphate.

The terms “negatively charged oligosaccharide” or “acidic oligosaccharide” are used interchangeably and refer to an oligosaccharide with a negative charge. In a preferred embodiment, the negatively charged oligosaccharide is a sialylated oligosaccharide. As used herein, a ‘sialylated oligosaccharide’ is to be understood as a negatively charged sialic acid containing oligosaccharide, i.e., an oligosaccharide having one or more sialic acid residue(s). It has an acidic nature. Such sialylated oligosaccharide is a saccharide structure comprising at least three monosaccharide subunits linked to each other via glycosidic bonds, wherein at least one of the monosaccharide subunit is a sialic acid residue as described herein. A sialylated oligosaccharide can contain more than one sialic acid residue, e.g., two, three or more. The more than one sialic acid residue can be two, three or more identical sialic acid residues. The more than one sialic acid residue can also be two, three or more different sialic acid residues. For example, a sialylated oligosaccharide can contain one or more Neu5Ac residues and one or more KDO residues. Some examples are 3-SL (3′-sialyllactose or 3′SL or Neu5Ac-α2,3-Gal-β1,4-Glc), 3′-sialyllactosamine, 6-SL (6′sialyllactose, 6′-sialyllactose or 6′SL or Neu5Ac-α2,6-Gal-β1,4-Glc), 3,6-disialyllactose (Neu5Ac-α2,3-(Neu5Ac-α2,6)-Gal-β1,4-Glc), 6,6′-disialyllactose (Neu5Ac-α2,6-Gal-β1,4-(Neu5Ac-α2,6)-Glc), 8,3-disialyllactose (Neu5Ac-α2,8-Neu5Ac-α2,3-Gal-β1,4-Glc), 6′-sialyllactosamine, oligosaccharides comprising 6′sialyllactose (also known as 6′sialyllactose, 6′SL and 6′-SL), SGG hexasaccharide (Neu5Aca-2,3Galβ-1,3GalNaco-1,3Galα-1,4Galβ-1,4Gal), sialylated tetrasaccharide, sialylated pentasaccharide, sialylated lacto-N-triose, sialylated lacto-N-tetraose, sialyllacto-N-neotetraose, LSTc (Neu5Ac-α2,6-Gal-β1,4-GlcNAc-β1,3-Gal-β1,4-Glc), LSTd (Neu5Ac-α2,3-Gal-β1,4-GlcNAc-β1,3-Gal-β1,4-Glc), monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a (LSTa, Neu5Ac-α2,3-Gal-β1,3-GlcNAc-β1,3-Gal-β1,4-Glc), disialyllacto-N-hexaose I, sialyllacto-N-tetraose b (LSTb, Gal-β1,3-(Neu5Ac-α2,6)-GlcNAc-β1,3-Gal-β1,4-Glc), 3′-sialyl-3-fucosyllactose, fucodisialyllacto-N-hexaose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose, Neu5Ac-a2,3-Gal-b1,4-GlcNAc-b1,3-Gal, Neu5Ac-a2,3-Gal-b1,3-GlcNAc-b1,3-Gal, 3′-KDO-lactose, 3′-KDO-lactosamine, 3′-KDO-lacto-N-biose, 3′-KDO-6′sialyllactose, 3′KDO-8-sialyllactose, KDO-2,3Galβ-1,3GalNaco-1,3Galα-1,4Galβ-1,4Gal, KDO-2,3Galβ-1,3GlcNacβ-1,3Galβ-1,4Glc, KDO-2,3Galβ-1,4GlcNacβ-1,3Galβ-1,4Glc, 3′-KDO-3-fucosyllactose, Neu5Ac-a2,8-Neu5Ac-a2,3-Gal-b1,3-GlcNAc-b1,3-Gal, 3′-Sialyl-2′-fucosyllactose, 6′-Sialyl-2′-fucosyllactose, 6′-Sialyl-3-fucosyllactose, Neu5Ac-a2,6-(Neu5Ac-a2,3-)Gal-b1,4-Glc, 3′-Sialyl-3-fucosyllactosamine, Fuc-a1,4-(Neu5Ac-a2,3-Gal-b1,3-)GlcNAc, 6′-Sialyllacto-N-biose, 3′-Sialyllacto-N-biose, Neu5Ac-a2,6-(GlcNAc-b1,3-)Gal-b1,4-Glc, Neu5Ac-a2,6-(Gal-b1,4-(Fuc-a1,3-)GlcNAc-b1,3-)Gal-b1,4-Glc, Neu5Ac-a2,3-Gal-b1,4-(Fuc-a1,3-)GlcNAc-b1,3-Gal-b1,4-Glc, Neu5Ac-a2,6-(Neu5Ac-a2,6-Gal-b1,4-(Fuc-a1,3-)GlcNAc-b1,3-)Gal-b1,4-Glc, Neu5Ac-a2,6-(Gal-b1,4-GlcNAc-b1,3-)Gal-b1,4-Glc, Neu5Ac-a2,6-(Gal-b1,3-GlcNAc-b1,3-)Gal-b1,4-(Fuc-a1,3-)Glc, Neu5Ac-a2,6-Gal-b1,3-GlcNAc-b1,3-Gal-b1,4-(Fuc-a1,3-)Glc, Neu5Ac-a2,3-Gal-b1,3-GlcNAc-b1,3-Gal-b1,4-(Fuc-a1,3-)Glc, Neu5Ac-a2,3-(Fuc-a1,2-)Gal-b1,3-GlcNAc-b1,3-Gal-b1,4-Glc, Neu5Ac-a2,6-(Fuc-a1,2-Gal-b1,3-GlcNAc-b1,3-)Gal-b1,4-Glc, Neu5Ac-a2,6-(Fuc-a1,2-)Gal-b1,3-GlcNAc-b1,3-Gal-b1,4-Glc, Fuc-a1,4-(Neu5Ac-a2,3-Gal-b1,3-)GlcNAc-b1,3-Gal-b1,4-Glc, Neu5Ac-a2,6-(Neu5Ac-a2,6-Gal-b1,3-GlcNAc-b1,3-)Gal-b1,4-(Fuc-a1,3-)Glc, Neu5Ac-a2,6-(Neu5Ac-a2,6-(Fuc-a1,2-)Gal-b1,3-GlcNAc-b1,3-)Gal-b1,4-Glc, Neu5Ac-a2,6-(Gal-b1,3-GlcNAc-b1,3-)Gal-b1,4-Glc, Neu5Ac-a2,6-Gal-b1,3-GlcNAc-b1,3-Gal-b1,4-Glc, Neu5Ac-a2,6-(Neu5Ac-a2,3)-Gal-b1,4-GlcNAc-b1,3-Gal-b1,4-Glc, Neu5Ac-a2,6-(Neu5Ac-a2,3-Gal-b1,3-GlcNAc-b1,3-)Gal-b1,4-Glc, Neu5Ac-a2,6-(Neu5Ac-a2,6-Gal-b1,3-GlcNAc-b1,3)-Gal-b1,4-Glc, Neu5Ac-a2,6-(Neu5Ac-a2,6-(Neu5Ac-a2,3)-Gal-b1,3-GlcNAc-b1,3)-Gal-b1,4-Glc, Neu5Ac-a2,6-(Neu5Ac-a2,3-Gal-b1,4-GlcNAc-b1,3)-Gal-b1,4-Glc, Neu5Ac-a2,6-(Neu5Ac-a2,6-Gal-b1,4-GlcNAc-b1,3)-Gal-b1,4-Glc, Neu5Ac-a2,6-(Neu5Ac-a2,6-(Neu5Ac-a2,3)-Gal-b1,4-GlcNAc-b1,3)-Gal-b1,4-Glc, Neu5Ac-a2,6-(Gal-b1,4-GlcNAc-b1,3-Gal-b1,4-GlcNAc-b1,3)-Gal-b1,4-Glc, Neu5Ac-a2,6-(Gal-b1,4-GlcNAc-b1,3)-Gal-b1,4-GlcNAc-b1,3-Gal-b1,4-Glc, Neu5Ac-a2,6-Gal-b1,4-GlcNAc-b1,3-Gal-b1,4-GlcNAc-b1,3-Gal-b1,4-Glc, Neu5Ac-a2,6-(Neu5Ac-a2,6-(Neu5Ac-a2,6-Gal-b1,4-GlcNAc-b1,3)-Gal-b1,4-GlcNAc-b1,3)-Gal-b1,4-Glc, Neu5Ac-a2,3-Gal-b1,4-GlcNAc-b1,3-Gal-b1,4-GlcNAc-b1,3-Gal-b1,4-Glc, 6′-KDO-lactose, 6′-KDO-lactosamine, 6′-KDO-lacto-N-biose, KDO-lacto-N-triose, KDO-lacto-N-tetraose, KDO-lacto-N-tetraose, and oligosaccharides bearing one or several sialic acid residue(s), including but not limited to: oligosaccharide moieties of the gangliosides selected from GM3 (3′sialyllactose, Neu5Aca-2,3Galβ-4Glc) and oligosaccharides comprising the GM3 motif, GD3 Neu5Aca-2,8Neu5Aca-2,3Galβ-1,4Glc GT3 (Neu5Aca-2,8Neu5Aca-2,8Neu5Aca-2,3Galβ-1,4Glc); GM2 GalNAcα-1,4(Neu5Acα-2,3)Galβ-1,4Glc, GM1 Galβ-1,3GalNAcβ-1,4(Neu5Acα-2,3)Galβ-1,4Glc, GD1a Neu5Aca-2,3Galβ-1,3GalNAcβ-1,4(Neu5Aca-2,3)Galβ-1,4Glc, GT1a Neu5Aca-2,8Neu5Aca-2,3Galp-1,3GalNAcβ-1,4(Neu5Acα-2,3)Galβ-1,4Glc, GD2 GalNAcβ-1,4(Neu5Aca-2,8Neu5Acα2,3)Galβ-1,4Glc, GT2 GalNAcβ-1,4(Neu5Aca-2,8Neu5Aca-2,8Neu5Acα2,3)Galβ-1,4Glc, GD1b, Galβ-1,3GalNAcβ-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3Galβ-1,3GalNAc 3-1,4(Neu5Acα-2,8Neu5Acα2,3)Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAcβ-1,4(Neu5Acα-2,8Neu5Acα-2,8Neu5Acα2,3)Galβ-1,4Glc, GP1c Neu5Acα-2,8Neu5Acα-2,3Galβ-1,3GalNAcβ-1,4(Neu5Acα-2,8Neu5Acα-2,8Neu5Acα2,3)Galβ-1,4Glc, GD1a Neu5Acα-2,3Galβ-1,3(Neu5Acα-2,6)GalNAcβ-1,4Galβ-1,4Glc, Fucosyl-GM1 Fucα-1,2Galβ-1,3GalNAcβ-1,4(Neu5Acα-2,3)Galβ-1,4Glc; all of which may be extended to the production of the corresponding gangliosides by reacting the above oligosaccharide moieties with ceramide or synthetizing the above oligosaccharides on a ceramide.

“Charged oligosaccharides” are oligosaccharide structures that contain one or more negatively charged monosaccharide subunits including sialic acid, glucuronate and galacturonate. Charged oligosaccharides are also referred to as acidic oligosaccharides. In contrast, neutral (non-charged) oligosaccharides are non-sialylated oligosaccharides, and thus do not contain an acidic monosaccharide subunit. Neutral oligosaccharides comprise non-charged fucosylated oligosaccharides that contain one or more fucose subunits in their glycan structure as well as non-charged non-fucosylated oligosaccharides that lack any fucose subunit. Other examples of charged oligosaccharides are sulphated chitosans and deacetylated chitosans.

The terms “3′ sialyllactose”, “3′-sialyllactose”, “alpha-2,3-sialyllactose”, “alpha 2,3 sialyllactose”, “α-2,3-sialyllactose”, “a 2,3 sialyllactose”, “3SL” or “3′SL” as used in the disclosure, are used interchangeably and refer to the product obtained by the catalysis of the alpha-2,3-fucosyltransferase transferring the sialic acid group from CMP-Neu5Ac to lactose in an alpha-2,3-linkage. The terms “6′ sialyllactose”, “6′-sialyllactose”, “alpha-2,6-sialyllactose”, “alpha 2,6 sialyllactose”, “α-2,6-sialyllactose”, “a 2,6 sialyllactose”, “6SL” or “6′SL” as used in the disclosure, are used interchangeably and refer to the product obtained by the catalysis of the alpha-2,6-fucosyltransferase transferring the sialic acid group from CMP-Neu5Ac to lactose in an alpha-2,6-linkage. The terms “LSTa”, “LS-Tetrasaccharide a”, “Sialyl-lacto-N-tetraose a”, “sialyllacto-N-tetraose a” or “Neu5Ac-α2,3-Gal-β1,3-GlcNAc-β1,3-Gal-β1,4-Glc” as used in the disclosure, are used interchangeably. The terms “LSTb”, “LS-Tetrasaccharide b”, “Sialyl-lacto-N-tetraose b”, “sialyllacto-N-tetraose b” or “Gal-β1,3-(Neu5Ac-α2,6)-GlcNAc-β1,3-Gal-β1,4-Glc” as used in the disclosure, are used interchangeably. The terms “LSTc”, “LS-Tetrasaccharide c”, “Sialyl-lacto-N-tetraose c”, “sialyllacto-N-tetraose c”, “sialyllacto-N-neotetraose c” or “Neu5Ac-α2,6-Gal-β1,4-GlcNAc-β1,3-Gal-β1,4-Glc” as used in the disclosure, are used interchangeably. The terms “LSTd”, “LS-Tetrasaccharide d”, “Sialyl-lacto-N-tetraose d”, “sialyllacto-N-tetraose d”, “sialyllacto-N-neotetraose d” or “Neu5Ac-α2,3-Gal-β1,4-GlcNAc-β1,3-Gal-β1,4-Glc” as used in the disclosure, are used interchangeably. The terms “DSLNnT” and “Disialyllacto-N-neotetraose” are used interchangeably and refer to Neu5Ac-α2,6-Gal-β1,4-GlcNAc-β1,3-[Neu5Ac-α2,6]-Gal-β1,4-Glc. The terms “DSLNT”, “DS-LNT” and “Disialyllacto-N-tetraose” are used interchangeably and refer to Neu5Ac-α2,6-(Neu5Ac-α2,3-Gal-β1,3)-GlcNAc-β1,3-Gal-β1,4-Glc.

The terms ‘neutral oligosaccharide’ and ‘non-charged’ oligosaccharide as used herein are used interchangeably and refer, as generally understood in the state of the art, to an oligosaccharide that has no negative charge originating from a carboxylic acid group. Examples of such neutral oligosaccharide are 2′-fucosyllactose (2′FL), 3-fucosyllactose (3FL), 4-fucosyllactose (4FL), 6-fucosyllactose (6FL), 2′, 3-difucosyllactose (diFL), lacto-N-triose II (LN3), lacto-N-tetraose (LNT), lacto-N-neotetraose (LNnT), lacto-N-fucopentaose I (LNFP I), lacto-N-neofucopentaose I (LNnFP I), lacto-N-fucopentaose II (LNFP II), lacto-N-fucopentaose III (LNFP III), lacto-N-fucopentaose V (LNFP V), lacto-N-fucopentaose VI, lacto-N-neofucopentaose V (LNnFP V), lacto-N-difucohexaose I (LNDFH I), lacto-N-difucohexaose II (LNDFHII), Fuc-a1,2-Gal-b1,3-GlcNAc-b1,3-Gal-b1,4-(Fuc-a1,3-)Glc, Fuc-a1,2-Gal-b1,4-GlcNAc-b1,3-Gal-b1,4-(Fuc-a1,3-)Glc, Fuc-a1,2-Gal-b1,4-(Fuc-a1,3-)GlcNAc-b1,3-Gal-b1,4-Glc, Gal-b1,4-(Fuc-a1,3-)GlcNAc-b1,3-Gal-b1,4-(Fuc-a1,3-)Glc, Fuc-a1,2-Gal-b1,4-(Fuc-a1,3-)GlcNAc-b1,3-Gal-b1,4-(Fuc-a1,3-)Glc, Fuc-a1,4-(Fuc-a1,2-Gal-b1,3-)GlcNAc-b1,3-Gal-b1,4-(Fuc-a1,3-)Glc, monofucosyllacto-N-hexaose-III, difucosyllacto-N-hexaose (a), 6′-galactosyllactose, 3′-galactosyllactose, lacto-N-hexaose, lacto-N-neohexaose, para-lacto-N-hexaose, para-lacto-N-neohexaose, fucosyl-lacto-N-hexaose, difucosyl-lacto-N-hexaose, difucosyl-lacto-N-neohexaose (LNnDFH II), difucosyl-para-lacto-N-neohexaose, trifucosyllacto-N-hexaose, para-lacto-N-fucohexaose, lacto-N-trifucoheptaose, a1,3-galactosyl-3-fucosyllactose, Gal-a1,3-(Fuc-a1,2-)Gal-b1,4-(Fuc-a1,3-)Glc, GalNAc-a1,3-(Fuc-a1,2-)Gal-b1,4-(Fuc-a1,3-)Glc, 2-fucosyllactulose, 3-fucosyl-N-acetyllactosamine, 2′-fucosyl-N-acetyllactosamine, difucosyl-N-acetyllactosamine, 4-fucosyllacto-N-biose, 2′-fucosyllacto-N-biose, difucosyllacto-N-biose, GlcNAc-b1,3-Gal-b1,4-(Fuc-a1,3-)Glc, GlcNAc-b1,6-(GlcNAc-b1,3-)Gal-b1,4-Glc, lacto-N-pentaose (LN5), lacto-N-neopentaose, para lacto-N-pentaose, para lacto-N-neopentaose, lacto-N-novopentaose I, lacto-N-heptaose (LN7), lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose (pLNnO), iso lacto-N-nonaose, novo lacto-N-nonaose, lacto-N-nonaose (LN9), lacto-N-decaose, iso lacto-N-decaose, novo lacto-N-decaose, lacto-N-neodecaose, para lacto-N-neodecaose (pLNnD), a1,3-galactosyllacto-N-neotetraose, GlcNAc-b1,3-Gal-b1,3-GlcNAc-b1,3-Gal-b1,4-Glc, GlcNAc-b1,6-(Gal-b1,4-GlcNAc-b1,3-)Gal-b1,4-Glc and GlcNAc-b1,6-(Gal-b1,3-GlcNAc-b1,3-)Gal-b1,4-Glc.

The terms “LNT II”, “LNT-II”, “LN3”, “lacto-N-triose II”, “lacto-N-triose II”, “lacto-N-triose”, “lacto-N-triose” or “GlcNAcβ1-3Galβ1-4Glc” as used in the disclosure, are used interchangeably. The terms “LNT”, “lacto-N-tetraose”, “lacto-N-tetraose” or “Galβ1-3GlcNAcβ1-3Galβ1-4Glc” as used in the disclosure, are used interchangeably. The terms “LNnT”, “lacto-N-neotetraose”, “lacto-N-neotetraose”, “neo-LNT” or “Galβ1-4GlcNAcβ1-3Galβ1-4Glc” as used in the disclosure, are used interchangeably. The terms “LNB” and “Lacto-N-biose” are used interchangeably and refer to the disaccharide Gal-b1,3-GlcNAc. The terms “LacNAc” and “N-acetyllactosamine” are used interchangeably and refer to the disaccharide Gal-b1,4-GlcNAc.

A ‘fucosylated oligosaccharide’ as used herein and as generally understood in the state of the art is an oligosaccharide that is carrying a fucose-residue. Such fucosylated oligosaccharide is a saccharide structure comprising at least three monosaccharide subunits linked to each other via glycosidic bonds, wherein at least one of the monosaccharide subunit is a fucose. A fucosylated oligosaccharide can contain more than one fucose residue, e.g., two, three or more. A fucosylated oligosaccharide can be a neutral oligosaccharide or a charged oligosaccharide e.g., also comprising sialic acid structures. Fucose can be linked to other monosaccharide subunits comprising glucose, galactose, GlcNAc via alpha-glycosidic bonds comprising alpha-1,2 alpha-1,3, alpha-1,4, alpha-1,6 linkages.

As used herein, the term “O-antigen” refers to the repetitive glycan component of the surface lipopolysaccharide (LPS) of Gram-negative bacteria. The term “lipopolysaccharide” or “LPS” refers to glycolipids found in the outer membrane of Gram-negative bacteria, which are composed of a lipid A, a core oligosaccharide and the O-antigen. The term “capsular polysaccharides” refers to long-chain polysaccharides with oligosaccharide repeat structures that are present in bacterial capsules, the latter being a polysaccharide layer that lies outside the cell envelope. The terms “peptidoglycan” or “murein” refers to an essential structural element in the cell wall of most bacteria, being composed of sugars and amino acids, wherein the sugar components consist of alternating residues of beta-1,4 linked GlcNAc and N-acetylmuramic acid. The term “amino-sugar” as used herein refers to a sugar molecule in which a hydroxyl group has been replaced with an amine group. As used herein, an antigen of the human ABO blood group system is an oligosaccharide. Such antigens of the human ABO blood group system are not restricted to human structures. The structures involve the A determinant GalNAc-alpha1,3(Fuc-alpha1,2)-Gal-, the B determinant Gal-alpha1,3(Fuc-alpha1,2)-Gal- and the H determinant Fuc-alpha1,2-Gal- that are present on disaccharide core structures comprising Gal-beta1,3-GlcNAc, Gal-beta1,4-GlcNAc, Gal-beta1,3-GalNAc and Gal-beta1,4-Glc.

Mammalian milk oligosaccharides comprise oligosaccharides present in milk found in any phase during lactation including colostrum milk from humans (i.e., human milk oligosaccharides or HMOs) and mammals including but not limited to cows (Bos Taurus), sheep (Ovis aries), goats (Capra aegagrus hircus), bactrian camels (Camelus bactrianus), horses (Equusferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus Vulpecula), koalas (Phascolarctos cinereus), eastern quolls (Dasyurus viverrinus), platypus (Ornithorhynchus anatinus). As used herein, “mammalian milk oligosaccharide” or “MMO” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharides and/or sialylated oligosaccharides.

The terms “human milk oligosaccharide” or “HMO” refer to oligosaccharides found in human breast milk, including preterm human milk, colostrum and term human milk. HMOs comprise fucosylated oligosaccharides, non-fucosylated neutral oligosaccharides and sialylated oligosaccharides (see e.g., Chen X., Chapter Four: Human Milk Oligosaccharides (HMOS): Structure, Function, and Enzyme-Catalyzed Synthesis in Adv. Carbohydr. Chem. Biochem. 72, 113 (2015)). Examples of HMOs comprise 3-fucosyllactose, 2′-fucosyllactose, 2′,3-difucosyllactose, 6′-sialyllactose, 3′-sialyllactose, LN3, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, difucosyllacto-N-tetraose, lacto-N-hexaose, lacto-N-difucohexaose I, lacto-N-difucohexaose II, disialyllacto-N-tetraose, fucosyllacto-N-hexaose, difucosyllacto-N-hexaose, fucodisialyllacto-N-hexaose, disialyllacto-N-hexaose.

As used herein, the term “mammary cell(s)” generally refers to mammalian mammary epithelial cell(s), mammalian mammary-epithelial luminal cell(s), or mammalian epithelial alveolar cell(s), or any combination thereof. As used herein, the term “mammary-like cell(s)” generally refers to mammalian cell(s) having a phenotype/genotype similar (or substantially similar) to natural mammalian mammary cell(s) but is/are derived from mammalian non-mammary cell source(s). Such mammalian mammary-like cell(s) may be engineered to remove at least one undesired genetic component and/or to include at least one predetermined genetic construct that is typical of a mammalian mammary cell. Non-limiting examples of mammalian mammary-like cell(s) may include mammalian mammary epithelial-like cell(s), mammalian mammary epithelial luminal-like cell(s), mammalian non-mammary cell(s) that exhibits one or more characteristics of a cell of a mammalian mammary cell lineage, or any combination thereof. Further non-limiting examples of mammalian mammary-like cell(s) may include mammalian cell(s) having a phenotype similar (or substantially similar) to natural mammalian mammary cell(s), or more particularly a phenotype similar (or substantially similar) to natural mammalian mammary epithelial cell(s). A mammalian cell with a phenotype or that exhibits at least one characteristic similar to (or substantially similar to) a natural mammalian mammary cell or a mammalian mammary epithelial cell may comprise a mammalian cell (e.g., derived from a mammary cell lineage or a non-mammary cell lineage) that exhibits either naturally, or has been engineered to, be capable of expressing at least one milk component.

As used herein, the term “non-mammary cell(s)” may generally include any mammalian cell of non-mammary lineage. In the context of the disclosure, a non-mammary cell can be any mammalian cell capable of being engineered to express at least one milk component. Non-limiting examples of such non-mammary cell(s) include hepatocyte(s), blood cell(s), kidney cell(s), cord blood cell(s), epithelial cell(s), epidermal cell(s), myocyte(s), fibroblast(s), mesenchymal cell(s), or any combination thereof. In some instances, molecular biology and genome editing techniques can be engineered to eliminate, silence, or attenuate myriad genes simultaneously.

The terms “hydrolyzing UDP-N-acetyl-D-glucosamine-2-epimerase”, “hydrolyzing UDP-N-acylglucosamine 2-epimerase”, “hydrolyzing UDP-GlcNAc-2-epimerase”, “hydrolyzing UDP-N-acetyl-D-glucosamine 2-epimerase”, “hydrolyzing UDP-N-acetylglucosamine-2-epimerase”, “hydrolyzing UDP-N-acetylglucosamine 2-epimerase”, “hydrolyzing uridine diphosphate-N-acetylglucosamine-2′-epimerase” and “hydrolyzing uridine diphosphoacetylglucosamine 2′-epimerase” are used interchangeably and refer to an enzyme that catalyzes the reaction UDP-N-acetyl-D-glucosamine (UDP-GlcNAc)=N-acetylmannosamine (ManNAc). The nnaA polypeptide is an example of a hydrolyzing UDP-N-acetyl-D-glucosamine-2-epimerase.

The term “non-hydrolyzing UDP-N-acetyl-D-glucosamine-2-epimerase” refers to an enzyme that catalyzes the reaction UDP-N-acetyl-D-glucosamine (UDP-GlcNAc)=UDP-N-acetylmannosamine (UDP-ManNAc).

A ‘sialylation pathway’ is a biochemical pathway consisting of at least one of the enzymes and their respective genes selected from the list comprising an L-glutamine-D-fructose-6-phosphate aminotransferase, a phosphoglucosamine mutase, an N-acetylglucosamine-6-P deacetylase, an N-acylglucosamine 2-epimerase, a UDP-N-acetylglucosamine 2-epimerase, an N-acetylmannosamine-6-phosphate 2-epimerase, a UDP-GlcNAc 2-epimerase/kinase, a glucosamine 6-phosphate N-acetyltransferase, an N-acetylglucosamine-6-phosphate phosphatase, a phosphoacetylglucosamine mutase, an N-acetylglucosamine 1-phosphate uridylyltransferase, a glucosamine-1-phosphate acetyltransferase, a sialic acid synthase, an N-acetylneuraminate lyase, an N-acylneuraminate-9-phosphate synthase, an N-acylneuraminate-9-phosphatase, a d-arabinose 5-phosphate isomerase, a KDO-8P synthase, a KDO 8-phosphate phosphatase, a CMP-KDO synthetase, a sialic acid transporter and a CMP-sialic acid synthase, combined with a sialyltransferase leading to a 2,3; a 2,6 and/or a 2,8 sialylated oligosaccharides.

Said pathway for production of a negatively charged, preferably sialylated, oligosaccharide may comprise a pathway for synthesis and/or import of a co-factor and/or a precursor used in the pathway for production of the negatively charged, preferably sialylated, oligosaccharide.

The term “reductive pathway” as used herein is a biochemical pathway that results in the cytoplasmic environment of the cell to be reducing, wherein the reducing environment negatively influences proper protein folding and blocks disulfide bond formation. Examples of a reductive pathway comprise but are not limited to the reductive acetyl-CoA-pathway, the reductive pyrimidine catabolic pathway, the reductive citric acid cycle, the thiol-redox pathway and the reductive glycine pathway. A reducing cytoplasm means e.g., that the NADP+:NADPH ratio in the cytoplasmic environment is low and/or that the glutathione (GSH) levels are high (e.g., 10 mM). Reducing environments may affect the oxidation state of a molecule, thereby altering its solubility.

The terms “glutathione reductase”, “glutathione reductase (NADPH)”, “glutathione S-reductase”, “GSH reductase”, “GSSG reductase”, “NADPH:oxidized-glutathione oxidoreductase” “NADPH-glutathione reductase”, “NADPH-GSSG reductase”, “gor”, “GR” and “GRase” are used interchangeably and refer to an enzyme that catalyzes the reaction 2 glutathione+NADP+=glutathione disulfide+H++NADPH.

The terms “thioredoxin reductase”, “thioredoxin-disulfide reductase”, “NADPH:oxidized thioredoxin oxidoreductase”, “NADPH-thioredoxin reductase”, “NADP-thioredoxin reductase”, “thioredoxin reductase (NADPH)”, “trxB” and “TRXR”, are used interchangeably and refer to an enzyme that catalyzes the reaction [thioredoxin]-dithiol+NADP+=[thioredoxin]-disulfide+H++NADPH.

The terms “disulfide bond isomerase”, “protein disulfide-isomerase”, “S—S rearrangase” and “PDI” are used interchangeably and refer to an enzyme that catalyzes the rearrangement of disulfide bonds in proteins.

The terms “disulfide oxidoreductase”, “disulfide oxidoreductase 2”, “thiol:disulfide oxidoreductase”, “dsbC” and “xprA” are used interchangeably and refer to an enzyme that is reported to be involved in protein disulfide bond rearrangement and/or formation.

The terms “thiol oxidase” and “sulfhydryl oxidase” are used interchangeably and refer to an enzyme that catalyzes the reaction O2+2R′C(R)SH=H2O2+R′C(R)S—S(R)CR′.

The term “chaperone” refers to an enzyme that assist in protein folding. Examples are PDI, SecB, ERp57, heat shock proteins or Hsps, such as e.g., Hsp10, Hsp60, Hsp70, Hsp90. The terms “pyruvate dehydrogenase”, “pyruvate oxidase”, “POX”, “poxB” and “pyruvate:ubiquinone-8 oxidoreductase” are used interchangeably and refer to an enzyme that catalyzes the oxidative decarboxylation of pyruvate to produce acetate and CO2.

The terms “lactate dehydrogenase”, “D-lactate dehydrogenase”, “ldhA”, “hslI”, “htpH”, “D-LDH”, “fermentative lactate dehydrogenase” and “D-specific 2-hydroxyacid dehydrogenase” are used interchangeably and refer to an enzyme that catalyzes the conversion of lactate into pyruvate hereby generating NADH.

The term “membrane transporter proteins” as used herein refers to proteins that are part of or interact with the cell membrane and control the flow of molecules and information across the cell. The membrane proteins are thus involved in transport, be it import into or export out of the cell. Such membrane transporter proteins can be but are not limited to porters, P—P-bond-hydrolysis-driven transporters, β-Barrel Porins, auxiliary transport proteins and phosphotransfer-driven group translocators (Forrest et al., Biochim. Biophys. Acta 1807 (2011) 167-188; Lengeler, J. Mol. Microbiol. Biotechnol. 25 (2015) 79-93; Moraes and Reithmeier, Biochim. Biophys. Acta 1818 (2012), 2687-2706; Saier et al., Nucleic Acids Res. 44 (2016) D372-D379).

The term “Siderophore” as used herein is referring to the secondary metabolite of various microorganisms, which are mainly ferric ion specific chelators. These molecules have been classified as catecholate, hydroxamate, carboxylate and mixed types. Siderophores are in general synthesized by a nonibosomal peptide synthetase (NRPS) dependent pathway or an NRPS independent pathway (NIS). The most important precursor in NRPS-dependent siderophore biosynthetic pathway is chorismate. 2, 3-DHBA could be formed from chorismate by a three-step reaction catalyzed by isochorismate synthase, isochorismatase, and 2, 3-dihydroxybenzoate-2, 3-dehydrogenase. Siderophores can also be formed from salicylate, which is formed from isochorismate by isochorismate pyruvate lyase. When ornithine is used as precursor for siderophores, biosynthesis depends on the hydroxylation of ornithine catalyzed by L-ornithine N5-monooxygenase. In the NIS pathway, an important step in siderophore biosynthesis is N(6)-hydroxylysine synthase. A transporter is needed to export the siderophore outside the cell. Four superfamilies of membrane proteins are identified so far in this process: the major facilitator superfamily (MFS); the Multidrug/Oligosaccharidyl-lipid/Polysaccharide Flippase Superfamily (MOP); the resistance, nodulation and cell division superfamily (RND); and the ABC superfamily. In general, the genes involved in siderophore export are clustered together with the siderophore biosynthesis genes. The term “siderophore exporter” as used herein refers to such transporters needed to export the siderophore outside of the cell.

The ATP-binding cassette (ABC) superfamily contains both uptake and efflux transport systems, and the members of these two porter groups generally cluster loosely together. ATP hydrolysis without protein phosphorylation energizes transport. There are dozens of families within the ABC superfamily, and family generally correlates with substrate specificity. Members are classified according to class 3.A.1 as defined by the Transporter Classification Database operated by the Saier Lab Bioinformatics Group available via www.tcdb.org and providing a functional and phylogenetic classification of membrane transporter proteins.

The major facilitator superfamily (MFS) is a superfamily of membrane transporter proteins catalyzing uniport, solute:cation (H+, but seldom Na+) symport and/or solute:H+ or solute:solute antiport. Most are of 400-600 amino acyl residues in length and possess either 12, 14, or occasionally, 24 transmembrane α-helical spanners (TMSs) as defined by the Transporter Classification Database operated by the Saier Lab Bioinformatics Group (www.tcdb.org).

“SET” or “Sugar Efflux Transporter” as used herein refers to membrane proteins of the SET family, which are proteins with InterPRO domain IPR004750 and/or are proteins that belong to the eggNOGv4.5 family ENOG410XTE9. Identification of the InterPro domain can be done by using the online tool on https://www.ebi.ac.uk/interpro/ or a standalone version of InterProScan (https://www.ebi.ac.uk/interpro/download.html) using the default values. Identification of the orthology family in eggNOGv4.5 can be done using the online version or a standalone version of eggNOG-mapperv1 (http://eggnogdb.embl.de/#/app/home).

The term “enabled efflux” means to introduce the activity of transport of a solute over the cytoplasm membrane and/or the cell wall. The transport may be enabled by introducing and/or increasing the expression of a membrane transporter protein as described in the disclosure. The term “enhanced efflux” means to improve the activity of transport of a solute over the cytoplasm membrane and/or the cell wall. Transport of a solute over the cytoplasm membrane and/or cell wall may be enhanced by introducing and/or increasing the expression of a membrane transporter protein as described in the disclosure. “Expression” of a membrane transporter protein is defined as “overexpression” of the gene encoding the membrane transporter protein in the case the gene is an endogenous gene or “expression” in the case the gene encoding the membrane transporter protein is a heterologous gene that is not present in the wild-type strain or cell.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50%, 55%, 60%, 65%, 70%, 75%, 80% or 85% pure, usually at least about 90%, 91%, 92%, 93%, 94%, 95%, 96%, 97%, 98%, or 99.0% pure as measured by band intensity on a silver-stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For di- and oligosaccharides, purity can be determined using methods such as but not limited to thin layer chromatography, gas chromatography, NMR, HPLC, capillary electrophoresis or mass spectroscopy. Further herein, the terms “contaminants” and “impurities” preferably mean particulates, cells, cell components, metabolites, cell debris, proteins, peptides, amino acids, nucleic acids, glycolipids and/or endotoxins, which can be present in an aqueous medium like e.g., a cultivation or an incubation.

The term “clarifying” as used herein refers to the act of treating an aqueous medium like e.g., a cultivation or an incubation, to remove suspended particulates and contaminants from the production process, like e.g., cells, cell components, insoluble metabolites and debris, that could interfere with the eventual purification of the negatively charged, preferably sialylated, oligosaccharide. Such treatment can be carried out in a conventional manner by centrifugation, flocculation, flocculation with optional ultrasonic treatment, gravity filtration, microfiltration, foam separation or vacuum filtration (e.g., through a ceramic filter, which can include a Celite™ filter aid).

The term “cultivation” refers to the culture medium wherein the cell is cultivated, or fermented, the cell itself, and a negatively charged, preferably sialylated, oligosaccharide that is produced by the cell in whole broth, i.e., inside (intracellularly) as well as outside (extracellularly) of the cell. The terms “culture medium” and “cultivation medium” as used herein are used interchangeably and refer to the medium wherein the cell is cultivated.

The term “incubation” refers to a mixture wherein a negatively charged, preferably sialylated, oligosaccharide is produced. The mixture can comprise one or more enzyme(s), one or more precursor(s) and one or more acceptor(s) as defined herein present in a buffered solution and incubated for a certain time at a certain temperature enabling production of a negatively charged, preferably sialylated, oligosaccharide, catalyzed by the one or more enzyme(s) using the one or more precursor(s) and the one or more acceptor(s) in the mixture. The mixture can also comprise i) the cell obtained after cultivation or incubation, optionally the cell is subjected to cell lysis, ii) a buffered solution or the cultivation or incubation medium wherein the cell was cultivated or fermented, and iii) a negatively charged, preferably sialylated, oligosaccharide that is produced by the cell in whole broth, i.e., inside (intracellularly) as well as outside (extracellularly) of the cell. The incubation can also be the cultivation as defined herein.

The terms “reactor” and “incubator” refer to the recipient filled with the cultivation or incubation. Examples of reactors and incubators comprise but are not limited to microfluidic devices, well plates, tubes, shake flasks, fermenters, bioreactors, process vessels, cell culture incubators, CO2 incubators.

As used herein, the term “cell productivity index (CPI)” refers to the mass of the negatively charged, preferably sialylated, oligosaccharide produced by the cells divided by the mass of the cells produced in the culture.

The term “precursor” as used herein refers to substances that are taken up or synthetized by the cell for the specific production of a negatively charged, preferably sialylated, oligosaccharide according to the disclosure. In this sense a precursor can be an acceptor as defined herein, but can also be another substance, metabolite, co-factor, which is first modified within the cell as part of the biochemical synthesis route of a negatively charged, preferably sialylated, oligosaccharide. The term “precursor” as used herein is also to be understood as a chemical compound that participates in an incubation, a chemical or an enzymatic reaction to produce another compound like e.g., an intermediate or an acceptor as defined herein, as part in the metabolic pathway of a negatively charged, preferably sialylated, oligosaccharide. The term “precursor” as used herein is also to be understood as a donor that is used by a glycosyltransferase to modify an acceptor as defined herein with a sugar moiety in a glycosidic bond, as part in the metabolic pathway of a negatively charged, preferably sialylated, oligosaccharide. Examples of such precursors comprise the acceptors as defined herein, and/or dihydroxyacetone, glucosamine, N-acetylglucosamine, N-acetylmannosamine, galactosamine, N-acetylgalactosamine, galactosyllactose, phosphorylated sugars or sugar phosphates like e.g., but not limited to glucose-1-phosphate, galactose-1-phosphate, glucose-6-phosphate, fructose-6-phosphate, fructose-1,6-bisphosphate, mannose-6-phosphate, mannose-1-phosphate, glycerol-3-phosphate, glyceraldehyde-3-phosphate, dihydroxyacetone-phosphate, glucosamine-6-phosphate, N-acetylglucosamine-6-phosphate, N-acetylmannosamine-6-phosphate, N-acetylglucosamine-1-phosphate, N-acetylneuraminic acid-9-phosphate and nucleotide-activated sugars like nucleotide diphospho-sugars and nucleotide monophospho-sugars as defined herein like e.g., UDP-glucose, UDP-galactose, UDP-N-acetylglucosamine, CMP-sialic acid, GDP-mannose, GDP-4-dehydro-6-deoxy-α-D-mannose, GDP-fucose.

Optionally, the cell used to produce the negatively charged, preferably sialylated, oligosaccharide is transformed to comprise and to express at least one nucleic acid sequence encoding a protein selected from the list consisting of lactose transporter, N-acetylneuraminic acid transporter, fucose transporter, glucose transporter, galactose transporter, transporter for a nucleotide-activated sugar wherein the transporter internalizes a to the medium added precursor for the synthesis of the negatively charged, preferably sialylated, oligosaccharide of disclosure.

The term “acceptor” as used herein refers to a mono-, di- or oligosaccharide, which can be modified by a glycosyltransferase. Examples of such acceptors comprise glucose, galactose, fructose, glycerol, sialic acid, fucose, mannose, N-acetylglucosamine, maltose, sucrose, lactose, lactulose, lactobionic acid (LBA), N-acetyllactosamine, lacto-N-biose, lacto-N-triose, lacto-N-tetraose (LNT), lacto-N-neotetraose (LNnT), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, LSTa, LSTb, LSTc and oligosaccharide containing 1 or more N-acetyllactosamine units and/or 1 or more lacto-N-biose units or an intermediate into oligosaccharide, fucosylated and sialylated versions thereof, ceramide, N-acylated sphingoid, glucosylceramide, lactosylceramide, sphingosine, phytosphingosine, sphingosine synthons, peptide backbones with beta-GlcNAc-Asn residues, glycoproteins with terminal GlcNAc and Gal residues, immunoglobulins.

### DETAILED DESCRIPTION

According to a first aspect, the disclosure provides a cell that comprises a pathway for production of a negatively charged, preferably sialylated, oligosaccharide, the cell being genetically engineered to possess or express, preferably to over-express, a hydrolyzing UDP-N-acetyl-D-glucosamine-2-epimerase wherein the hydrolyzing UDP-N-acetyl-D-glucosamine-2-epimerase comprises an amino acid sequence comprising a conserved motif with SEQ ID NO: 12 and having hydrolyzing UDP-N-acetyl-D-glucosamine-2-epimerase activity.

According to a second aspect, the disclosure provides a method for the production of a negatively charged, preferably sialylated, oligosaccharide, wherein the method comprises cultivating and/or incubating a cell as described herein, in cultivation and/or incubation medium under conditions permissive to produce a negatively charged, preferably sialylated, oligosaccharide.

In the scope of the disclosure, permissive conditions are understood to be conditions relating to physical or chemical parameters including but not limited to temperature, pH, pressure, osmotic pressure and product/precursor/acceptor concentration.

In a particular embodiment, the permissive conditions may include a temperature-range of 30+/−20 degrees centigrade, a pH-range of 7+/−3.

In a preferred embodiment, the negatively charged, preferably sialylated, oligosaccharide is separated from the cultivation and/or incubation. In another and/or additional preferred embodiment, the oligosaccharide is purified from the cultivation and/or incubation.

In a specific embodiment, the cell comprises a pathway for production of a negatively charged, preferably sialylated, oligosaccharide. The term “pathway for production of a negatively charged, preferably sialylated, oligosaccharide” as used herein is a biochemical pathway consisting of the enzymes and their respective genes involved in the synthesis of a negatively charged, preferably sialylated, oligosaccharide as defined herein. The pathway for production of a negatively charged, preferably sialylated, oligosaccharide can comprise but is not limited to pathways involved in the synthesis of a nucleotide-activated sugar and the transfer of the nucleotide-activated sugar to an acceptor to create a negatively charged, preferably sialylated, oligosaccharide of the disclosure. Examples of such pathways comprise but are not limited to a fucosylation pathway, a sialylation pathway, a galactosylation pathway, an N-acetylglucosaminylation pathway, an N-acetylgalactosaminylation pathway, a mannosylation pathway and an N-acetylmannosaminylation pathway.

In another additional specific embodiment, the cell is genetically engineered to possess or express a hydrolyzing UDP-N-acetyl-D-glucosamine-2-epimerase as described herein. In a preferred embodiment, the cell is genetically engineered to over-express a hydrolyzing UDP-N-acetyl-D-glucosamine-2-epimerase as described herein.

In a preferred aspect of the method and/or cell of the disclosure, the genetically engineered cell is modified with gene expression modules wherein the expression from any one of the expression modules is constitutive or is tuneable.

Said expression modules are also known as transcriptional units and comprise polynucleotides for expression of recombinant genes including coding gene sequences and appropriate transcriptional and/or translational control signals that are operably linked to the coding genes. The control signals comprise promoter sequences, untranslated regions, ribosome binding sites, terminator sequences. The expression modules can contain elements for expression of one single recombinant gene but can also contain elements for expression of more recombinant genes or can be organized in an operon structure for integrated expression of two or more recombinant genes. The polynucleotides may be produced by recombinant DNA technology using techniques well-known in the art. Methods that are well known to those skilled in the art to construct expression modules include, for example, in vitro recombinant DNA techniques, synthetic techniques, and in vivo genetic recombination. See, for example, the techniques described in Sambrook et al. (2001) Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to a preferred aspect of the disclosure, the cell is modified with one or more expression modules. The expression modules can be integrated in the genome of the cell or can be presented to the cell on a vector. The vector can be present in the form of a plasmid, cosmid, phage, liposome, or virus, which is to be stably transformed/transfected into the metabolically engineered cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., see above. For recombinant production, cells can be genetically engineered to incorporate expression systems or portions thereof or polynucleotides of the disclosure. Introduction of a polynucleotide into the cell can be effected by methods described in many standard laboratory manuals, such as Davis et al., Basic Methods in Molecular Biology, (1986), and Sambrook et al., 1989, supra.

As used herein an expression module comprises polynucleotides for expression of at least one recombinant gene. The recombinant gene is involved in the pathway for production of a negatively charged, preferably sialylated, oligosaccharide; or the recombinant gene is linked to other pathways in the cell that are not involved in the synthesis of a negatively charged, preferably sialylated, oligosaccharide. The recombinant genes encode endogenous proteins with a modified expression or activity, preferably the endogenous proteins are overexpressed; or the recombinant genes encode heterologous proteins that are heterogeneously introduced and expressed in the modified cell, preferably overexpressed. The endogenous proteins can have a modified expression in the cell, which also expresses a heterologous protein.

According to a preferred aspect of the disclosure, the expression of each of the expression modules is constitutive or tuneable as defined herein.

In a preferred embodiment of the method and/or cell of the disclosure, the cell is modified in the expression or activity of a hydrolyzing UDP-N-acetyl-D-glucosamine-2-epimerase as described herein. Preferably, the hydrolyzing UDP-N-acetyl-D-glucosamine-2-epimerase is an endogenous protein of the cell with a modified expression or activity, preferably the endogenous hydrolyzing UDP-N-acetyl-D-glucosamine-2-epimerase is overexpressed; alternatively the hydrolyzing UDP-N-acetyl-D-glucosamine-2-epimerase is a heterologous protein that is heterogeneously introduced and expressed in the cell, preferably overexpressed. The endogenous hydrolyzing UDP-N-acetyl-D-glucosamine-2-epimerase can have a modified expression in the cell, which also expresses a heterologous hydrolyzing UDP-N-acetyl-D-glucosamine-2-epimerase.

In the scope of the disclosure, the hydrolyzing UDP-N-acetyl-D-glucosamine-2-epimerase comprises an amino acid sequence comprising a conserved motif [ACILM][GST]N[ST][ST]XXXX[DE][ACGILMST][ACDEGIMPQSTV] with SEQ ID NO:12, wherein X can be any amino acid residue, and has hydrolyzing UDP-N-acetyl-D-glucosamine-2-epimerase activity catalyzing the reaction UDP-N-acetyl-D-glucosamine (UDP-GlcNAc)=N-acetylmannosamine (ManNAc). The X present in the conserved motif indicates that any single amino acid is possible. Herein, the amino acid can be one of the 20 common amino acids encoded in the genetic code of life (i.e., A, R, N, D, C, Q, E, G, H, I, L, K, M, F, P, S, T, W, Y or V) or can be a modified amino acid (e.g., due to decomposition of a common amino acid like e.g., L-ornithine, or modified by e.g., hydroxylation, carboxylation, phosphorylation, methylation, acetylation, glycosylation, ADP-ribosylation or ubiquitination of a common amino acid). If multiple Xs are present, like in SEQ ID NO: 12, each X is an amino acid that results from a new, independent selection made out of the list of possible amino acid residues. In this way, each X present in the conserved motif with SEQ ID NO: 12 refers to the same amino acid or to a different amino acid.

In a preferred embodiment of the method and/or cell of the disclosure, the hydrolyzing UDP-N-acetyl-D-glucosamine-2-epimerase comprises, consists of or consists essentially of an amino acid sequence that is at least 80% identical over a stretch of at least 150 amino acid residues to any one of the amino acid sequences as represented by SEQ ID NOs:01, 02, 03, 04, 05, 06, 07 or 08. In a more preferred embodiment, the hydrolyzing UDP-N-acetyl-D-glucosamine-2-epimerase comprises, consists of or consists essentially of an amino acid sequence that is at least 80% identical over a stretch of at least 200 amino acid residues to any one of the amino acid sequences as represented by SEQ ID NOs:01, 02, 03, 04, 05, 06, 07 or 08.

In another and/or additional preferred embodiment of the method and/or cell of the disclosure, the hydrolyzing UDP-N-acetyl-D-glucosamine-2-epimerase comprises an amino acid sequence comprising an IPR domain selected from the list comprising IPR003331, IPR020004 and IPR029767 as defined by InterPro 90.0 as released on 4 Aug. 2022.

In another and/or additional preferred embodiment of the method and/or cell of the disclosure, the hydrolyzing UDP-N-acetyl-D-glucosamine-2-epimerase comprises an amino acid sequence comprising a PF02350 motif as defined by PFAM 32.0 as released in September 2018.

In another and/or additional preferred embodiment of the method and/or cell of the disclosure, the hydrolyzing UDP-N-acetyl-D-glucosamine-2-epimerase comprises an amino acid sequence comprising a cd03786 motif as defined by CDD v3.17 as released in September 2016.

In another and/or additional preferred embodiment of the method and/or cell of the disclosure, the hydrolyzing UDP-N-acetyl-D-glucosamine-2-epimerase comprises an amino acid sequence comprising a panther domain selected from the list comprising PTHR43174 and PTHR43174:SF3 as defined by PANTHER 17.0 as released on 23 Feb. 2022.

In another and/or additional preferred embodiment of the method and/or cell of the disclosure, the hydrolyzing UDP-N-acetyl-D-glucosamine-2-epimerase comprises an amino acid sequence that is at least 80%, at least 85%, at least 90%, at least 95%, at least 96%, at least 97%, at least 98%, at least 98.5%, or at least 99% identical to any one of the amino acid sequences as represented by SEQ ID NOs:01, 02, 03, 04, 05, 06, 07 or 08 over a stretch of at least 150, at least 160, at least 170, at least 180, at least 190, at least 200, at least 210, at least 220, at least 230, at least 240, at least 250, at least 260, at least 270, at least 280, at least 290 or at least 300 amino acid residues.

In another and/or additional preferred embodiment of the method and/or cell of the disclosure, the hydrolyzing UDP-N-acetyl-D-glucosamine-2-epimerase comprises an amino acid sequence that is at least 80%, at least 85%, at least 90%, at least 95%, at least 96%, at least 97%, at least 98%, at least 98.5%, or at least 99% identical to any one of the full-length amino acid sequences as represented by SEQ ID NOs:01, 02, 03, 04, 05, 06, 07 or 08.

In another and/or additional preferred embodiment of the method and/or cell of the disclosure, the hydrolyzing UDP-N-acetyl-D-glucosamine-2-epimerase comprises an amino acid sequence as represented by any one of SEQ ID NOs:01, 02, 03, 04, 05, 06, 07 or 08 and comprising UDP-N-acetyl-D-glucosamine-2-epimerase activity.

In another and/or additional preferred embodiment of the method and/or cell of the disclosure, the hydrolyzing UDP-N-acetyl-D-glucosamine-2-epimerase is a nnaA polypeptide.

In a preferred embodiment of the method and/or cell of the disclosure, the cell contains a nucleic acid molecule, which comprises a polynucleotide sequence that encodes a hydrolyzing UDP-N-acetyl-D-glucosamine-2-epimerase as described herein. In a more preferred embodiment, the nucleic acid molecule is operably linked to control sequences recognized by the cell, the nucleic acid molecule further i) being integrated in the genome of the cell and/or ii) presented to the cell on a vector. In an additional and/or alternative more preferred embodiment, the nucleic acid molecule is foreign to the cell.

In a preferred embodiment of the method and/or cell of the disclosure, the cell is genetically engineered for production of a negatively charged, preferably sialylated, oligosaccharide, wherein the cell comprises a pathway for production of the oligosaccharide. In a more preferred embodiment, the cell is genetically engineered for production of two or more negatively charged, preferably sialylated, oligosaccharides. In another and/or additional preferred embodiment, the cell is genetically engineered for an enhanced production of a negatively charged, preferably sialylated, oligosaccharide, an enhanced uptake of one or more precursor(s) and/or acceptor(s) that is/are used in the synthesis of a negatively charged, preferably sialylated, oligosaccharide, a better and/or enhanced efflux of the negatively charged, preferably sialylated, oligosaccharide, a decreased production of by-products like e.g., acids, an increased availability of co-factors like e.g., ATP, NADP, NADPH, and/or better and/or enhanced metabolic flux through any one of the sialylation, fucosylation, galactosylation, N-acetylglucosaminylation, N-acetylgalactosaminylation, mannosylation, and/or N-acetylmannosaminylation pathway present in the cell.

In a preferred embodiment of the method and/or cell of the disclosure, the pathway for production of the negatively charged, preferably sialylated, oligosaccharide is a sialylation pathway. In a more preferred embodiment, the cell is genetically engineered to comprise the sialylation pathway. In an even more preferred embodiment, the cell comprises the sialylation pathway wherein the sialylation pathway has been genetically engineered. In another even more preferred embodiment, the cell has been metabolically engineered to comprise a sialylation pathway wherein any one or more of the genes selected from the list comprising, consisting of or consisting essentially of L-glutamine-D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, N-acetylglucosamine 1-phosphate uridylyltransferase, glucosamine-1-phosphate acetyltransferase, bifunctional N-acetylglucosamine-1-phosphate uridyltransferase/glucosamine-1-phosphate acetyltransferase, sialic acid synthase, N-acetylneuraminate lyase, N-acylneuraminate-9-phosphate synthase, N-acylneuraminate-9-phosphatase, d-arabinose 5-phosphate isomerase, KDO-8P synthase, KDO 8-phosphate phosphatase, CMP-KDO synthetase, sialic acid transporter, CMP kinase, CMP-sialic acid synthase and sialyltransferase has/have a modified and/or enhanced expression.

In another and/or additional preferred embodiment of the method and/or cell, the cell further comprises a pathway selected from the list comprising, consisting of or consisting essentially of fucosylation pathway, galactosylation pathway, N-acetylglucosaminylation pathway, N-acetylgalactosaminylation pathway, mannosylation pathway and N-acetylmannosaminylation pathway. In a more preferred embodiment, the cell is genetically engineered to comprise at least one pathway selected from the list comprising, consisting of or consisting essentially of the fucosylation pathway, galactosylation pathway, N-acetylglucosaminylation pathway, N-acetylgalactosaminylation pathway, mannosylation pathway and N-acetylmannosaminylation pathway. In an even more preferred embodiment, the cell comprises at least one pathway selected from the list comprising, consisting of or consisting essentially of the fucosylation pathway, galactosylation pathway, N-acetylglucosaminylation pathway, N-acetylgalactosaminylation pathway, mannosylation pathway and N-acetylmannosaminylation pathway wherein at least one of the fucosylation pathway, galactosylation pathway, N-acetylglucosaminylation pathway, N-acetylgalactosaminylation pathway, mannosylation pathway and N-acetylmannosaminylation pathway has/have been genetically engineered.

In another and/or additional preferred embodiment of the method and/or cell, the cell comprises a fucosylation pathway. In a more preferred additional and/or alternative embodiment, the cell is metabolically engineered to comprise a fucosylation pathway. In another even more preferred additional and/or alternative embodiment, the cell has been metabolically engineered to comprise a fucosylation pathway wherein any one or more of the genes selected from the list comprising, consisting of or consisting essentially of mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase and fucosyltransferase has/have a modified and/or enhanced expression.

In another and/or additional preferred embodiment of the method and/or cell, the cell comprises a galactosylation pathway. In a more preferred additional and/or alternative embodiment, the cell is metabolically engineered to comprise a galactosylation pathway. In another even more preferred additional and/or alternative embodiment, the cell has been metabolically engineered to comprise a galactosylation pathway wherein any one or more of the genes selected from the list comprising, consisting of or consisting essentially of galactose-1-epimerase, galactokinase, glucokinase, galactose-1-phosphate uridylyltransferase, UDP-glucose 4-epimerase, glucose-1-phosphate uridylyltransferase, phosphoglucomutase and galactosyltransferase has/have a modified and/or enhanced expression.

In another and/or additional preferred embodiment of the method and/or cell, the cell comprises an ‘N-acetylglucosaminylation’ pathway. In a more preferred additional and/or alternative embodiment, the cell is metabolically engineered to comprise an N-acetylglucosaminylation pathway. In another even more preferred additional and/or alternative embodiment, the cell has been metabolically engineered to comprise an N-acetylglucosaminylation pathway wherein any one or more of the genes selected from the list comprising, consisting of or consisting essentially of L-glutamine-D-fructose-6-phosphate aminotransferase, N-acetylglucosamine-6-phosphate deacetylase, phosphoglucosamine mutase, N-acetylglucosamine-1-phosphate uridylyltransferase, glucosamine-1-phosphate acetyltransferase, bifunctional N-acetylglucosamine-1-phosphate uridyltransferase/glucosamine-1-phosphate acetyltransferase, and a glycosyltransferase transferring GlcNAc has/have a modified and/or enhanced expression.

In another and/or additional preferred embodiment of the method and/or cell, the cell comprises an ‘N-acetylgalactosaminylation’ pathway. In a more preferred additional and/or alternative embodiment, the cell is metabolically engineered to comprise an N-acetylgalactosaminylation pathway. In another even more preferred additional and/or alternative embodiment, the cell has been metabolically engineered to comprise an N-acetylgalactosaminylation pathway wherein any one or more of the genes selected from the list comprising, consisting of or consisting essentially of L-glutamine-D-fructose-6-phosphate aminotransferase, phosphoglucosamine mutase, N-acetylglucosamine 1-phosphate uridylyltransferase, glucosamine-1-phosphate acetyltransferase, bifunctional N-acetylglucosamine-1-phosphate uridyltransferase/glucosamine-1-phosphate acetyltransferase, UDP-N-acetylglucosamine 4-epimerase, UDP-glucose 4-epimerase, N-acetylgalactosamine kinase and/or UDP-N-acetylgalactosamine pyrophosphorylase and a glycosyltransferase transferring GalNAc has/have a modified and/or enhanced expression.

In another and/or additional preferred embodiment of the method and/or cell, the cell comprises a ‘mannosylation’ pathway. In a more preferred additional and/or alternative embodiment, the cell is metabolically engineered to comprise a mannosylation pathway. In another even more preferred additional and/or alternative embodiment, the cell has been metabolically engineered to comprise a mannosylation pathway wherein any one or more of the genes selected from the list comprising, consisting of or consisting essentially of mannose-6-phosphate isomerase, phosphomannomutase and/or mannose-1-phosphate guanylyltransferase and mannosyltransferase has/have a modified and/or enhanced expression.

In another and/or additional preferred embodiment of the method and/or cell, the cell comprises an ‘N-acetylmannosaminylation’ pathway. In a more preferred additional and/or alternative embodiment, the cell is metabolically engineered to comprise an N-acetylmannosaminylation pathway. In another even more preferred additional and/or alternative embodiment, the cell has been metabolically engineered to comprise an N-acetylmannosaminylation pathway wherein any one or more of the genes selected from the list comprising, consisting of or consisting essentially of L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, bifunctional N-acetylglucosamine-1-phosphate uridyltransferase/glucosamine-1-phosphate acetyltransferase, UDP-GlcNAc 2-epimerase and/or ManNAc kinase and a glycosyltransferase transferring ManNAc has/have a modified and/or enhanced expression.

In another and/or additional preferred embodiment of the method and/or cell of the disclosure, the cell comprises one or more pathway(s) for monosaccharide synthesis. The pathways for monosaccharide synthesis comprise enzymes like e.g., carboxylases, decarboxylases, isomerases, epimerases, reductases, enolases, phosphorylases, carboxykinases, kinases, phosphatases, aldolases, hydrolases, dehydrogenases, enzymes involved in the synthesis of one or more nucleoside triphosphate(s) like UTP, GTP, ATP and CTP, enzymes involved in the synthesis of any one or more nucleoside mono- or diphosphates like e.g., UMP and UDP, respectively, and enzymes involved in the synthesis of phosphoenolpyruvate (PEP).

In another and/or additional preferred embodiment of the method and/or cell of the disclosure, the cell comprises one or more pathway(s) for phosphorylated monosaccharide synthesis. The pathways for phosphorylated monosaccharide synthesis comprise enzymes involved in the synthesis of one or more monosaccharide(s), one or more nucleoside mono-, di- and/or triphosphate(s) and enzymes involved in the synthesis of phosphoenolpyruvate (PEP) like e.g., but not limited to PEP synthase, carboxylases, decarboxylases, isomerases, epimerases, reductases, enolases, phosphorylases, carboxykinases, kinases, phosphatases, aldolases, hydrolases and dehydrogenases. In another and/or additional preferred embodiment of the method and/or cell of the disclosure, the cell comprises one or more pathways for the synthesis of one or more nucleotide-activated sugars. The pathways for nucleotide-activated sugar synthesis comprise enzymes like e.g., PEP synthase, carboxylases, decarboxylases, isomerases, epimerases, reductases, enolases, phosphorylases, carboxykinases, kinases, phosphatases, aldolases, hydrolases, dehydrogenases, mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridyltransferase, glucosamine-1-phosphate acetyltransferase, bifunctional N-acetylglucosamine-1-phosphate uridyltransferase/glucosamine-1-phosphate acetyltransferase, sialic acid synthase, N-acetylneuraminate lyase, N-acylneuraminate-9-phosphate synthase, N-acylneuraminate-9-phosphate phosphatase, d-arabinose 5-phosphate isomerase, KDO-8P synthase, KDO 8-phosphate phosphatase, CMP-KDO synthetase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase, glucophosphomutase and/or N-acetylglucosamine-1-phosphate uridylyltransferase.

In another and/or additional preferred embodiment of the method and/or cell, the cell possesses, preferably expresses, more preferably overexpresses, one or more glycosyltransferase(s) selected from the list comprising, consisting of or consisting essentially of fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

In a more preferred embodiment of the method and/or cell of the disclosure, the fucosyltransferase is selected from the list comprising, consisting of or consisting essentially of alpha-1,2-fucosyltransferase, alpha-1,3-fucosyltransferase, alpha-1,4-fucosyltransferase and alpha-1,6-fucosyltransferase.

In an alternative and/or additional more preferred embodiment of the method and/or cell of the disclosure, the further sialyltransferase is selected from the list comprising, consisting of or consisting essentially of alpha-2,3-sialyltransferase, alpha-2,6-sialyltransferase, and alpha-2,8-sialyltransferase.

In an alternative and/or additional more preferred embodiment of the method and/or cell of the disclosure, the galactosyltransferase is selected from the list comprising, consisting of or consisting essentially of beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase.

In an alternative and/or additional more preferred embodiment of the method and/or cell of the disclosure, the glucosyltransferase is selected from the list comprising, consisting of or consisting essentially of alpha-glucosyltransferase, beta-1,2-glucosyltransferase, beta-1,3-glucosyltransferase and beta-1,4-glucosyltransferase.

In an alternative and/or additional more preferred embodiment of the method and/or cell of the disclosure, the mannosyltransferase is selected from the list comprising, consisting of or consisting essentially of alpha-1,2-mannosyltransferase, alpha-1,3-mannosyltransferase and alpha-1,6-mannosyltransferase.

In an alternative and/or additional more preferred embodiment of the method and/or cell of the disclosure, the N-acetylglucosaminyltransferase is selected from the list comprising, consisting of or consisting essentially of galactoside beta-1,3-N-acetylglucosaminyltransferase and beta-1,6-N-acetylglucosaminyltransferase.

In an alternative and/or additional more preferred embodiment of the method and/or cell of the disclosure, the N-acetylgalactosaminyltransferase is selected from the list comprising, consisting of or consisting essentially of alpha-1,3-N-acetylgalactosaminyltransferase.

In an alternative and/or additional more preferred embodiment of the method and/or cell of the disclosure, the cell is modified in the expression or activity of at least one of the glycosyltransferases. In a preferred embodiment, the glycosyltransferase is an endogenous protein of the cell with a modified expression or activity, preferably the endogenous glycosyltransferase is overexpressed; alternatively the glycosyltransferase is a heterologous protein that is heterogeneously introduced and expressed in the cell, preferably overexpressed. The endogenous glycosyltransferase can have a modified expression in the cell, which also expresses a heterologous glycosyltransferase.

In another and/or additional preferred embodiment of the method and/or cell, the cell is capable to produce, preferably produces, one or more nucleotide-activated sugars, preferably the cell is genetically engineered for production of one or more of the nucleotide-activated sugar(s). Herein, the one or more nucleotide-activated sugar(s) is/are selected from the list comprising, consisting of or consisting essentially of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid, CMP-Neu5Ac, CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), CMP-2-keto-3-deoxymanno-octulonic acid (CMP-KDO), GDP-rhamnose and UDP-xylose.

In another and/or additional preferred embodiment of the method and/or cell, the cell comprises a pathway for the synthesis of a nucleotide-activated sugar selected from the list comprising, consisting of or consisting essentially of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid, CMP-Neu5Ac, CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), CMP-2-keto-3-deoxymanno-octulonic acid (CMP-KDO), GDP-rhamnose and UDP-xylose.

The cell used herein is optionally genetically modified to express the de novo synthesis of UDP-GlcNAc. UDP-GlcNAc can be provided by an enzyme expressed in the cell or by the metabolism of the cell. Such cell producing an UDP-GlcNAc can express enzymes converting, e.g., GlcNAc, which is to be added to the cell, to UDP-GlcNAc. These enzymes may be any one or more of the list comprising, consisting of or consisting essentially of an N-acetyl-D-glucosamine kinase, an N-acetylglucosamine-6-phosphate deacetylase, a phosphoglucosamine mutase, N-acetylglucosamine-1-phosphate uridyltransferase, glucosamine-1-phosphate acetyltransferase, and a bifunctional N-acetylglucosamine-1-phosphate uridyltransferase/glucosamine-1-phosphate acetyltransferase from several species including Homo sapiens, Escherichia coli. Preferably, the cell is modified to produce UDP-GlcNAc.

Additionally, or alternatively, the cell used herein is optionally genetically modified to express the de novo synthesis of CMP-Neu5Ac. CMP-Neu5Ac can be provided by an enzyme expressed in the cell or by the metabolism of the cell. Such cell producing CMP-Neu5Ac can express an enzyme converting, e.g., sialic acid to CMP-Neu5Ac. This enzyme may be a CMP-sialic acid synthetase, like the N-acylneuraminate cytidylyltransferase from several species including Homo sapiens, Neisseria meningitidis, and Pasteurella multocida. Preferably, the cell is modified to produce CMP-Neu5Ac. More preferably, the cell is modified for enhanced CMP-Neu5Ac production. The modification can be any one or more selected from the list comprising, consisting of or consisting essentially of knock-out of an N-acetylglucosamine-6-phosphate deacetylase, knock-out of a glucosamine-6-phosphate deaminase, over-expression of a CMP-sialic acid synthetase, and over-expression of an N-acetyl-D-glucosamine-2-epimerase encoding gene.

Additionally, or alternatively, the cell used herein is optionally genetically modified to express the de novo synthesis of GDP-fucose. GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. The modification can be any one or more selected from the list comprising, consisting of or consisting essentially of knock-out of an UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene.

Additionally, or alternatively, the cell used herein is optionally genetically modified to express the de novo synthesis of UDP-Gal. UDP-Gal can be provided by an enzyme expressed in the cell or by the metabolism of the cell. Such cell producing UDP-Gal can express an enzyme converting, e.g., UDP-glucose, to UDP-Gal. This enzyme may be, e.g., the UDP-glucose-4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. The modification can be any one or more selected from the list comprising, consisting of or consisting essentially of knock-out of a bifunctional 5′-nucleotidase/UDP-sugar hydrolase encoding gene, knock-out of a galactose-1-phosphate uridylyltransferase encoding gene and over-expression of an UDP-glucose-4-epimerase encoding gene.

Additionally, or alternatively, the cell used herein is optionally genetically modified to express the de novo synthesis of UDP-GalNAc. UDP-GalNAc can be synthesized from UDP-GlcNAc by the action of a single-step reaction using an UDP-N-acetylglucosamine 4-epimerase like e.g., wbgU from Plesiomonas shigelloides, gne from Yersinia enterocolitica or wbpP from Pseudomonas aeruginosa serotype O6. Preferably, the cell is modified to produce UDP-GalNAc. More preferably, the cell is modified for enhanced UDP-GalNAc production.

Additionally, or alternatively, the cell used herein is optionally genetically modified to express the de novo synthesis of UDP-ManNAc. UDP-ManNAc can be synthesized directly from UDP-GlcNAc via an epimerization reaction performed by an UDP-GlcNAc 2-epimerase (like e.g., cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production.

In another and/or additional preferred embodiment of the method and/or cell, the cell possesses, preferably expresses, one or more genes selected from the list comprising, consisting of or consisting essentially of mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-glutamine-D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, N-acetylglucosamine 1-phosphate uridylyltransferase, glucosamine-1-phosphate acetyltransferase, bifunctional N-acetylglucosamine-1-phosphate uridyltransferase/glucosamine-1-phosphate acetyltransferase, sialic acid synthase, N-acetylneuraminate lyase, N-acylneuraminate-9-phosphate synthase, N-acylneuraminate-9-phosphatase, d-arabinose 5-phosphate isomerase, KDO-8P synthase, KDO 8-phosphate phosphatase, CMP-KDO synthetase, sialic acid transporter, CMP kinase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase. In a more preferred embodiment, the cell overexpresses one or more genes selected from the list comprising, consisting of or consisting essentially of mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-glutamine-D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, sialic acid synthase, N-acetylneuraminate lyase, N-acylneuraminate-9-phosphate synthase, N-acylneuraminate-9-phosphatase, d-arabinose 5-phosphate isomerase, KDO-8P synthase, KDO 8-phosphate phosphatase, CMP-KDO synthetase, sialic acid transporter, CMP kinase, CMP-sialic acid synthase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase.

In another and/or additional preferred embodiment of the method and/or cell, the cell is capable to produce, preferably produces, N-acetylmannosamine (ManNAc), a sialic acid residue and/or N-acetylglucosamine (GlcNAc), wherein the sialic acid residue is selected from the list comprising Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4; Neu4,5,7,8,9Ac5; Neu5Gc and 2-keto-3-deoxymanno-octulonic acid (KDO). In a more preferred embodiment, the cell comprises a pathway for production of ManNAc, a sialic acid residue and/or GlcNAc. In another and/or additional more preferred embodiment, the cell is genetically engineered for production of ManNAc, a sialic acid residue and/or GlcNAc.

In another and/or additional preferred embodiment of the method and/or cell, the negatively charged oligosaccharide produced by a cell of the disclosure is a sialylated oligosaccharide having at least one sialic acid residue selected from the list comprising, consisting of or consisting essentially of Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4; Neu4,5,7,8,9Ac5; Neu5Gc and 2-keto-3-deoxymanno-octulonic acid (KDO).

In a more preferred embodiment, the negatively charged oligosaccharide is an oligosaccharide selected from the list comprising, consisting of or consisting essentially of a negatively charged, preferably sialylated, milk oligosaccharide, preferably a negatively charged, more preferably sialylated, mammalian milk oligosaccharide (MMO), more preferably a negatively charged, more preferably sialylated, human milk oligosaccharide (HMO); O-antigen; the oligosaccharide repeats present in capsular polysaccharides; peptidoglycan; an amino-sugar; Lewis-type antigen oligosaccharide; a negatively charged, preferably sialylated, animal oligosaccharide; a negatively charged, preferably sialylated, plant oligosaccharide; N-acetyllactosamine containing negatively charged, preferably sialylated, oligosaccharide and lacto-N-biose containing negatively charged, preferably sialylated, oligosaccharide. In an even more preferred embodiment, the negatively charged oligosaccharide is a mammalian milk oligosaccharide (MMO) as described herein. In another more preferred embodiment, the negatively charged oligosaccharide is a human milk oligosaccharide (HMO) as described herein. In another more preferred embodiment, the negatively charged oligosaccharide is an animal oligosaccharide selected from the list consisting of N-glycans and O-glycans. In another more preferred embodiment, the negatively charged oligosaccharide is a plant oligosaccharide selected from the list consisting of N-glycans and O-glycans. In the context of the disclosure, N-glycans and O-glycans refer to the oligosaccharide structures as known by the person skilled in the art wherein the structures are not attached to a protein or a peptide.

In an even more preferred embodiment, the negatively charged oligosaccharide is selected from the list comprising 3′sialyllactose (3′SL), 6′sialyllactose (6′SL), 3′-sialyllactosamine, 3,6-disialyllactose, 8,3-disialyllactose, sialylated lacto-N-triose, sialyllacto-N-tetraose a (LSTa), KDOα-2,3Galβ-1,3GlcNAcβ-1,3Galβ-1,4Glc, sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose, Neu5Ac-a2,3-Gal-b1,4-GlcNAc-b1,3-Gal, Neu5Ac-a2,3-Gal-b1,3-GlcNAc-b1,3-Gal, 3′-KDO-lactose, 3′-KDO-lactosamine, 3′-KDO-lacto-N-biose, 3′-KDO-6′sialyllactose, 3′KDO-8-sialyllactose, KDO-2,3Galβ-1,3GalNaco-1,3Galα-1,4Galβ-1,4Gal, KDOα-2,3Galβ-1,4GlcNacβ-1,3Galβ-1,4Glc, 3′-KDO-3-fucosyllactose, Neu5Ac-a2,8-Neu5Ac-a2,3-Gal-b1,3-GlcNAc-b1,3-Gal, 3′-Sialyl-2′-fucosyllactose, 6′-Sialyl-2′-fucosyllactose, 6′-Sialyl-3-fucosyllactose, Neu5Ac-a2,6-(Neu5Ac-a2,3-)Gal-b1,4-Glc, 3′-Sialyl-3-fucosyllactosamine, Fuc-a1,4-(Neu5Ac-a2,3-Gal-b1,3-)GlcNAc, 6′-Sialyllacto-N-biose, 3′-Sialyllacto-N-biose, Neu5Ac-a2,6-(GlcNAc-b1,3-)Gal-b1,4-Glc, Neu5Ac-a2,6-(Gal-b1,4-(Fuc-a1,3-)GlcNAc-b1,3-)Gal-b1,4-Glc, Neu5Ac-a2,3-Gal-b1,4-(Fuc-a1,3-)GlcNAc-b1,3-Gal-b1,4-Glc, Neu5Ac-a2,6-(Neu5Ac-a2,6-Gal-b1,4-(Fuc-a1,3-)GlcNAc-b1,3-)Gal-b1,4-Glc, Neu5Ac-a2,6-(Gal-b1,4-GlcNAc-b1,3-)Gal-b1,4-Glc, Neu5Ac-a2,6-(Gal-b1,3-GlcNAc-b1,3-)Gal-b1,4-(Fuc-a1,3-)Glc, Neu5Ac-a2,6-Gal-b1,3-GlcNAc-b1,3-Gal-b1,4-(Fuc-a1,3-)Glc, Neu5Ac-a2,3-Gal-b1,3-GlcNAc-b1,3-Gal-b1,4-(Fuc-a1,3-)Glc, Neu5Ac-a2,3-(Fuc-a1,2-)Gal-b1,3-GlcNAc-b1,3-Gal-b1,4-Glc, Neu5Ac-a2,6-(Fuc-a1,2-Gal-b1,3-GlcNAc-b1,3-)Gal-b1,4-Glc, Neu5Ac-a2,6-(Fuc-a1,2-)Gal-b1,3-GlcNAc-b1,3-Gal-b1,4-Glc, Fuc-a1,4-(Neu5Ac-a2,3-Gal-b1,3-)GlcNAc-b1,3-Gal-b1,4-Glc, Neu5Ac-a2,6-(Neu5Ac-a2,6-Gal-b1,3-GlcNAc-b1,3-)Gal-b1,4-(Fuc-a1,3-)Glc, Neu5Ac-a2,6-(Neu5Ac-a2,6-(Fuc-a1,2-)Gal-b1,3-GlcNAc-b1,3-)Gal-b1,4-Glc, Neu5Ac-a2,6-(Gal-b1,3-GlcNAc-b1,3-)Gal-b1,4-Glc, Neu5Ac-a2,6-Gal-b1,3-GlcNAc-b1,3-Gal-b1,4-Glc, Neu5Ac-a2,6-(Neu5Ac-a2,3)-Gal-b1,4-GlcNAc-b1,3-Gal-b1,4-Glc, Neu5Ac-a2,6-(Neu5Ac-a2,3-Gal-b1,3-GlcNAc-b1,3-)Gal-b1,4-Glc, Neu5Ac-a2,6-(Neu5Ac-a2,6-Gal-b1,3-GlcNAc-b1,3)-Gal-b1,4-Glc, Neu5Ac-a2,6-(Neu5Ac-a2,6-(Neu5Ac-a2,3)-Gal-b1,3-GlcNAc-b1,3)-Gal-b1,4-Glc, Neu5Ac-a2,6-(Neu5Ac-a2,3-Gal-b1,4-GlcNAc-b1,3)-Gal-b1,4-Glc, Neu5Ac-a2,6-(Neu5Ac-a2,6-Gal-b1,4-GlcNAc-b1,3)-Gal-b1,4-Glc, Neu5Ac-a2,6-(Neu5Ac-a2,6-(Neu5Ac-a2,3)-Gal-b1,4-GlcNAc-b1,3)-Gal-b1,4-Glc, Neu5Ac-a2,6-(Gal-b1,4-GlcNAc-b1,3-Gal-b1,4-GlcNAc-b1,3)-Gal-b1,4-Glc, Neu5Ac-a2,6-(Gal-b1,4-GlcNAc-b1,3)-Gal-b1,4-GlcNAc-b1,3-Gal-b1,4-Glc, Neu5Ac-a2,6-Gal-b1,4-GlcNAc-b1,3-Gal-b1,4-GlcNAc-b1,3-Gal-b1,4-Glc, Neu5Ac-a2,6-(Neu5Ac-a2,6-(Neu5Ac-a2,6-Gal-b1,4-GlcNAc-b1,3)-Gal-b1,4-GlcNAc-b1,3)-Gal-b1,4-Glc, Neu5Ac-a2,3-Gal-b1,4-GlcNAc-b1,3-Gal-b1,4-GlcNAc-b1,3-Gal-b1,4-Glc, 6′-KDO-lactose, 6′-KDO-lactosamine, 6′-KDO-lacto-N-biose, KDO-lacto-N-triose, KDO-lacto-N-tetraose, KDO-lacto-N-tetraose, and combinations thereof.

The oligosaccharide in the context of the disclosure is preferably in free form, i.e., the oligosaccharide does not contain any protective group.

In another and/or additional preferred embodiment of the method and/or cell of the disclosure, the cell is capable to produce, preferably produces, the negatively charged, preferably sialylated, oligosaccharide from one or more precursor(s) as defined herein. In a more preferred embodiment, the precursor is lactose. Preferably, the one or more precursor(s) is/are fed to the cell from the culture medium or the incubation. In another more preferred embodiment, the cell is capable to produce, preferably produces, at least one of the one or more precursor(s). In an even more preferred embodiment, the cell is capable to produce, preferably produces, all of the one or more precursor(s). In another more preferred embodiment, the cell is capable to produce, preferably produces, lactose. In another more preferred embodiment, the cell is genetically engineered for the production of at least one of the one or more precursor(s). In an even more preferred embodiment, the cell is genetically engineered for the production of all of the one or more precursor(s). In another more preferred embodiment, the cell is genetically engineered for the production of lactose. In another more preferred embodiment, at least one of the one or more precursor(s) is internalized in the cell via one or more membrane protein(s), preferably the membrane protein(s) is/are membrane transporter protein(s) as described herein. In another preferred embodiment, the precursor(s) that is/are used by the cell for the production of the negatively charged, preferably sialylated, oligosaccharide is/are completely converted into the negatively charged, preferably sialylated, oligosaccharide.

In another and/or additional preferred embodiment of the method and/or cell of the disclosure, the cell is capable to produce, preferably produces, phosphoenolpyruvate (PEP). Preferably, the cell comprises a pathway for production of PEP.

In another and/or additional preferred embodiment of the method and/or cell of the disclosure, the cell is modified for enhanced synthesis and/or supply of phosphoenolpyruvate (PEP) compared to a non-modified progenitor.

In a preferred embodiment and as a means for enhanced production and/or supply of PEP, one or more PEP-dependent, sugar-transporting phosphotransferase system(s) is/are disrupted such as but not limited to: 1) the N-acetyl-D-glucosamine Npi-phosphotransferase (EC 2.7.1.193), which is, for instance, encoded by the nagE gene (or the cluster nagABCD) in E. coli or Bacillus species, 2) ManXYZ, which encodes the Enzyme 11 Man complex (mannose PTS permease, protein-Npi-phosphohistidine-D-mannose phosphotransferase) that imports exogenous hexoses (mannose, glucose, glucosamine, fructose, 2-deoxyglucose, mannosamine, N-acetylglucosamine, etc.) and releases the phosphate esters into the cell cytoplasm, 3) the glucose-specific PTS transporter (for instance, encoded by PtsG/Crr) which takes up glucose and forms glucose-6-phosphate in the cytoplasm, 4) the sucrose-specific PTS transporter, which takes up sucrose and forms sucrose-6-phosphate in the cytoplasm, 5) the fructose-specific PTS transporter (for instance, encoded by the genes fruA and fruB and the kinase fruK, which takes up fructose and forms in a first step fructose-1-phosphate and in a second step fructose1,6 bisphosphate, 6) the lactose PTS transporter (for instance, encoded by lacE in Lactococcus casei) which takes up lactose and forms lactose-6-phosphate, 7) the galactitol-specific PTS enzyme, which takes up galactitol and/or sorbitol and forms galactitol-1-phosphate or sorbitol-6-phosphate, respectively, 8) the mannitol-specific PTS enzyme, which takes up mannitol and/or sorbitol and forms mannitol-1-phosphate or sorbitol-6-phosphate, respectively, and 9) the trehalose-specific PTS enzyme, which takes up trehalose and forms trehalose-6-phosphate.

In another and/or additional preferred embodiment and as a means for enhanced production and/or supply of PEP, the full PTS system is disrupted by disrupting the PtsIH/Crr gene cluster.

In another and/or additional preferred embodiment, the cell is further modified to compensate for the deletion of a PTS system of a carbon source by the introduction and/or overexpression of the corresponding permease. These are e.g., permeases or ABC transporters that comprise but are not limited to transporters that specifically import lactose such as e.g., the transporter encoded by the LacY gene from E. coli, sucrose such as e.g., the transporter encoded by the cscB gene from E. coli, glucose such as e.g., the transporter encoded by the galP gene from E. coli, fructose such as e.g., the transporter encoded by the fruI gene from Streptococcus mutans, or the Sorbitol/mannitol ABC transporter such as the transporter encoded by the cluster SmoEFGK of Rhodobacter sphaeroides, the trehalose/sucrose/maltose transporter such as the transporter encoded by the gene cluster ThuEFGK of Sinorhizobium mehlioti and the N-acetylglucosamine/galactose/glucose transporter such as the transporter encoded by NagP of Shewanella oneidensis. Examples of combinations of PTS deletions with overexpression of alternative transporters are: 1) the deletion of the glucose PTS system, e.g., ptsG gene, combined with the introduction and/or overexpression of a glucose permease (e.g., galP of glcP), 2) the deletion of the fructose PTS system, e.g., one or more of the fruB, fruA, fruK genes, combined with the introduction and/or overexpression of fructose permease, e.g., fruI, 3) the deletion of the lactose PTS system, combined with the introduction and/or overexpression of lactose permease, e.g., LacY, and/or 4) the deletion of the sucrose PTS system, combined with the introduction and/or overexpression of a sucrose permease, e.g., cscB.

In a further preferred embodiment, the cell is modified to compensate for the deletion of a PTS system of a carbon source by the introduction of carbohydrate kinases, such as glucokinase (EC 2.7.1.1, EC 2.7.1.2, EC 2.7.1.63), galactokinase (EC 2.7.1.6), and/or fructokinase (EC 2.7.1.3, EC 2.7.1.4).

In another and/or additional preferred embodiment and as a means for enhanced production and/or supply of PEP, the cell is modified by the introduction of or modification in any one or more of the list comprising phosphoenolpyruvate synthase activity (EC: 2.7.9.2 encoded, for instance, in E. coli by ppsA), phosphoenolpyruvate carboxykinase activity (EC 4.1.1.32 or EC 4.1.1.49 encoded, for instance, in Corynebacterium glutamicum by PCK or in E. coli by pckA, resp.), phosphoenolpyruvate carboxylase activity (EC 4.1.1.31 encoded, for instance, in E. coli by ppc), oxaloacetate decarboxylase activity (EC 4.1.1.112 encoded, for instance, in E. coli by eda), pyruvate kinase activity (EC 2.7.1.40 encoded, for instance, in E. coli by pykA and pykF), pyruvate carboxylase activity (EC 6.4.1.1 encoded, for instance, in B. subtilis by pyc) and malate dehydrogenase activity (EC 1.1.1.38 or EC 1.1.1.40 encoded, for instance, in E. coli by maeA or maeB, resp.).

In another and/or additional preferred embodiment and as a means for enhanced production and/or supply of PEP, the cell is modified by a reduced activity of phosphoenolpyruvate carboxylase activity, and/or pyruvate kinase activity, preferably a deletion of at least one of the genes encoding for phosphoenolpyruvate carboxylase, the pyruvate carboxylase activity and/or pyruvate kinase.

In another and/or additional preferred embodiment of the method and/or cell of the disclosure, the cell is further modified for reduced degradation of acetyl-CoA and/or its main precursor pyruvate.

In another and/or additional preferred embodiment of the method and/or cell of the disclosure, the cell comprises a modification for reduced production of acetate compared to a non-modified progenitor. The modification can be any one or more selected from the list comprising, consisting of or consisting essentially of overexpression of an acetyl-coenzyme A synthetase, a full or partial knock-out or rendered less functional pyruvate dehydrogenase and a full or partial knock-out or rendered less functional lactate dehydrogenase.

In another and/or additional preferred embodiment of the method and/or cell of the disclosure, the cell is modified in the expression or activity of at least one acetyl-coenzyme A synthetase like e.g., acs from E. coli, S. cerevisiae, H. sapiens, M musculus. In a preferred embodiment, the acetyl-coenzyme A synthetase is an endogenous protein of the cell with a modified expression or activity, preferably the endogenous acetyl-coenzyme A synthetase is overexpressed; alternatively, the acetyl-coenzyme A synthetase is a heterologous protein that is heterogeneously introduced and expressed in the cell, preferably overexpressed. The endogenous acetyl-coenzyme A synthetase can have a modified expression in the cell, which also expresses a heterologous acetyl-coenzyme A synthetase.

In an alternative and/or additional further preferred embodiment of the method and/or cell of the disclosure, the cell is modified in the expression or activity of at least one pyruvate dehydrogenase like e.g., from E. coli, S. cerevisiae, H. sapiens and R. norvegicus. In a preferred embodiment, the cell has been modified to have at least one partially or fully knocked out or mutated pyruvate dehydrogenase encoding gene by means generally known by the person skilled in the art resulting in at least one protein with less functional or being disabled for pyruvate dehydrogenase activity. In a more preferred embodiment, the cell has a full knock-out in the poxB encoding gene resulting in a cell lacking pyruvate dehydrogenase activity.

In an alternative and/or additional further preferred embodiment of the method and/or cell of the disclosure, the cell is modified in the expression or activity of at least one lactate dehydrogenase like e.g., from E. coli, S. cerevisiae, H. sapiens and R. norvegicus. In a preferred embodiment, the cell has been modified to have at least one partially or fully knocked out or mutated lactate dehydrogenase encoding gene by means generally known by the person skilled in the art resulting in at least one protein with less functional or being disabled for lactate dehydrogenase activity. In a more preferred embodiment, the cell has a full knock-out in the ldhA encoding gene resulting in a cell lacking lactate dehydrogenase activity.

According to another and/or additional preferred embodiment of the method and/or cell of the disclosure, the cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose:undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, lon protease, glucose-specific translocating phosphotransferase enzyme IIBC component ptsG, glucose-specific translocating phosphotransferase (PTS) enzyme IIBC component malX, enzyme IIA^{Glc}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

In another and/or additional preferred embodiment of the method and/or cell of the disclosure, the cell comprises a catabolic pathway for selected mono-, di- or oligosaccharides, which is at least partially inactivated, the mono-, di-, or oligosaccharides being involved in and/or required for the synthesis of the negatively charged, preferably sialylated, oligosaccharide of the disclosure.

In another and/or additional preferred embodiment of the method and/or cell of the disclosure, the cell possesses, expresses and/or overexpresses at least one gene selected from the list comprising, consisting of or consisting essentially of genes encoding a disulfide bond isomerase, a thiol oxidase or a chaperone.

In another and/or additional preferred embodiment of the method and/or cell of the disclosure, the cell expresses a membrane transporter protein or a polypeptide having transport activity hereby transporting compounds across the outer membrane of the cell wall. In another and/or additional preferred embodiment of the method and/or cell of the disclosure, the cell expresses more than one membrane transporter protein or polypeptide having transport activity hereby transporting compounds across the outer membrane of the cell wall. In a more preferred embodiment, the cell is modified in the expression or activity of the membrane transporter protein or polypeptide having transport activity. The membrane transporter protein or polypeptide having transport activity is an endogenous protein of the cell with a modified expression or activity, preferably the endogenous membrane transporter protein or polypeptide having transport activity is overexpressed; alternatively, the membrane transporter protein or polypeptide having transport activity is a heterologous protein that is heterogeneously introduced and expressed in the cell, preferably overexpressed. The endogenous membrane transporter protein or polypeptide having transport activity can have a modified expression in the cell, which also expresses a heterologous membrane transporter protein or polypeptide having transport activity.

In another and/or additional more preferred embodiment, the membrane transporter protein or polypeptide having transport activity is selected from the list comprising, consisting of or consisting essentially of porters, P—P-bond-hydrolysis-driven transporters, β-barrel porins, auxiliary transport proteins and phosphotransfer-driven group translocators. In an even more preferred embodiment, the porters comprise MFS transporters, sugar efflux transporters (SET) and siderophore exporters. In another more preferred embodiment, the P—P-bond-hydrolysis-driven transporters comprise ABC transporters and siderophore exporters.

In another and/or additional preferred embodiment of the method and/or cell of the disclosure, the membrane transporter protein or polypeptide having transport activity controls the flow over the outer membrane of the cell wall of the negatively charged, preferably sialylated, oligosaccharide. In an alternative and/or additional preferred embodiment of the method and/or cell of the disclosure, the membrane transporter protein or polypeptide having transport activity controls the flow over the outer membrane of the cell wall of one or more precursor(s) to be used in the production of the negatively charged, preferably sialylated, oligosaccharide.

In another preferred embodiment of the method and/or cell of the disclosure, the membrane transporter protein or polypeptide having transport activity provides improved production of the negatively charged, preferably sialylated, oligosaccharide. In an alternative and/or additional preferred embodiment of the method and/or cell of the disclosure, the membrane transporter protein or polypeptide having transport activity provides enabled efflux of the negatively charged, preferably sialylated, oligosaccharide. In an alternative and/or additional preferred embodiment of the method and/or cell of the disclosure, the membrane transporter protein or polypeptide having transport activity provides enhanced efflux of the negatively charged, preferably sialylated, oligosaccharide.

Preferably the cell is transformed to comprise at least one nucleic acid sequence encoding a protein selected from the group comprising, consisting of or consisting essentially of a lactose transporter like e.g., the LacY or lac12 permease, a glucose transporter, a galactose transporter, a transporter for a nucleotide-activated sugar like, for example, a transporter for UDP-GlcNAc, a transporter protein involved in transport of a negatively charged, preferably sialylated, oligosaccharide out of the cell.

In another and/or additional preferred embodiment of the method and/or cell of the disclosure, the cell expresses a membrane transporter protein belonging to the family of MFS transporters like e.g., an MdfA polypeptide of the multidrug transporter MdfA family from species comprising E. coli (UniProt ID P0AEY8), Cronobacter muytjensii (UniProt ID A0A2T7ANQ9), Citrobacter youngae (UniProt ID D4BC23) and Yokenella regensburgei (UniProt ID G9Z5F4). In another preferred embodiment of the method and/or cell of the disclosure, the cell expresses a membrane transporter protein belonging to the family of sugar efflux transporters (SET) like e.g., a SetA polypeptide of the SetA family from species comprising E. coli (UniProt ID P31675, sequence version 03 (11 Oct. 2004)) and Citrobacter koseri (UniProt ID A0A078LM16). In another preferred embodiment of the method and/or cell of the disclosure, the cell expresses a membrane transporter protein belonging to the family of siderophore exporters like e.g., the E. coli entS (UniProt ID P24077, sequence version 02 (1 Nov. 1997)), the K. ascorbata entS (UniProt ID A0A378GQ13) and the E. coli iceT (UniProt ID A0A024L207). In another preferred embodiment of the method and/or cell of the disclosure, the cell expresses a membrane transporter protein belonging to the family of ABC transporters like e.g., oppF from E. coli (UniProt ID P77737), lmrA from Lactococcus lactis subsp. lactis bv. diacetylactis (UniProt ID A0A1V0NEL4) and Blon_2475 from Bifdobacterium longum subsp. infantis (UniProt ID B7GPD4). In a more preferred embodiment of the method and/or cell of the disclosure, the cell expresses more than one membrane transporter protein selected from the list comprising, consisting of or consisting essentially of a lactose transporter like e.g., the LacY or lac12 permease, a fucose transporter, a glucose transporter, a galactose transporter, a transporter for a nucleotide-activated sugar like, for example, a transporter for UDP-GlcNAc, UDP-Gal and/or GDP-Fuc, the MdfA protein from E. coli (UniProt ID POAEY8), the MdfA protein from Cronobacter muytjensii (UniProt ID A0A2T7ANQ9), the MdfA protein from Citrobacter youngae (UniProt ID D4BC23), the MdfA protein from Yokenella regensburgei (UniProt ID G9Z5F4), the SetA protein from E. coli (UniProt ID P31675, sequence version 03 (11 Oct. 2004)), the SetA protein from Citrobacter koseri (UniProt ID A0A078LM16), the entS protein from E. coli (UniProt ID P24077, sequence version 02 (1 Nov. 1997)), the entS protein from K. ascorbata (UniProt ID A0A378GQ13), the iceT protein from E. coli (UniProt ID A0A024L207), the oppF protein from E. coli (UniProt ID P77737), the lmrA protein from Lactococcus lactis subsp. lactis bv. diacetylactis (UniProt ID A0A1V0NEL4) and Blon_2475 from Bifidobacterium longum subsp. infantis (UniProt ID B7GPD4). Preferably the cell is transformed to comprise at least one nucleic acid sequence encoding a membrane transporter protein selected from the list comprising, consisting of or consisting essentially of a siderophore exporter, a major facilitator superfamily (MFS) transporter, an ATP-binding cassette (ABC) transporter or a sugar efflux transporter.

According to another preferred aspect of the method and/or cell of the disclosure, the cell is capable to synthesize N-acetylmannosamine (ManNAc) and/or N-acetylmannosamine-6-phosphate (ManNAc-6-phosphate).

In another and/or additional preferred embodiment of the method and/or cell of the disclosure, one or more gene(s) involved in one or more reductive pathway(s) in the cell is/are rendered less functional or is/are knocked-out. Examples of a reductive pathway comprise but are not limited to the reductive acetyl-CoA-pathway, the reductive pyrimidine catabolic pathway, the reductive citric acid cycle, the thiol-redox pathway and the reductive glycine pathway. In another and/or additional preferred embodiment of the method and/or cell of the disclosure, one or more gene(s) involved in one or more reductive pathway(s) is/are rendered less functional by insertion, deletion and/or modification of one or more nucleotide(s) in one or more polynucleotide sequence(s) selected from the list comprising, consisting of or consisting essentially of promoter sequence, ribosome binding site, untranslated region, coding sequence and transcription terminator sequence of the one or more gene(s). Herein, the one or more gene(s) may be selected from the list comprising, consisting of or consisting essentially of gene(s) encoding formate dehydrogenase, formate-tetrahydrofolate ligase, methenyltetrahydrofolate cyclohydrolase, glycine dehydrogenase/decarboxylating, glycine cleavage system protein, glutamate dehydrogenase, glycine reductase complex, Co² reductase, folate synthetase, folate cyclohydrolase, folate dehydrogenase, folate reductase, methyltransferase, pyruvate synthase, phosphotransacetylase, acetate kinase, ATPase, acetyl-CoA carbonylase/synthase, methylenetetrahydrofolate reductase, methylenetetrahydrofolate dehydrogenase, methyltetrahydrofolate:corrinoid/iron-sulfur protein methyltransferase, methenyltetrahydrofolate cyclohydrolase, pyruvate: ferredoxin oxidoreductase, serine hydroxymethyltransferase, dihydropyrimidine dehydrogenase, dihydropyrimidinase, β-ureidopropionase, ribulose-1,5-bisphosphate carboxylase, ATP citrate lyase, citrate synthase, aconitase, isocitrate dehydrogenase, 2-ketoglutarate dehydrogenase, 2-ketoglutarate ferredoxin oxidoreductase, succinyl-CoA synthetase, succinyl-CoA-acetoacetate-CoA-transferase, fumarate reductase, succinate dehydrogenase, fumarase, malate dehydrogenase, isocitrate lyase, malate synthase, phosphoenol pyruvate carboxykinase, hhosphoenol pyruvate carboxylase, malic enzyme, pyruvate carboxylase/oxaloacetate decarboxylase, pyruvate dehydrogenase, pyruvate ferredoxin oxidoreductase, KatG catalase, alkylhydroperoxide reductase, DNA-binding ferritin-like protein, glutaredoxin, ferric homeostasis regulator, manganese transporter, OxyS, OxyR, glutathione reductase, thioredoxin reductase, thioredoxin-disulphide reductase and glutamine synthetase. Preferably, the one or more genes involved in one or more reductive pathway(s) is/are selected from the list comprising, consisting of or consisting essentially of a glutathione reductase and a thioredoxin reductase.

In a preferred embodiment, the negatively charged, preferably sialylated, oligosaccharide of the disclosure is produced by a cell that is cultured in a cell cultivation. Within the context of the disclosure, the cell cultivation comprises in vitro and/or ex vivo cultivation of cells. In another and/or additional more preferred embodiment, the cell cultivation is a fermentation. In an alternative and/or additional more preferred embodiment, the cell is cultivated or incubated in a reactor as defined herein. In an alternative and/or additional more preferred embodiment, the cell is cultivated or incubated in an incubator as defined herein.

In another and/or additional preferred embodiment, the cell is stably cultured in a cultivation or incubation medium.

In another and/or additional preferred embodiment, the cell is cultivated in cultivation or incubation medium comprising at least one carbon source selected from the list comprising, consisting of or consisting essentially of a monosaccharide; disaccharide; oligosaccharide; polysaccharide; polyol; glycerol; a complex medium including molasses, corn steep liquor, peptone, tryptone or yeast extract. Preferably, the carbon source is selected from the list comprising glucose, N-acetylglucosamine (GlcNAc), glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. More preferably, the at least one carbon source selected from the list consisting of glucose, fructose, sucrose, and glycerol. In a more preferred embodiment, the cultivation or incubation medium is a chemically defined medium. In an additional preferred embodiment, the cultivation or incubation medium is a minimal salt medium comprising sulphate, phosphate, chloride, ammonium, calcium, magnesium, sodium, potassium, iron, copper, zinc, manganese, cobalt, and/or selenium. In another and/or additional preferred embodiment, the cultivation or incubation medium comprises one or more precursor(s) that is/are used for production of the negatively charged, preferably sialylated, oligosaccharide. In another and/or additional preferred embodiment, the cultivation or incubation medium contains at least one compound selected from the list consisting of lactose, galactose, glucose, UDP-galactose (UDP-Gal), sialic acid, CMP-sialic acid, CMP-Neu5Ac and CMP-KDO.

In another and/or additional preferred embodiment of the method of the disclosure, the method for production of a negatively charged, preferably sialylated, oligosaccharide as described herein comprises at least one of the following steps:
i) Use of a cultivation or incubation medium comprising at least one precursor and/or acceptor;
ii) Adding to the cultivation or incubation medium in a reactor or incubator at least one precursor and/or acceptor feed wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of the precursor and/or acceptor feed;
iii) Adding to the cultivation or incubation medium in a reactor or incubator at least one precursor and/or acceptor feed wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of the precursor and/or acceptor feed and wherein preferably, the pH of the precursor and/or acceptor feed is set between 2.0 and 10.0 and wherein preferably, the temperature of the precursor and/or acceptor feed is kept between 20° C. and 80° C.;
iv) Adding at least one precursor and/or acceptor feed in a continuous manner to the cultivation or incubation medium over the course of 1 day, 2 days, 3 days, 4 days, 5 days by way of a precursor and/or acceptor feeding solution;
v) Adding at least one precursor and/or acceptor feed in a continuous manner to the cultivation or incubation medium over the course of 1 day, 2 days, 3 days, 4 days, 5 days by way of a precursor and/or acceptor feeding solution and wherein the concentration of the precursor and/or acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of the precursor and/or acceptor feeding solution is set between 2.0 and 10.0 and wherein preferably, the temperature of the precursor and/or acceptor feeding solution is kept between 20° C. and 80° C.;
said method resulting in the negatively charged, preferably sialylated, oligosaccharide, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. Preferably, the precursor is selected from the list comprising sialic acid, CMP-sialic acid, CMP-Neu5Ac, CMP-KDO, glucose, galactose and UDP-galactose. Preferably, the acceptor is lactose.

In another and/or additional preferred embodiment of the method of the disclosure, the method for production of a negatively charged, preferably sialylated, oligosaccharide as described herein comprises at least one of the following steps:
i) Use of a cultivation or incubation medium comprising at least one precursor and/or acceptor;
ii) Adding to the cultivation or incubation medium in a reactor or incubator at least one precursor and/or acceptor feed in one pulse or in a discontinuous (pulsed) manner wherein the total reactor or incubator volume ranges from 250 mL to 10.000 m³ (cubic meter), and preferably so that the final volume of the cultivation or incubation medium is not more than three-fold, preferably not more than two-fold, more preferably less than 2-fold of the volume of the cultivation or incubation medium before the addition of the precursor and/or acceptor feed pulse(s);
iii) Adding to the cultivation or incubation medium in a reactor or incubator at least one precursor and/or acceptor feed in one pulse or in a discontinuous (pulsed) manner wherein the total reactor or incubator volume ranges from 250 mL to 10.000 m³ (cubic meter), and preferably so that the final volume of the cultivation or incubation medium is not more than three-fold, preferably not more than two-fold, more preferably less than two-fold of the volume of the cultivation or incubation medium before the addition of the precursor and/or acceptor feed pulse(s) and wherein preferably, the pH of the precursor and/or acceptor feed is set between 2.0 and 10.0 and wherein preferably, the temperature of the precursor and/or acceptor feed is kept between 20° C. and 80° C.;
iv) Adding at least one precursor and/or acceptor feed in a discontinuous (pulsed) manner to the cultivation or incubation medium over the course of 1 day, 2 days, 3 days, 4 days, 5 days by way of a precursor and/or acceptor feeding solution;
v) Adding at least one precursor and/or acceptor feed in a discontinuous (pulsed) manner to the cultivation or incubation medium over the course of 1 day, 2 days, 3 days, 4 days, 5 days by way of a precursor and/or acceptor feeding solution and wherein the concentration of the precursor and/or acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of the precursor and/or acceptor feeding solution is set between 2.0 and 10.0 and wherein preferably, the temperature of the precursor and/or acceptor feeding solution is kept between 20° C. and 80° C.;
said method resulting in the negatively charged, preferably sialylated, oligosaccharide, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. Preferably, the precursor is selected from the list comprising sialic acid, CMP-sialic acid, CMP-Neu5Ac, CMP-KDO, glucose, galactose and UDP-galactose. Preferably, the acceptor is lactose.

In another and/or additional preferred embodiment of the method of the disclosure, the method for production of a negatively charged, preferably sialylated, oligosaccharide as described herein comprises at least one of the following steps:
i) Use of a cultivation or incubation medium comprising at least 50, more preferably at least 75, more preferably at least 100, more preferably at least 120, more preferably at least 150 grams of precursor per liter of initial reactor or incubator volume wherein the reactor or incubator volume ranges from 250 mL to 10.000 m³ (cubic meter);
ii) Use of a cultivation or incubation medium comprising at least 50, more preferably at least 75, more preferably at least 100, more preferably at least 120, more preferably at least 150 grams of acceptor per liter of initial reactor or incubator volume wherein the reactor or incubator volume ranges from 250 mL to 10.000 m³ (cubic meter);
iii) Adding to the cultivation or incubation medium in a reactor or incubator a precursor feed comprising at least 50, more preferably at least 75, more preferably at least 100, more preferably at least 120, more preferably at least 150 grams of precursor per liter of initial reactor or incubator volume wherein the reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of the precursor feed;
iv) Adding to the cultivation or incubation medium in a reactor or incubator an acceptor feed comprising at least 50, more preferably at least 75, more preferably at least 100, more preferably at least 120, more preferably at least 150 grams of acceptor per liter of initial reactor or incubator volume wherein the reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of the acceptor feed;
v) Adding to the cultivation or incubation medium a precursor feed comprising at least 50, more preferably at least 75, more preferably at least 100, more preferably at least 120, more preferably at least 150 grams of precursor per liter of initial reactor or incubator volume wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of the precursor feed and wherein preferably, the pH of the precursor feed is set between 2.0 and 10.0 and wherein preferably, the temperature of the precursor feed is kept between 20° C. and 80° C.;
vi) Adding to the cultivation or incubation medium an acceptor feed comprising at least 50, more preferably at least 75, more preferably at least 100, more preferably at least 120, more preferably at least 150 grams of acceptor per liter of initial reactor or incubator volume wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of the acceptor feed and wherein preferably, the pH of the acceptor feed is set between 2.0 and 10.0 and wherein preferably, the temperature of the acceptor feed is kept between 20° C. and 80° C.;
vii) Adding a precursor and/or acceptor feed in a continuous manner to the cultivation or incubation medium over the course of 1 day, 2 days, 3 days, 4 days, 5 days by way of a precursor and/or acceptor feeding solution;
viii) Adding a precursor feed in a continuous manner to the cultivation or incubation medium over the course of 1 day, 2 days, 3 days, 4 days, 5 days by way of a precursor feeding solution and wherein the concentration of the precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the precursor feeding solution is set between 2.0 and 10.0 and wherein preferably, the temperature of the precursor feeding solution is kept between 20° C. and 80° C.;
ix) Adding an acceptor feed in a continuous manner to the cultivation or incubation medium over the course of 1 day, 2 days, 3 days, 4 days, 5 days by way of an acceptor feeding solution and wherein the concentration of the acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the acceptor feeding solution is set between 2.0 and 10.0 and wherein preferably, the temperature of the acceptor feeding solution is kept between 20° C. and 80° C.;
said method resulting in the negatively charged, preferably sialylated, oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. Preferably, the precursor is selected from the list comprising sialic acid, CMP-sialic acid, CMP-Neu5Ac, CMP-KDO, glucose, galactose and UDP-galactose. Preferably, the acceptor is lactose.

In another and/or additional preferred embodiment of the method of the disclosure, the method for production of a negatively charged, preferably sialylated, oligosaccharide as described herein comprises at least one of the following steps:
i) Use of a cultivation or incubation medium comprising at least 50, more preferably at least 75, more preferably at least 100, more preferably at least 120, more preferably at least 150 grams of precursor per liter of initial reactor or incubator volume wherein the reactor or incubator volume ranges from 250 mL to 10.000 m³ (cubic meter);
ii) Use of a cultivation or incubation medium comprising at least 50, more preferably at least 75, more preferably at least 100, more preferably at least 120, more preferably at least 150 grams of acceptor per liter of initial reactor or incubator volume wherein the reactor or incubator volume ranges from 250 mL to 10.000 m³ (cubic meter);
iii) Adding to the cultivation or incubation medium in a reactor or incubator a precursor feed comprising at least 50, more preferably at least 75, more preferably at least 100, more preferably at least 120, more preferably at least 150 grams of precursor per liter of initial reactor or incubator volume wherein the reactor or incubator volume ranges from 250 mL to 10.000 m³ (cubic meter) in one pulse or in a discontinuous (pulsed) manner, and preferably so that the final volume of the cultivation or incubation medium is not more than three-fold, preferably not more than two-fold, more preferably less than 2-fold of the volume of the cultivation or incubation medium before the addition of the precursor feed;
iv) Adding to the cultivation or incubation medium in a reactor or incubator an acceptor feed comprising at least 50, more preferably at least 75, more preferably at least 100, more preferably at least 120, more preferably at least 150 grams of acceptor per liter of initial reactor or incubator volume wherein the reactor or incubator volume ranges from 250 mL to 10.000 m³ (cubic meter) in one pulse or in a discontinuous (pulsed) manner, and preferably so that the final volume of the cultivation or incubation medium is not more than three-fold, preferably not more than two-fold, more preferably less than 2-fold of the volume of the cultivation or incubation medium before the addition of the acceptor feed;
v) Adding to the cultivation or incubation medium a precursor feed comprising at least 50, more preferably at least 75, more preferably at least 100, more preferably at least 120, more preferably at least 150 grams of precursor per liter of initial reactor or incubator volume wherein the total reactor or incubator volume ranges from 250 mL to 10.000 m³ (cubic meter) in one pulse or in a discontinuous (pulsed) manner, and preferably so that the final volume of the cultivation or incubation medium is not more than three-fold, preferably not more than two-fold, more preferably less than 2-fold of the volume of the cultivation or incubation medium before the addition of the precursor feed and wherein preferably, the pH of the precursor feed is set between 2.0 and 10.0 and wherein preferably, the temperature of the precursor feed is kept between 20° C. and 80° C.;
vi) Adding to the cultivation or incubation medium an acceptor feed comprising at least 50, more preferably at least 75, more preferably at least 100, more preferably at least 120, more preferably at least 150 grams of acceptor per liter of initial reactor or incubator volume wherein the total reactor or incubator volume ranges from 250 mL to 10.000 m³ (cubic meter) in one pulse or in a discontinuous (pulsed) manner, and preferably so that the final volume of the cultivation or incubation medium is not more than three-fold, preferably not more than two-fold, more preferably less than 2-fold of the volume of the cultivation or incubation medium before the addition of the acceptor feed and wherein preferably, the pH of the acceptor feed is set between 2.0 and 10.0 and wherein preferably, the temperature of the acceptor feed is kept between 20° C. and 80° C.;
vii) Adding a precursor and/or acceptor feed in a discontinuous (pulsed) manner to the cultivation or incubation medium over the course of 1 day, 2 days, 3 days, 4 days, 5 days by way of a precursor and/or acceptor feeding solution;
viii) Adding a precursor feed in a discontinuous (pulsed) manner to the cultivation or incubation medium over the course of 1 day, 2 days, 3 days, 4 days, 5 days by way of a precursor feeding solution and wherein the concentration of the precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the precursor feeding solution is set between 2.0 and 10.0 and wherein preferably, the temperature of the precursor feeding solution is kept between 20° C. and 80° C.;
ix) Adding an acceptor feed in a discontinuous (pulsed) manner to the cultivation or incubation medium over the course of 1 day, 2 days, 3 days, 4 days, 5 days by way of an acceptor feeding solution and wherein the concentration of the acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the acceptor feeding solution is set between 2.0 and 10.0 and wherein preferably, the temperature of the acceptor feeding solution is kept between 20° C. and 80° C.;
said method resulting in the negatively charged, preferably sialylated, oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation. Preferably, the precursor is selected from the list comprising sialic acid, CMP-sialic acid, CMP-Neu5Ac, CMP-KDO, glucose, galactose and UDP-galactose. Preferably, the acceptor is lactose.

In a further, more preferred embodiment, the method for the production of a negatively charged, preferably sialylated, oligosaccharide as described herein comprises at least one of the following steps:
i) Adding to the cultivation or incubation medium a lactose feed comprising at least 50, more preferably at least 75, more preferably at least 100, more preferably at least 120, more preferably at least 150 gram of lactose per liter of initial reactor volume wherein the total reactor volume ranges from 250 mL (millilitre) to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of the lactose feed;
ii) Adding a lactose feed in a continuous manner to the cultivation or incubation medium over the course of 1 day, 2 days, 3 days, 4 days, 5 days by way of a feeding solution;
iii) Adding a lactose feed in a continuous manner to the cultivation or incubation medium over the course of 1 day, 2 days, 3 days, 4 days, 5 days by way of a feeding solution and wherein the concentration of the lactose feeding solution is 25 g/L, preferably 50 g/L, more preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the solution is set between 3 and 7 and
wherein preferably the temperature of the feed solution is kept between 20° C. and 80° C.;
said method resulting in a negatively charged, preferably sialylated, oligosaccharide with a concentration of at least 30 g/L, at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation.

Preferably the lactose feed is accomplished by adding lactose from the beginning of the cultivation or incubation in a concentration of at least 1 mM, preferably at least 5 mM, preferably in a concentration of 30, 40, 50, 60, 70, 80, 90, 100, 150 mM, more preferably in a concentration >300 mM.

In another aspect the lactose feed is accomplished by adding lactose to the cultivation or incubation medium in a concentration, such that throughout the production phase of the cultivation or incubation a lactose concentration of at least 1 mM, preferably 5 mM, 10 mM or 30 mM is obtained.

In a further embodiment of the methods described herein the cells are cultivated or incubated for at least about 60, 80, 100, or about 120 hours or in a continuous manner.

In a preferred embodiment, a carbon source is provided, preferably sucrose, in the cultivation or incubation medium for 3 or more days, preferably up to 7 days; and/or provided, in the cultivation or incubation medium, at least 100, advantageously at least 105, more advantageously at least 110, even more advantageously at least 120 grams of sucrose per liter of initial cultivation or incubation medium in a continuous manner, so that the final volume of the cultivation or incubation medium is not more than three-fold, advantageously not more than two-fold, more advantageously less than two-fold of the volume of the cultivation or incubation medium before the culturing.

Preferably, when performing the method as described herein, a first phase of exponential cell growth is provided by adding a carbon source, preferably glucose or sucrose, to the cultivation or incubation medium before the lactose is added to the cultivation or incubation medium in a second phase.

In an alternative preferable embodiment, in the method as described herein, the lactose is added already in the first phase of exponential growth together with the carbon-based substrate.

In another and/or additional preferred embodiment of the method and/or cell of the disclosure, the cell is selected from the list consisting of prokaryotic cells and eukaryotic cells, preferably from the list consisting of yeast cells, bacterial cells, archaebacterial cells, algae cells, plant cells, fungal cells, animal cells and protozoan cells. In another preferred embodiment, the cell is a bacterium, fungus, yeast, a plant cell, an animal cell or a protozoan cell.

The latter bacterium preferably belongs to the phylum of the Proteobacteria or the phylum of the Firmicutes or the phylum of the Cyanobacteria or the phylum Deinococcus-Thermus or the phylum of Actinobacteria. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, the disclosure specifically relates to a mutated and/or transformed Escherichia coli cell or strain as indicated above wherein the E. coli strain is a K12 strain. More preferably, the Escherichia coli K12 strain is E. coli MG1655. The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter bacterium belonging to the phylum Proteobacteria, preferably belonging to the family of the Vibrionaceae, with member Vibrio natriegens. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like e.g., Saccharomyces cerevisiae, S. bayanus, S. boulardii), Zygosaccharomyces, Pichia (with members like e.g., Pichia pastoris, P. anomala, P. kluyveri), Komagataella, Hansenula, Kluyveromyces (with members like e.g., Kluyveromyces lactis, K. marxianus, K. thermotolerans), Debaromyces, Candida, Schizosaccharomyces, Schwanniomyces, Torulaspora, Yarrowia (like e.g., Yarrowia lipolytica) or Starmerella (like e.g., Starmerella bombicola). The latter yeast is preferably selected from Pichia pastoris, Yarrowia lipolytica, Saccharomyces cerevisiae, Kluyveromyces lactis, Hansenula polymorpha, Kluyveromyces marxianus, Pichia methanolica, Pichia stipites, Candida boidinii, Schizosaccharomyces pombe, Schwanniomyces occidentalis, Torulaspora delbrueckii, Zygosaccharomyces rouxii, and Zygosaccharomyces bailii. The latter fungus belongs preferably to the genus Rhizopus, Dictyostelium, Penicillium, Mucor or Aspergillus. Plant cells include cells of flowering and non-flowering plants, as well as algal cells, for example, Chlamydomonas, Chlorella, etc. Preferably, the plant is a tobacco, rose, alfalfa, rice, tomato, cotton, rapeseed, soy, maize, or corn plant. More preferably, the latter plant cell is selected from the Rosa family. The latter animal cell is preferably derived from non-human mammals (e.g., cattle, buffalo, pig, sheep, mouse, rat, primate (e.g., chimpanzee, orangutan, gorilla, monkey (e.g., Old World, New World), lemur), dog, cat, rabbit, horse, cow, goat, ox, deer, musk deer, bovid, whale, dolphin, hippopotamus, elephant, rhinoceros, giraffe, zebra, lion, cheetah, tiger, panda, red panda, otter), birds (e.g., chicken, duck, ostrich, turkey, pheasant), fish (e.g., swordfish, salmon, tuna, sea bass, trout, catfish), invertebrates (e.g., lobster, crab, shrimp, clams, oyster, mussel, sea urchin), reptiles (e.g., snake, alligator, turtle), amphibians (e.g., frogs) or insects (e.g., fly, nematode) or is a genetically modified cell line derived from human cells excluding embryonic stem cells. Both human and non-human mammalian cells are preferably selected from the list comprising an epithelial cell like e.g., a mammary epithelial cell, an embryonic kidney cell (e.g., HEK293 or HEK 293T cell), a fibroblast cell, a COS cell, a Chinese hamster ovary (CHO) cell, a murine myeloma cell like e.g., an N20, SP2/0 or YB2/0 cell, an NIH-3T3 cell, a non-mammary adult stem cell or derivatives thereof such as described in WO21067641, preferably mesenchymal stem cell or derivates thereof as described in WO21067641, a lactocyte derived from mammalian induced pluripotent stem cells, preferably human induced pluripotent stem cells, a lactocyte as part of mammary-like gland organoids, a post-parturition mammary epithelium cell, a polarized mammary cell, preferably a polarized mammary cell selected from the list comprising live primary mammary epithelial cells, live mammary myoepithelial cells, live mammary progenitor cells, live immortalized mammary epithelial cells, live immortalized mammary myoepithelial cells, live immortalized mammary progenitor cells, a non-mammary adult stem cell or derivatives thereof as well-known to the person skilled in the art from e.g., WO2021/219634, WO 2022/054053, WO 2021/141762, WO 2021/142241, WO 2021/067641 and WO2021/242866. The latter insect cell is preferably derived from Spodoptera frugiperda like e.g., Sf9 or Sf21 cells, Bombyx mori, Mamestra brassicae, Trichoplusia ni like e.g., BTI-TN-5B1-4 cells or Drosophila melanogaster like e.g., Drosophila S2 cells. The latter protozoan cell preferably is a Leishmania tarentolae cell.

In another and/or additional preferred embodiment, the cell is an E. coli or yeast with a lactose permease positive phenotype, preferably wherein the lactose permease is coded by the gene LacY or LAC12, respectively.

In another and/or additional preferred embodiment, the cell is a viable Gram-negative bacterium that comprises a reduced or abolished synthesis of poly-N-acetylglucosamine (PNAG), Enterobacterial Common Antigen (ECA), cellulose, colanic acid, core oligosaccharides, Osmoregulated Periplasmic Glucans (OPG), glucosylglycerol, glycan and/or trehalose compared to a non-modified progenitor.

In a more preferred embodiment of the method and/or cell, the reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose is provided by a mutation in any one or more glycosyltransferases involved in the synthesis of any one of the poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose, wherein the mutation provides for a deletion or lower expression of any one of the glycosyltransferases. The glycosyltransferases comprise glycosyltransferase genes encoding poly-N-acetyl-D-glucosamine synthase subunits, UDP-N-acetylglucosamine-undecaprenyl-phosphate N-acetylglucosaminephosphotransferase, Fuc4NAc (4-acetamido-4,6-dideoxy-D-galactose) transferase, UDP-N-acetyl-D-mannosaminuronic acid transferase, the glycosyltransferase genes encoding the cellulose synthase catalytic subunits, the cellulose biosynthesis protein, colanic acid biosynthesis glucuronosyltransferase, colanic acid biosynthesis galactosyltransferase, colanic acid biosynthesis fucosyltransferase, UDP-glucose:undecaprenyl-phosphate glucose-1-phosphate transferase, putative colanic biosynthesis glycosyl transferase, UDP-glucuronate:LPS(HepIII) glycosyltransferase, ADP-heptose—LPS heptosyltransferase 2, ADP-heptose:LPS heptosyltransferase 1, putative ADP-heptose:LPS heptosyltransferase 4, lipopolysaccharide core biosynthesis protein, UDP-glucose:(glucosyl)LPS α-1,2-glucosyltransferase, UDP-D-glucose:(glucosyl)LPS α-1,3-glucosyltransferase, UDP-D-galactose:(glucosyl)lipopolysaccharide-1,6-D-galactosyltransferase, lipopolysaccharide glucosyltransferase I, lipopolysaccharide core heptosyltransferase 3, β-1,6-galactofuranosyltransferase, undecaprenyl-phosphate 4-deoxy-4-formamido-L-arabinose transferase, lipid IVA 4-amino-4-deoxy-L-arabinosyltransferase, bactoprenol glucosyl transferase, putative family 2 glycosyltransferase, the osmoregulated periplasmic glucans (OPG) biosynthesis protein G, OPG biosynthesis protein H, glucosylglycerate phosphorylase, glycogen synthase, 1,4-α-glucan branching enzyme, 4-α-glucanotransferase and trehalose-6-phosphate synthase. In an exemplary embodiment, the cell is mutated in any one or more of the glycosyltransferases comprising pgaC, pgaD, rfe, rffT, rffM, bcsA, bcsB, bcsC, wcaA, wcaC, wcaE, weal, wcaJ, wcaL, waaH, waaF, waaC, waaU, waaZ, waaJ, waaO, waaB, waaS, waaG, waaQ, wbbl, arnC, arnT, yfdH, wbbK, opgG, opgH, ycjM, glgA, glgB, malQ, otsA and yaiP, wherein the mutation provides for a deletion or lower expression of any one of the glycosyltransferases.

In an alternative and/or additional preferred embodiment of the method and/or cell, the reduced or abolished synthesis of poly-N-acetyl-glucosamine (PNAG) is provided by over-expression of a carbon storage regulator encoding gene, deletion of a Na+/H+ antiporter regulator encoding gene and/or deletion of the sensor histidine kinase encoding gene.

In another and/or additional preferred embodiment of the method and/or cell of the disclosure, the cell produces 30 g/L or more of the negatively charged, preferably sialylated, oligosaccharide in the whole broth and/or supernatant and/or wherein the negatively charged, preferably sialylated, oligosaccharide in the whole broth and/or supernatant has a purity of at least 80% measured on the total amount of negatively charged, preferably sialylated, oligosaccharide and its precursor(s) produced by the cell in the whole broth and/or supernatant, respectively. In a more preferred embodiment, the cell produces 30 g/L, 31 g/L, 32 g/L, 33 g/L, 34 g/L, 35 g/L, 36 g/L, 37 g/L, 38 g/L, 39 g/L, 40 g/L, 41 g/L, 42 g/L, 43 g/L, 44 g/L, 45 g/L, 46 g/L, 47 g/L, 48 g/L, 49 g/L, 50 g/L, 51 g/L, 52 g/L, 53 g/L, 54 g/L, 55 g/L, 56 g/L, 57 g/L, 58 g/L, 59 g/L, 60 g/L, 61 g/L, 62 g/L, 63 g/L, 64 g/L, 65 g/L, 66 g/L, 67 g/L, 68 g/L, 69 g/L, 70 g/L, 71 g/L, 72 g/L, 73 g/L, 74 g/L, 75 g/L, 76 g/L, 77 g/L, 78 g/L, 79 g/L, 80 g/L, 81 g/L, 82 g/L, 83 g/L, 84 g/L, 85 g/L or more than 85 g/L of the negatively charged, preferably sialylated, oligosaccharide in the whole broth and/or supernatant.

In another aspect of the disclosure, the cell produces a negatively charged, preferably sialylated, oligosaccharide as described herein.

In a preferred embodiment of the method and/or cell of the disclosure, the cell produces an oligosaccharide mixture comprising a negatively charged, preferably sialylated, oligosaccharide as described herein. In another and/or additional preferred embodiment of the method and/or cell of the disclosure, the cell is capable to produce and/or produces a mixture of two or more oligosaccharides wherein at least one of the two or more oligosaccharides is a negatively charged, preferably sialylated, oligosaccharide as described herein. In another and/or additional preferred embodiment of the method and/or cell of the disclosure, the cell is capable to produce and/or produces a mixture of one or more disaccharide(s) and one or more oligosaccharide(s) wherein at least one of the one or more oligosaccharide(s) is a negatively charged, preferably sialylated, oligosaccharide as described herein. In another and/or additional preferred embodiment of the method and/or cell of the disclosure, the cell is capable to produce and/or produces a mixture comprising a negatively charged, preferably sialylated, oligosaccharide and at least one non-charged (neutral) oligosaccharide. In another and/or additional preferred embodiment of the method and/or cell of the disclosure, the cell is capable to produce and/or produces a mixture comprising 1) a negatively charged, preferably sialylated, oligosaccharide and 2) a disaccharide and/or a non-charged (neutral) oligosaccharide.

In another aspect of the disclosure, the negatively charged, preferably sialylated, oligosaccharide produced by a cell of the disclosure is recovered from the cultivation or incubation medium and/or the cell. In a preferred embodiment, the negatively charged, preferably sialylated, oligosaccharide is purified.

The terms “separating from the cultivation or incubation” means harvesting, collecting, or retrieving the negatively charged, preferably sialylated, oligosaccharide from the cell and/or the medium of its growth.

The negatively charged, preferably sialylated, oligosaccharide can be separated in a conventional manner from the aqueous culture, cultivation or incubation medium, in which the cell was grown. In case the negatively charged, preferably sialylated, oligosaccharide is still present in the cells producing the negatively charged, preferably sialylated, oligosaccharide, conventional manners to free or to extract the oligosaccharide out of the cells can be used, such as cell destruction using high pH, heat shock, sonication, French press, homogenization, enzymatic hydrolysis, chemical hydrolysis, solvent hydrolysis, detergent, hydrolysis, . . . . The culture, cultivation or incubation medium and/or cell extract together and separately can then be further used for separating the negatively charged, preferably sialylated, oligosaccharide.

This preferably involves clarifying the negatively charged, preferably sialylated, oligosaccharide to remove suspended particulates and contaminants, particularly cells, cell components, insoluble metabolites and debris produced by culturing the genetically engineered cell. In this step, the negatively charged, preferably sialylated, oligosaccharide can be clarified in a conventional manner. Preferably, the negatively charged, preferably sialylated, oligosaccharide is clarified by centrifugation, flocculation, decantation and/or filtration. A second step of separating the negatively charged, preferably sialylated, oligosaccharide preferably involves removing substantially all the eventually remaining proteins, peptides, amino acids, RNA, DNA, endotoxins and glycolipids that could interfere with the subsequent separation step, from the negatively charged, preferably sialylated, oligosaccharide, preferably after it has been clarified. In this step, remaining proteins and related impurities can be removed from the negatively charged, preferably sialylated, oligosaccharide in a conventional manner. Preferably, remaining proteins, salts, by-products, color, endotoxins and other related impurities are removed from the negatively charged, preferably sialylated, oligosaccharide by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g., using slab-polyacrylamide or sodium dodecyl sulphate-polyacrylamide gel electrophoresis (PAGE)), affinity chromatography (using affinity ligands including e.g., DEAE-sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography, electrodialysis and/or gel filtration (i.e., size exclusion chromatography), particularly by chromatography, more particularly by ion exchange chromatography or hydrophobic interaction chromatography or ligand exchange chromatography. With the exception of size exclusion chromatography, remaining proteins and related impurities are retained by a chromatography medium or a selected membrane.

In a further preferred embodiment, the methods as described herein also provide for a further purification of the negatively charged, preferably sialylated, oligosaccharide of the disclosure. A further purification of the negatively charged, preferably sialylated, oligosaccharide may be accomplished, for example, by use of (activated) charcoal or carbon, nanofiltration, ultrafiltration, electrophoresis, enzymatic treatment, ion exchange, temperature adjustment, pH adjustment or pH adjustment with an alkaline or acidic solution to remove any remaining DNA, protein, LPS, endotoxins, or other impurity. Alcohols, such as ethanol, and aqueous alcohol mixtures can also be used. Another purification step is accomplished by crystallization, evaporation or precipitation of the negatively charged, preferably sialylated, oligosaccharide. Another purification step is to dry, e.g., spray dry, lyophilize, spray freeze dry, freeze spray dry, band dry, belt dry, vacuum band dry, vacuum belt dry, drum dry, roller dry, vacuum drum dry or vacuum roller dry the produced negatively charged, preferably sialylated, oligosaccharide.

In an exemplary embodiment, the separation and purification of the negatively charged, preferably sialylated, oligosaccharide is made in a process, comprising the following steps in any order:
a) contacting the cultivation or a clarified version thereof with a nanofiltration membrane with a molecular weight cut-off (MWCO) of 600-3500 Da ensuring the retention of the produced negatively charged, preferably sialylated, oligosaccharide and allowing at least a part of the proteins, salts, by-products, color and other related impurities to pass,
b) conducting a diafiltration process on the retentate from step a), using the membrane, with an aqueous solution of an inorganic electrolyte, followed by optional diafiltration with pure water to remove excess of the electrolyte,
c) and collecting the retentate enriched in the negatively charged, preferably sialylated, oligosaccharide in the form of a salt from the cation of the electrolyte.

In an alternative exemplary embodiment, the separation and purification of the negatively charged, preferably sialylated, oligosaccharide is made in a process, comprising the following steps in any order: subjecting the cultivation or a clarified version thereof to two membrane filtration steps using different membranes, wherein
one membrane has a molecular weight cut-off of between about 300 to about 500 Dalton, and
the other membrane as a molecular weight cut-off of between about 600 to about 800 Dalton.

In an alternative exemplary embodiment, the separation and purification of the negatively charged, preferably sialylated, oligosaccharide is made in a process, comprising the following steps in any order comprising the step of treating the cultivation or a clarified version thereof with a strong cation exchange resin in H+-form and a weak anion exchange resin in free base form.

In an alternative exemplary embodiment, the separation and purification of the negatively charged, preferably sialylated, oligosaccharide is made in the following way. The cultivation comprising the produced negatively charged, preferably sialylated, oligosaccharide, biomass, medium components and contaminants, and wherein the purity of the produced negatively charged, preferably sialylated, oligosaccharide in the cultivation is <80%, is applied to the following purification steps:
i) separation of biomass from the cultivation,
ii) cationic ion exchanger treatment for the removal of positively charged material,
iii) anionic ion exchanger treatment for the removal of negatively charged material,
iv) nanofiltration step and/or electrodialysis step,
wherein a purified solution comprising the produced negatively charged, preferably sialylated, oligosaccharide at a purity of greater than or equal to 80% is provided. Optionally the purified solution is spray dried.

In an alternative exemplary embodiment, the separation and purification of the negatively charged, preferably sialylated, oligosaccharide is made in a process, comprising the following steps in any order: enzymatic treatment of the cultivation; removal of the biomass from the cultivation; ultrafiltration; nanofiltration; and a column chromatography step. Preferably such column chromatography is a single column or a multiple column. Further preferably the column chromatography step is simulated moving bed chromatography. Such simulated moving bed chromatography preferably comprises i) at least 4 columns, wherein at least one column comprises a weak or strong cation exchange resin; and/or ii) four zones I, II, III and IV with different flow rates; and/or iii) an eluent comprising water; and/or iv) an operating temperature of 15 degrees to 60 degrees centigrade.

In a specific embodiment, the disclosure provides the produced negatively charged, preferably sialylated, oligosaccharide, which is spray-dried to powder, wherein the spray-dried powder contains <15%-wt. of water, preferably <10%-wt. of water, more preferably <7%-wt. of water, most preferably <5%-wt. of water.

For identification of the negatively charged, preferably sialylated, oligosaccharide as described herein, the monomeric building blocks (e.g., the monosaccharide or glycan unit composition), the anomeric configuration of side chains, the presence and location of substituent groups, degree of polymerization/molecular weight and the linkage pattern can be identified by standard methods known in the art, such as, e.g., methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the negatively charged, preferably sialylated, oligosaccharide methods such as e.g., acid-catalyzed hydrolysis, HPLC (high performance liquid chromatography) or GLC (gas-liquid chromatography) (after conversion to alditol acetates) may be used. To determine the glycosidic linkages, the negatively charged, preferably sialylated, oligosaccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, the negatively charged, preferably sialylated, oligosaccharide is subjected to enzymatic analysis, e.g., it is contacted with an enzyme that is specific for a particular type of linkage, e.g., beta-galactosidase, or alpha-glucosidase, etc., and NMR may be used to analyze the products.

In another aspect, the disclosure provides use of a cell as described herein for the production of a negatively charged, preferably sialylated, oligosaccharide as described herein.

In another aspect, the disclosure provides use of a method as described herein for the production of a negatively charged, preferably sialylated, oligosaccharide as described herein.

In another aspect, the disclosure provides for a purified negatively charged, preferably sialylated, oligosaccharide, or a purified oligosaccharide mixture comprising a negatively charged, preferably sialylated, oligosaccharide as described herein for use in medicine, preferably for use in prophylaxis or therapy of a gastrointestinal disorder.

In another aspect, the disclosure provides use of a purified negatively charged, preferably sialylated, oligosaccharide obtained by a method as described herein in a food or feed preparation, in a dietary supplement, in a cosmetic ingredient or in a pharmaceutical ingredient. In some embodiments, the negatively charged, preferably sialylated, oligosaccharide is mixed with one or more ingredients suitable for food, feed, dietary supplement, pharmaceutical ingredient, cosmetic ingredient or medicine. The purified negatively charged, preferably sialylated, oligosaccharide may be used for the manufacture of a preparation, as food additive, prebiotic, symbiotic, for the supplementation of baby food, adult food, infant animal feed, adult animal feed, or as either therapeutically or pharmaceutically active compound or in cosmetic applications. In another aspect, the disclosure provides use of milk oligosaccharide as described herein as additive in food, preferably as additive in human food and/or pet food, more preferably as additive in human baby food. In the context of the disclosure, the food is a human food, preferably infant food, human baby food and/or an infant formula or an infant supplement and the feed is a pet food, animal milk replacer, veterinary product, veterinary feed supplement, nutrition supplement, post weaning feed, or creep feed.

In another preferred embodiment, a preparation is provided that further comprises at least one probiotic microorganism. In another preferred embodiment of the disclosure, the preparation is a nutritional composition. In a more preferred embodiment, the preparation is a medicinal formulation, a dietary supplement, a dairy drink or an infant formula. A “prebiotic” is a substance that promotes growth of microorganisms beneficial to the host, particularly microorganisms in the gastrointestinal tract. In some embodiments, a dietary supplement provides multiple prebiotics, including the negatively charged, preferably sialylated, oligosaccharide being a prebiotic purified by a method disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, the negatively charged, preferably sialylated, oligosaccharide produced and/or purified by a method of this specification is orally administered in combination with such microorganism. Examples of further ingredients for dietary supplements include oligosaccharides (such as 2′-fucosyllactose, 3-fucosyllactose, 3′-sialyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavorings.

In some embodiments, the negatively charged, preferably sialylated, oligosaccharide purified by a method as described herein is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, the negatively charged, preferably sialylated, oligosaccharide purified by a method as described herein is included in infant formula to provide nutritional benefits similar to those provided by the oligosaccharides in human breast milk. In some embodiments, the purified negatively charged, preferably sialylated, oligosaccharide is mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, Bb, Bi2, C and D), minerals (such as potassium citrate, calcium citrate, magnesium chloride, sodium chloride, sodium citrate and calcium phosphate) and possibly human milk oligosaccharides (HMOs). In some embodiments, the one or more infant formula ingredients comprise non-fat milk, a carbohydrate source, a protein source, a fat source, and/or a vitamin and mineral. In some embodiments, the one or more infant formula ingredients comprise lactose, whey protein concentrate and/or high oleic safflower oil. In some embodiments, the concentration of the negatively charged, preferably sialylated, oligosaccharide in the infant formula is approximately the same concentration as the concentration of the oligosaccharide generally present in human breast milk. In some embodiments, a negatively charged, preferably sialylated, oligosaccharide purified by a method as described herein is added to the infant formula with a concentration that is approximately the same concentration as the concentration of the compound generally present in human breast milk.

As will be shown in the examples herein, the methods and the cell of the disclosure preferably provide at least one of the following further surprising advantages when using a hydrolyzing UDP-N-acetyl-D-glucosamine-2-epimerase as described herein:
Higher titers of the negatively charged, preferably sialylated, oligosaccharide (g/L) as described herein,
Higher purity of the negatively charged, preferably sialylated, oligosaccharide (g/L),
A purity of the negatively charged, preferably sialylated, oligosaccharide (g/L) equal to or greater than 80%, preferably equal to or greater than 85%, more preferably equal to or greater than 90%, even more preferably equal to or greater than 91%, even more preferably equal to or greater than 92%, even more preferably equal to or greater than 93%, even more preferably equal to or greater than 94%, even more preferably equal to or greater than 95%, even more preferably equal to or greater than 96%, even more preferably equal to or greater than 97%, even more preferably equal to or greater than 98%, even more preferably equal to or greater than 99%,
Higher lactose conversion, leading to lower lactose concentration at end of fermentation (g/L lactose),
Lower sialic acid formation (g/L),
Higher production rate r (g negatively charged, preferably sialylated, oligosaccharide/L/h),
Higher cell performance index CPI (g negatively charged, preferably sialylated, oligosaccharide/g X),
Higher specific productivity Qp (g negatively charged, preferably sialylated, oligosaccharide/g X/h),
Higher yield on the carbon source used Y (g negatively charged, preferably sialylated, oligosaccharide/g carbon source used),
Higher yield on sucrose Ys (g negatively charged, preferably sialylated, oligosaccharide/g sucrose),
Higher uptake/conversion rate of the carbon source used Q (g carbon source/g X/h),
Higher sucrose uptake/conversion rate Qs (g sucrose/g X/h),
Higher lactose conversion/consumption rate rs (g lactose/h),
Higher secretion, excretion or extracellular transport of the negatively charged, preferably sialylated, oligosaccharide,
Higher growth speed of the production host,
Better fine-tuning of the titer of the negatively charged, preferably sialylated, oligosaccharide (g/L) in a mixture of two or more oligosaccharides,
Better fine-tuning of the titer of the negatively charged, preferably sialylated, oligosaccharide (g/L) in a mixture of one or more disaccharide(s) and one or more oligosaccharide(s),
Higher purity of the negatively charged, preferably sialylated, oligosaccharide (g/L) in a mixture of two or more oligosaccharides,
Higher purity of the negatively charged, preferably sialylated, oligosaccharide (g/L) in a mixture of one or more disaccharide(s) and one or more oligosaccharide(s),
Smaller titers of any one or more of GlcNAc, ManNAc, N-acetyllactosamine (LacNAc), lacto-N-biose (LNB), sialylated GlcNAc, sialylated ManNAc, sialylated LacNAc, sialylated LNB (g/L) in a mixture comprising the negatively, preferably sialylated, oligosaccharide,
Smaller titers of any one or more of GlcNAc, ManNAc, N-acetyllactosamine (LacNAc), lacto-N-biose (LNB), sialylated GlcNAc, sialylated ManNAc, sialylated LacNAc, sialylated LNB (g/L) in a mixture of two or more oligosaccharides comprising the negatively, preferably sialylated, oligosaccharide, and/or
Smaller titers of any one or more of GlcNAc, ManNAc, N-acetyllactosamine (LacNAc), lacto-N-biose (LNB), sialylated GlcNAc, sialylated ManNAc, sialylated LacNAc, sialylated LNB (g/L) in a mixture of one or more disaccharide(s) and one or more oligosaccharide(s) comprising the negatively, preferably sialylated, oligosaccharide,
when compared to a method or a cell using an identical setup or enzymatic or genetic background but lacking the use of a hydrolyzing UDP-N-acetyl-D-glucosamine-2-epimerase as described herein.

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features that are still to be explained below can be used not only in the respectively specified combinations, but also in other combinations or on their own, without departing from the scope of the disclosure.

Moreover, the disclosure relates to the following specific embodiments:
1. A cell comprising a pathway for production of a negatively charged, preferably sialylated, oligosaccharide, characterized in that the cell is genetically engineered to possess or express, preferably to over-express, a hydrolyzing UDP-N-acetyl-D-glucosamine-2-epimerase wherein the hydrolyzing UDP-N-acetyl-D-glucosamine-2-epimerase comprises an amino acid sequence comprising a conserved motif with SEQ ID NO: 12 and having hydrolyzing UDP-N-acetyl-D-glucosamine-2-epimerase activity.
2. Cell according to embodiment 1, wherein the hydrolyzing UDP-N-acetyl-D-glucosamine-2-epimerase comprises, consists or consists essentially of an amino acid sequence that is at least 80% identical over a stretch of at least 150 amino acid residues, preferably at least 200 amino acid residues, to any one of the amino acid sequences as represented by SEQ ID NOs:01, 02, 03, 04, 05, 06, 07 or 08.
3. Cell according to any one of embodiment 1 or 2, wherein the hydrolyzing UDP-N-acetyl-D-glucosamine-2-epimerase comprises an amino acid sequence:
   comprising an IPR domain selected from the list comprising IPR003331, IPR020004 and IPR029767 as defined by InterPro 90.0 as released on 4 Aug. 2022,
   comprising a PF02350 motif as defined by PFAM 32.0 as released in September 2018,
   comprising a cd03786 motif as defined by CDD v3.17 as released in September 2016,
   comprising a panther domain selected from the list comprising PTHR43174 and PTHR43174:SF3 as defined by PANTHER 17.0 as released on 23 Feb. 2022,
   that is at least 80%, at least 85%, at least 90%, at least 95%, at least 96%, at least 97%, at least 98%, at least 98.5%, or at least 99% identical to any one of the amino acid sequences as represented by SEQ ID NOs:01, 02, 03, 04, 05, 06, 07 or 08 over a stretch of at least 150, at least 160, at least 170, at least 180, at least 190, at least 200, at least 210, at least 220, at least 230, at least 240, at least 250, at least 260, at least 270, at least 280, at least 290 or at least 300 amino acid residues,
   that is at least 80%, at least 85%, at least 90%, at least 95%, at least 96%, at least 97%, at least 98%, at least 98.5%, or at least 99% identical to any one of the full-length amino acid sequences as represented by SEQ ID NOs:01, 02, 03, 04, 05, 06, 07 or 08, and/or
   as represented by any one of SEQ ID NOs:01, 02, 03, 04, 05, 06, 07 or 08.
4. Cell according to any one of previous embodiments, wherein the hydrolyzing UDP-N-acetyl-D-glucosamine-2-epimerase is a nnaA polypeptide.
5. Cell according to any one of previous embodiments, wherein the cell contains a nucleic acid molecule, which comprises a polynucleotide sequence, which encodes the hydrolyzing UDP-N-acetyl-D-glucosamine-2-epimerase.
6. Cell according to embodiment 5, wherein the nucleic acid molecule is operably linked to control sequences recognized by the cell, the nucleic acid molecule further i) being integrated in the genome of the cell and/or ii) presented to the cell on a vector.
7. Cell according to any one of embodiment 5 or 6, wherein the nucleic acid molecule is foreign to the cell.
8. Cell according to any one of previous embodiments, wherein the pathway for production of the negatively charged, preferably sialylated, oligosaccharide is a sialylation pathway, preferably the cell is genetically engineered to comprise the sialylation pathway, more preferably the cell comprises the sialylation pathway wherein the sialylation pathway has been genetically engineered.
9. Cell according to any one of previous embodiments, wherein the cell further comprises a pathway selected from the list comprising, consisting of or consisting essentially of fucosylation pathway, galactosylation pathway, N-acetylglucosaminylation pathway, N-acetylgalactosaminylation pathway, mannosylation pathway and N-acetylmannosaminylation pathway, preferably the cell is genetically engineered to comprise at least one of the pathway(s), more preferably the cell comprises at least one of the pathway(s) wherein at least one of the pathway(s) has/have been genetically engineered.
10. Cell according to any one of previous embodiments, wherein the cell:
   possesses, preferably expresses, more preferably overexpresses, one or more glycosyltransferase(s) selected from the list comprising, consisting of or consisting essentially of fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases, and/or
   is capable to produce, preferably produces, one or more nucleotide-activated sugars, preferably the cell is genetically engineered for production of one or more of the nucleotide-activated sugar(s).
11. Cell according to any one of previous embodiments, wherein the cell comprises a pathway for the synthesis of a nucleotide-activated sugar selected from the list comprising, consisting of or consisting essentially of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose, (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid, CMP-Neu5Ac, CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), CMP-2-keto-3-deoxymanno-octulonic acid (CMP-KDO), GDP-rhamnose and UDP-xylose.
12. Cell according to any one of previous embodiments, wherein the cell possesses, preferably expresses, one or more genes selected from the list comprising, consisting of or consisting essentially of mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-glutamine-D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, N-acetylglucosamine 1-phosphate uridylyltransferase, glucosamine-1-phosphate acetyltransferase, bifunctional N-acetylglucosamine-1-phosphate uridyltransferase/glucosamine-1-phosphate acetyltransferase, sialic acid synthase, N-acetylneuraminate lyase, N-acylneuraminate-9-phosphate synthase, N-acylneuraminate-9-phosphatase, sialic acid transporter, CMP kinase, CMP-sialic acid synthase, d-arabinose 5-phosphate isomerase, KDO-8P synthase, KDO 8-phosphate phosphatase, CMP-KDO synthetase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase, more preferably overexpresses one or more genes selected from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-glutamine-D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, sialic acid synthase, N-acetylneuraminate lyase, N-acylneuraminate-9-phosphate synthase, N-acylneuraminate-9-phosphatase, sialic acid transporter, CMP kinase, CMP-sialic acid synthase, d-arabinose 5-phosphate isomerase, KDO-8P synthase, KDO 8-phosphate phosphatase, CMP-KDO synthetase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase.
13. Cell according to any one of previous embodiments, wherein the cell is capable to produce, preferably produces, N-acetylmannosamine (ManNAc), a sialic acid residue and/or N-acetylglucosamine (GlcNAc), wherein the sialic acid residue is selected from the list comprising Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4; Neu4,5,7,8,9Ac5; Neu5Gc and 2-keto-3-deoxymanno-octulonic acid (KDO), preferably the cell is genetically engineered for production of ManNAc, a sialic acid residue and/or GlcNAc.
14. Cell according to any one of previous embodiments, wherein the negatively charged oligosaccharide is a sialylated oligosaccharide having at least one sialic acid residue selected from the list comprising, consisting of or consisting essentially of Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4; Neu4,5,7,8,9Ac5; Neu5Gc and 2-keto-3-deoxymanno-octulonic acid (KDO),
preferably, the negatively charged oligosaccharide is an oligosaccharide selected from the list comprising, consisting of or consisting essentially of a negatively charged, preferably sialylated, milk oligosaccharide, preferably a negatively charged, more preferably sialylated, mammalian milk oligosaccharide (MMO), more preferably a negatively charged, more preferably sialylated, human milk oligosaccharide (HMO); O-antigen; the oligosaccharide repeats present in capsular polysaccharides; peptidoglycan; an amino-sugar; Lewis-type antigen oligosaccharide; a negatively charged, preferably sialylated, animal oligosaccharide, preferably selected from the list consisting ofN-glycans and O-glycans; a negatively charged, preferably sialylated, plant oligosaccharide, preferably selected from the list consisting of N-glycans and O-glycans; sialylated oligosaccharide; N-acetyllactosamine containing negatively charged, preferably sialylated, oligosaccharide and lacto-N-biose containing negatively charged, preferably sialylated, oligosaccharide;

more preferably, the negatively charged oligosaccharide is selected from the list comprising, consisting of or consisting essentially of 3′sialyllactose (3′SL), 6′sialyllactose (6′SL), 3′-sialyllactosamine, 3,6-disialyllactose, 8,3-disialyllactose, sialylated lacto-N-triose, sialyllacto-N-tetraose a (LSTa), KDOα-2,3Galβ-1,3GlcNAcβ-1,3Galβ-1,4Glc, sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose, Neu5Ac-a2,3-Gal-b1,4-GlcNAc-b1,3-Gal, Neu5Ac-a2,3-Gal-b1,3-GlcNAc-b1,3-Gal, 3′-KDO-lactose, 3′-KDO-lactosamine, 3′-KDO-lacto-N-biose, 3′-KDO-6′sialyllactose, 3′KDO-8-sialyllactose, KDO-2,3Galβ-1,3GalNaco-1,3Gala-1,4Galβ-1,4Gal, KDOa-2,3Galβ-1,4GlcNacβ-1,3Galβ-1,4Glc, 3′-KDO-3-fucosyllactose, Neu5Ac-a2,8-Neu5Ac-a2,3-Gal-b1,3-GlcNAc-b1,3-Gal, 3′-Sialyl-2′-fucosyllactose, 6′-Sialyl-2′-fucosyllactose, 6′-Sialyl-3-fucosyllactose, Neu5Ac-a2,6-(Neu5Ac-a2,3-)Gal-b1,4-Glc, 3′-Sialyl-3-fucosyllactosamine, Fuc-a1,4-(Neu5Ac-a2,3-Gal-b1,3-)GlcNAc, 6′-Sialyllacto-N-biose, 3′-Sialyllacto-N-biose, Neu5Ac-a2,6-(GlcNAc-b1,3-)Gal-b1,4-Glc, Neu5Ac-a2,6-(Gal-b1,4-(Fuc-a1,3-)GlcNAc-b1,3-)Gal-b1,4-Glc, Neu5Ac-a2,3-Gal-b1,4-(Fuc-a1,3-)GlcNAc-b1,3-Gal-b1,4-Glc, Neu5Ac-a2,6-(Neu5Ac-a2,6-Gal-b1,4-(Fuc-a1,3-)GlcNAc-b1,3-)Gal-b1,4-Glc, Neu5Ac-a2,6-(Gal-b1,4-GlcNAc-b1,3-)Gal-b1,4-Glc, Neu5Ac-a2,6-(Gal-b1,3-GlcNAc-b1,3-)Gal-b1,4-(Fuc-a1,3-)Glc, Neu5Ac-a2,6-Gal-b1,3-GlcNAc-b1,3-Gal-b1,4-(Fuc-a1,3-)Glc, Neu5Ac-a2,3-Gal-b1,3-GlcNAc-b1,3-Gal-b1,4-(Fuc-a1,3-)Glc, Neu5Ac-a2,3-(Fuc-a1,2-)Gal-b1,3-GlcNAc-b1,3-Gal-b1,4-Glc, Neu5Ac-a2,6-(Fuc-a1,2-Gal-b1,3-GlcNAc-b1,3-)Gal-b1,4-Glc, Neu5Ac-a2,6-(Fuc-a1,2-)Gal-b1,3-GlcNAc-b1,3-Gal-b1,4-Glc, Fuc-a1,4-(Neu5Ac-a2,3-Gal-b1,3-)GlcNAc-b1,3-Gal-b1,4-Glc, Neu5Ac-a2,6-(Neu5Ac-a2,6-Gal-b1,3-GlcNAc-b1,3-)Gal-b1,4-(Fuc-a1,3-)Glc, Neu5Ac-a2,6-(Neu5Ac-a2,6-(Fuc-a1,2-)Gal-b1,3-GlcNAc-b1,3-)Gal-b1,4-Glc, Neu5Ac-a2,6-(Gal-b1,3-GlcNAc-b1,3-)Gal-b1,4-Glc, Neu5Ac-a2,6-Gal-b1,3-GlcNAc-b1,3-Gal-b1,4-Glc, Neu5Ac-a2,6-(Neu5Ac-a2,3)-Gal-b1,4-GlcNAc-b1,3-Gal-b1,4-Glc, Neu5Ac-a2,6-(Neu5Ac-a2,3-Gal-b1,3-GlcNAc-b1,3-)Gal-b1,4-Glc, Neu5Ac-a2,6-(Neu5Ac-a2,6-Gal-b1,3-GlcNAc-b1,3)-Gal-b1,4-Glc, Neu5Ac-a2,6-(Neu5Ac-a2,6-(Neu5Ac-a2,3)-Gal-b1,3-GlcNAc-b1,3)-Gal-b1,4-Glc, Neu5Ac-a2,6-(Neu5Ac-a2,3-Gal-b1,4-GlcNAc-b1,3)-Gal-b1,4-Glc, Neu5Ac-a2,6-(Neu5Ac-a2,6-Gal-b1,4-GlcNAc-b1,3)-Gal-b1,4-Glc, Neu5Ac-a2,6-(Neu5Ac-a2,6-(Neu5Ac-a2,3)-Gal-b1,4-GlcNAc-b1,3)-Gal-b1,4-Glc, Neu5Ac-a2,6-(Gal-b1,4-GlcNAc-b1,3-Gal-b1,4-GlcNAc-b1,3)-Gal-b1,4-Glc, Neu5Ac-a2,6-(Gal-b1,4-GlcNAc-b1,3)-Gal-b1,4-GlcNAc-b1,3-Gal-b1,4-Glc, Neu5Ac-a2,6-Gal-b1,4-GlcNAc-b1,3-Gal-b1,4-GlcNAc-b1,3-Gal-b1,4-Glc, Neu5Ac-a2,6-(Neu5Ac-a2,6-(Neu5Ac-a2,6-Gal-b1,4-GlcNAc-b1,3)-Gal-b1,4-GlcNAc-b1,3)-Gal-b1,4-Glc, Neu5Ac-a2,3-Gal-b1,4-GlcNAc-b1,3-Gal-b1,4-GlcNAc-b1,3-Gal-b1,4-Glc, 6′-KDO-lactose, 6′-KDO-lactosamine, 6′-KDO-lacto-N-biose, KDO-lacto-N-triose, KDO-lacto-N-tetraose, KDO-lacto-N-tetraose, and combinations thereof.
15. Cell according to any one of previous embodiments, wherein the cell is capable to produce, preferably produces, the negatively charged, preferably sialylated, oligosaccharide from one or more precursor(s), preferably the precursor is lactose.
16. Cell according to embodiment 15, wherein the cell is capable to produce, preferably produces, at least one of the one or more precursor(s), preferably the cell is capable to produce, preferably produces, all of the one or more precursor(s).
17. Cell according to any one of embodiment 15 or 16, wherein the cell is genetically engineered for the production of at least one of the one or more precursor(s), preferably the cell is genetically engineered for the production of all of the one or more precursor(s).
18. Cell according to any one of embodiments 15 to 17, wherein is capable to produce, preferably produces, lactose.
19. Cell according to any one of embodiments 15 to 18, wherein at least one of the one or more precursor(s) is internalized in the cell via one or more membrane transporter protein(s).
20. Cell according to any one of previous embodiments, wherein the cell is capable to produce and/or produces phosphoenolpyruvate (PEP).
21. Cell according to any one of previous embodiments, wherein the cell is modified for enhanced synthesis and/or supply of phosphoenolpyruvate (PEP) compared to a non-modified progenitor.
22. Cell according to any one of previous embodiments, wherein the cell is further modified for reduced degradation of acetyl-CoA and/or its main precursor pyruvate.
23. Cell according to any one of previous embodiments, wherein the cell comprises a modification for reduced production of acetate compared to a non-modified progenitor.
24. Cell according to any one of previous embodiments, wherein the cell possesses, expresses and/or overexpresses at least one gene selected from the list comprising, consisting of or consisting essentially of genes encoding a disulfide bond isomerase, a thiol oxidase or a chaperone.
25. Cell according to any one of previous embodiments, wherein the cell comprises a catabolic pathway for selected mono-, di- or oligosaccharides, which is at least partially inactivated, the mono-, di-, or oligosaccharides being involved in and/or required for the synthesis of the negatively charged, preferably sialylated, oligosaccharide.
26. Cell according to any one of previous embodiments, wherein the cell possesses, expresses and/or overexpresses a membrane transporter protein.
27. Cell according to any one of previous embodiments, wherein one or more gene(s) involved in one or more reductive pathway(s) in the cell is/are rendered less functional or is/are knocked-out.
28. Cell according to embodiment 27, wherein the one or more gene(s) involved in one or more reductive pathway(s) is/are rendered less functional by insertion, deletion and/or modification of one or more nucleotide(s) in one or more polynucleotide sequence(s) selected from the list comprising, consisting of or consisting essentially of promoter sequence, ribosome binding site, untranslated region, coding sequence and transcription terminator sequence of the one or more gene(s).
29. Cell according to any one of previous embodiments, wherein the cell is a bacterium, fungus, yeast, a plant cell, an animal cell, or a protozoan cell,
   preferably, the bacterium belongs to a phylum selected from the list comprising, consisting of or consisting essentially of Proteobacteria, Firmicutes, Cyanobacteria, Deinococcus-Thermus and Actinobacteria; more preferably, the bacterium belongs to a family selected from the list comprising, consisting of or consisting essentially of Enterobacteriaceae, Bacillaceae, Lactobacillaceae, Corynebacteriaceae and Vibrionaceae; even more preferably, the bacterium is selected from the list comprising, consisting of or consisting essentially of an Escherichia coli strain, a Bacillus subtilis strain and a Vibrio natriegens strain; even more preferably the Escherichia coli strain is a K-12 strain, most preferably the Escherichia coli K-12 strain is E. coli MG1655,
   preferably, the fungus belongs to a genus selected from the list comprising, consisting of or consisting essentially of Rhizopus, Dictyostelium, Penicillium, Mucor or Aspergillus,
   preferably, the yeast belongs to a genus selected from the list comprising, consisting of or consisting essentially of Saccharomyces, Zygosaccharomyces, Pichia, Komagataella, Hansenula, Yarrowia, Starmerella, Kluyveromyces, Debaromyces, Candida, Schizosaccharomyces, Schwanniomyces and Torulaspora; more preferably, the yeast is selected from the list comprising, consisting of or consisting essentially of Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii,
   preferably, the plant cell is an algal cell or is derived from tobacco, alfalfa, rice, tomato, cotton, rapeseed, soy, maize, or corn plant,
   preferably, the animal cell is derived from insects, amphibians, reptiles, invertebrates, fish, birds or mammalian cells excluding human embryonic stem cells, more preferably the mammalian cell is selected from the list comprising, consisting of or consisting essentially of an epithelial cell, an embryonic kidney cell, a fibroblast cell, a COS cell, a Chinese hamster ovary (CHO) cell, a murine myeloma cell, an NIH-3T3 cell, a lactocyte derived from mammalian induced pluripotent stem cells, more preferably the mammalian induced pluripotent stem cells are human induced pluripotent stem cells, a post-parturition mammary epithelium cell, a polarized mammary cell, more preferably the polarized mammary cell is selected from the list comprising, consisting of or consisting essentially of live primary mammary epithelial cells, live mammary myoepithelial cells, live mammary progenitor cells, live immortalized mammary epithelial cells, live immortalized mammary myoepithelial cells, live immortalized mammary progenitor cells, a non-mammary adult stem cell or derivatives thereof, more preferably the insect cell is derived from Spodoptera frugiperda, Bombyx mori, Mamestra brassicae, Trichoplusia ni or Drosophila melanogaster,
   preferably, the protozoan cell is a Leishmania tarentolae cell.
30. Cell according to any one of previous embodiments, wherein the cell is selected from the list consisting of prokaryotic cells and eukaryotic cells, preferably from the list consisting of yeast cells, bacterial cells, archaebacterial cells, algae cells, and fungal cells.
31. Cell according to any one of previous embodiments, wherein the cell is an E. coli or yeast with a lactose permease positive phenotype, preferably wherein the lactose permease is coded by the gene LacY or LAC12, respectively.
32. Cell according to any one of previous embodiments, wherein the cell is a viable Gram-negative bacterium that comprises a reduced or abolished synthesis of poly-N-acetylglucosamine (PNAG), Enterobacterial Common Antigen (ECA), cellulose, colanic acid, core oligosaccharides, Osmoregulated Periplasmic Glucans (OPG), glucosylglycerol, glycan and/or trehalose compared to a non-modified progenitor.
33. Cell according to any one of previous embodiments, wherein the cell produces 30 g/L or more of the negatively charged, preferably sialylated, oligosaccharide in the whole broth and/or supernatant and/or wherein the negatively charged, preferably sialylated, oligosaccharide in the whole broth and/or supernatant has a purity of at least 80% measured on the total amount of the negatively charged, preferably sialylated, oligosaccharide and its precursor(s) produced by the cell in the whole broth and/or supernatant, respectively.
34. Cell according to any one of previous embodiments, wherein the cell is stably cultured in a cultivation or incubation medium.
35. Cell according to any one of previous embodiments, wherein the cell is capable to produce and/or produces a mixture of two or more oligosaccharides wherein at least one of the two or more oligosaccharides is the negatively charged, preferably sialylated, oligosaccharide.
36. Cell according to any one of previous embodiments, wherein the cell is capable to produce and/or produces a mixture of one or more disaccharide(s) and one or more oligosaccharide(s) wherein at least one of the one or more oligosaccharide(s) is the negatively charged, preferably sialylated, oligosaccharide.
37. Cell according to any one of previous embodiments, wherein the cell is capable to produce and/or produces a mixture comprising the negatively charged, preferably sialylated, oligosaccharide and at least one non-charged (neutral) oligosaccharide.
38. Cell according to any one of previous embodiments, wherein the cell is capable to produce and/or produces a mixture comprising 1) the negatively charged, preferably sialylated, oligosaccharide and 2) a disaccharide and/or a non-charged (neutral) oligosaccharide.
39. Method for the production of a negatively charged, preferably sialylated, oligosaccharide, the method comprising:
i. cultivating and/or incubating a cell of any one of previous embodiments, in cultivation and/or incubation medium under conditions permissive to produce the negatively charged, preferably sialylated, oligosaccharide,
ii. preferably, followed by separation and/or purification of the negatively charged, preferably sialylated, oligosaccharide from the cultivation and/or incubation.
40. Method according to embodiment 39, wherein the negatively charged oligosaccharide is a sialylated oligosaccharide having at least one sialic acid residue selected from the list comprising, consisting of or consisting essentially of Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4; Neu4,5,7,8,9Ac5; Neu5Gc and 2-keto-3-deoxymanno-octulonic acid (KDO),
preferably, the negatively charged oligosaccharide is an oligosaccharide selected from the list comprising, consisting of or consisting essentially of a negatively charged, preferably sialylated, milk oligosaccharide, preferably a negatively charged, more preferably sialylated, mammalian milk oligosaccharide (MMO), more preferably a negatively charged, more preferably sialylated, human milk oligosaccharide (HMO); O-antigen; the oligosaccharide repeats present in capsular polysaccharides; peptidoglycan; an amino-sugar; Lewis-type antigen oligosaccharide; a negatively charged, preferably sialylated, animal oligosaccharide, preferably selected from the list consisting ofN-glycans and O-glycans; a negatively charged, preferably sialylated, plant oligosaccharide, preferably selected from the list consisting of N-glycans and O-glycans; sialylated oligosaccharide; N-acetyllactosamine containing negatively charged, preferably sialylated, oligosaccharide and lacto-N-biose containing negatively charged, preferably sialylated, oligosaccharide;
more preferably, the negatively charged oligosaccharide is selected from the list comprising, consisting of or consisting essentially of 3′sialyllactose (3′SL), 6′sialyllactose (6′SL), 3′-sialyllactosamine, 3,6-disialyllactose, 8,3-disialyllactose, sialylated lacto-N-triose, sialyllacto-N-tetraose a (LSTa), KDOα-2,3Galβ-1,3GlcNAcβ-1,3Galβ-1,4Glc, sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose, Neu5Ac-a2,3-Gal-b1,4-GlcNAc-b1,3-Gal, Neu5Ac-a2,3-Gal-b1,3-GlcNAc-b1,3-Gal, 3′-KDO-lactose, 3′-KDO-lactosamine, 3′-KDO-lacto-N-biose, 3′-KDO-6′sialyllactose, 3′KDO-8-sialyllactose, KDO-2,3Galβ-1,3GalNacβ-1,3Gala-1,4Galβ-1,4Gal, KDOα-2,3Galβ-1,4GlcNacβ-1,3Galβ-1,4Glc, 3′-KDO-3-fucosyllactose, Neu5Ac-a2,8-Neu5Ac-a2,3-Gal-b1,3-GlcNAc-b1,3-Gal, 3′-Sialyl-2′-fucosyllactose, 6′-Sialyl-2′-fucosyllactose, 6′-Sialyl-3-fucosyllactose, Neu5Ac-a2,6-(Neu5Ac-a2,3-)Gal-b1,4-Glc, 3′-Sialyl-3-fucosyllactosamine, Fuc-a1,4-(Neu5Ac-a2,3-Gal-b1,3-)GlcNAc, 6′-Sialyllacto-N-biose, 3′-Sialyllacto-N-biose, Neu5Ac-a2,6-(GlcNAc-b1,3-)Gal-b1,4-Glc, Neu5Ac-a2,6-(Gal-b1,4-(Fuc-a1,3-)GlcNAc-b1,3-)Gal-b1,4-Glc, Neu5Ac-a2,3-Gal-b1,4-(Fuc-a1,3-)GlcNAc-b1,3-Gal-b1,4-Glc, Neu5Ac-a2,6-(Neu5Ac-a2,6-Gal-b1,4-(Fuc-a1,3-)GlcNAc-b1,3-)Gal-b1,4-Glc, Neu5Ac-a2,6-(Gal-b1,4-GlcNAc-b1,3-)Gal-b1,4-Glc, Neu5Ac-a2,6-(Gal-b1,3-GlcNAc-b1,3-)Gal-b1,4-(Fuc-a1,3-)Glc, Neu5Ac-a2,6-Gal-b1,3-GlcNAc-b1,3-Gal-b1,4-(Fuc-a1,3-)Glc, Neu5Ac-a2,3-Gal-b1,3-GlcNAc-b1,3-Gal-b1,4-(Fuc-a1,3-)Glc, Neu5Ac-a2,3-(Fuc-a1,2-)Gal-b1,3-GlcNAc-b1,3-Gal-b1,4-Glc, Neu5Ac-a2,6-(Fuc-a1,2-Gal-b1,3-GlcNAc-b1,3-)Gal-b1,4-Glc, Neu5Ac-a2,6-(Fuc-a1,2-)Gal-b1,3-GlcNAc-b1,3-Gal-b1,4-Glc, Fuc-a1,4-(Neu5Ac-a2,3-Gal-b1,3-)GlcNAc-b1,3-Gal-b1,4-Glc, Neu5Ac-a2,6-(Neu5Ac-a2,6-Gal-b1,3-GlcNAc-b1,3-)Gal-b1,4-(Fuc-a1,3-)Glc, Neu5Ac-a2,6-(Neu5Ac-a2,6-(Fuc-a1,2-)Gal-b1,3-GlcNAc-b1,3-)Gal-b1,4-Glc, Neu5Ac-a2,6-(Gal-b1,3-GlcNAc-b1,3-)Gal-b1,4-Glc, Neu5Ac-a2,6-Gal-b1,3-GlcNAc-b1,3-Gal-b1,4-Glc, Neu5Ac-a2,6-(Neu5Ac-a2,3)-Gal-b1,4-GlcNAc-b1,3-Gal-b1,4-Glc, Neu5Ac-a2,6-(Neu5Ac-a2,3-Gal-b1,3-GlcNAc-b1,3-)Gal-b1,4-Glc, Neu5Ac-a2,6-(Neu5Ac-a2,6-Gal-b1,3-GlcNAc-b1,3)-Gal-b1,4-Glc, Neu5Ac-a2,6-(Neu5Ac-a2,6-(Neu5Ac-a2,3)-Gal-b1,3-GlcNAc-b1,3)-Gal-b1,4-Glc, Neu5Ac-a2,6-(Neu5Ac-a2,3-Gal-b1,4-GlcNAc-b1,3)-Gal-b1,4-Glc, Neu5Ac-a2,6-(Neu5Ac-a2,6-Gal-b1,4-GlcNAc-b1,3)-Gal-b1,4-Glc, Neu5Ac-a2,6-(Neu5Ac-a2,6-(Neu5Ac-a2,3)-Gal-b1,4-GlcNAc-b1,3)-Gal-b1,4-Glc, Neu5Ac-a2,6-(Gal-b1,4-GlcNAc-b1,3-Gal-b1,4-GlcNAc-b1,3)-Gal-b1,4-Glc, Neu5Ac-a2,6-(Gal-b1,4-GlcNAc-b1,3)-Gal-b1,4-GlcNAc-b1,3-Gal-b1,4-Glc, Neu5Ac-a2,6-Gal-b1,4-GlcNAc-b1,3-Gal-b1,4-GlcNAc-b1,3-Gal-b1,4-Glc, Neu5Ac-a2,6-(Neu5Ac-a2,6-(Neu5Ac-a2,6-Gal-b1,4-GlcNAc-b1,3)-Gal-b1,4-GlcNAc-b1,3)-Gal-b1,4-Glc, Neu5Ac-a2,3-Gal-b1,4-GlcNAc-b1,3-Gal-b1,4-GlcNAc-b1,3-Gal-b1,4-Glc, 6′-KDO-lactose, 6′-KDO-lactosamine, 6′-KDO-lacto-N-biose, KDO-lacto-N-triose, KDO-lacto-N-tetraose, KDO-lacto-N-tetraose, and combinations thereof.
41. Method according to any one of embodiment 39 or 40, wherein the cultivation or incubation medium comprises one or more precursor(s) that is/are used for production of the negatively charged, preferably sialylated, oligosaccharide.
42. Method according to any one of embodiments 39 to 41, wherein the cultivation medium or incubation medium contains:
   at least one carbon source selected from the list comprising, consisting of or consisting essentially of a monosaccharide; disaccharide; oligosaccharide; polysaccharide; polyol; glycerol; a complex medium including molasses, corn steep liquor, peptone, tryptone or yeast extract; glucose, glycerol, fructose, sucrose, maltose, lactose, arabinose, maltooligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate, and/or
   at least one compound selected from the list consisting of lactose, galactose, glucose, UDP-galactose (UDP-Gal), sialic acid, CMP-sialic acid, CMP-Neu5Ac and CMP-KDO.
43. Method according to any one of embodiments 39 to 42, wherein the cultivation or incubation medium:
   is a chemically defined medium, and/or
   is a minimal salt medium comprising sulphate, phosphate, chloride, ammonium, calcium, magnesium, sodium, potassium, iron, copper, zinc, manganese cobalt and/or selenium.
44. Method according to any one of embodiments 39 to 43, the method comprising at least one of the following steps:
i) Use of a cultivation or incubation medium comprising at least one precursor and/or acceptor;
ii) Adding to the cultivation or incubation medium in a reactor or incubator at least one precursor and/or acceptor feed wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of the precursor and/or acceptor feed;
iii) Adding to the cultivation or incubation medium in a reactor or incubator at least one precursor and/or acceptor feed wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than two-fold of the volume of the cultivation or incubation medium before the addition of the precursor and/or acceptor feed and wherein preferably, the pH of the precursor and/or acceptor feed is set between 2.0 and 10.0 and wherein preferably, the temperature of the precursor and/or acceptor feed is kept between 20° C. and 80° C.;
iv) Adding at least one precursor and/or acceptor feed in a continuous manner to the cultivation or incubation medium over the course of 1 day, 2 days, 3 days, 4 days, 5 days by way of a precursor and/or acceptor feeding solution;
v) Adding at least one precursor and/or acceptor feed in a continuous manner to the cultivation or incubation medium over the course of 1 day, 2 days, 3 days, 4 days, 5 days by way of a precursor and/or acceptor feeding solution and wherein the concentration of the precursor and/or acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably, the pH of the precursor and/or acceptor feeding solution is set between 2.0 and 10.0 and wherein preferably, the temperature of the precursor and/or acceptor feeding solution is kept between 20° C. and 80° C.;
said method resulting in the negatively charged, preferably sialylated, oligosaccharide, with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation,
wherein preferably the precursor is selected from the list comprising sialic acid, CMP-sialic acid, CMP-Neu5Ac, CMP-KDO, glucose, galactose and UDP-galactose, and wherein preferably the acceptor is lactose.
45. Method according to any one of embodiments 39 to 44, the method comprising at least one of the following steps:
i) Use of a cultivation or incubation medium comprising at least 50, more preferably at least 75, more preferably at least 100, more preferably at least 120, more preferably at least 150 grams of precursor per liter of initial reactor or incubator volume wherein the reactor or incubator volume ranges from 250 mL to 10.000 m³ (cubic meter);
ii) Use of a cultivation or incubation medium comprising at least 50, more preferably at least 75, more preferably at least 100, more preferably at least 120, more preferably at least 150 grams of acceptor per liter of initial reactor or incubator volume wherein the reactor or incubator volume ranges from 250 mL to 10.000 m³ (cubic meter);
iii) Adding to the cultivation or incubation medium in a reactor or incubator a precursor feed comprising at least 50, more preferably at least 75, more preferably at least 100, more preferably at least 120, more preferably at least 150 grams of precursor per liter of initial reactor or incubator volume wherein the reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of the precursor feed;
iv) Adding to the cultivation or incubation medium in a reactor or incubator an acceptor feed comprising at least 50, more preferably at least 75, more preferably at least 100, more preferably at least 120, more preferably at least 150 grams of acceptor per liter of initial reactor or incubator volume wherein the reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of the acceptor feed;
v) Adding to the cultivation or incubation medium a precursor feed comprising at least 50, more preferably at least 75, more preferably at least 100, more preferably at least 120, more preferably at least 150 grams of precursor per liter of initial reactor or incubator volume wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of the precursor feed and wherein preferably, the pH of the precursor feed is set between 2.0 and 10.0 and wherein preferably, the temperature of the precursor feed is kept between 20° C. and 80° C.;
vi) Adding to the cultivation or incubation medium an acceptor feed comprising at least 50, more preferably at least 75, more preferably at least 100, more preferably at least 120, more preferably at least 150 grams of acceptor per liter of initial reactor or incubator volume wherein the total reactor or incubator volume ranges from 250 mL to 10.000 m³ (cubic meter), preferably in a continuous manner, and preferably so that the final volume of the cultivation or incubation medium is not more than three-fold, preferably not more than two-fold, more preferably less than 2-fold of the volume of the cultivation or incubation medium before the addition of the acceptor feed and wherein preferably, the pH of the acceptor feed is set between 2.0 and 10.0 and wherein preferably, the temperature of the acceptor feed is kept between 20° C. and 80° C.;
vii) Adding a precursor and/or acceptor feed in a continuous manner to the cultivation or incubation medium over the course of 1 day, 2 days, 3 days, 4 days, 5 days by way of a precursor and/or acceptor feeding solution;
viii) Adding a precursor feed in a continuous manner to the cultivation or incubation medium over the course of 1 day, 2 days, 3 days, 4 days, 5 days by way of a precursor feeding solution and wherein the concentration of the precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the precursor feeding solution is set between 2.0 and 10.0 and wherein preferably, the temperature of the precursor feeding solution is kept between 20° C. and 80° C.;
ix) Adding an acceptor feed in a continuous manner to the cultivation or incubation medium over the course of 1 day, 2 days, 3 days, 4 days, 5 days by way of an acceptor feeding solution and wherein the concentration of the acceptor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the acceptor feeding solution is set between 2.0 and 10.0 and wherein preferably, the temperature of the acceptor feeding solution is kept between 20° C. and 80° C.;
said method resulting in the negatively charged, preferably sialylated, oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of the cultivation or incubation,
wherein preferably the precursor is selected from the list comprising sialic acid, CMP-sialic acid, CMP-Neu5Ac, CMP-KDO, glucose, galactose and UDP-galactose, and wherein preferably the acceptor is lactose.
46. Method according to any one of embodiments 39 to 45, wherein the cell produces 30 g/L or more of the negatively charged, preferably sialylated, oligosaccharide in the whole broth and/or supernatant and/or wherein the negatively charged, preferably sialylated, oligosaccharide in the whole broth and/or supernatant has a purity of at least 80% measured on the total amount of negatively charged, preferably sialylated, oligosaccharide and its precursor(s) produced by the cell in the whole broth and/or supernatant, respectively.
47. Method according to any one of embodiments 39 to 46, wherein the cell produces a mixture of two or more oligosaccharides wherein at least one of the two or more oligosaccharides is the negatively charged, preferably sialylated, oligosaccharide.
48. Method according to any one of embodiments 39 to 47, wherein the cell produces a mixture of one or more disaccharide(s) and one or more oligosaccharide(s) wherein at least one of the one or more oligosaccharide(s) is the negatively charged, preferably sialylated, oligosaccharide.
49. Method according to any one of embodiments 39 to 48, wherein the cell produces a mixture comprising the negatively charged, preferably sialylated, oligosaccharide and at least one non-charged (neutral) oligosaccharide.
50. Method according to any one of embodiments 39 to 49, wherein the cell produces a mixture comprising 1) the negatively charged, preferably sialylated, oligosaccharide and 2) a disaccharide and/or a non-charged (neutral) oligosaccharide.
51. Method according to any one of embodiments 39 to 50, wherein the negatively charged, preferably sialylated, oligosaccharide is recovered from the cultivation or incubation medium and/or the cell, more preferably the negatively charged, preferably sialylated, oligosaccharide is purified.
52. Method according to any one of embodiments 39 to 51, wherein the separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, affinity chromatography, ion exchange chromatography, hydrophobic interaction chromatography and/or gel filtration, ligand exchange chromatography, electrodialysis.
53. Method according to any one of embodiments 39 to 52, wherein the purification comprises at least one of the following steps: use of activated charcoal or carbon, use of charcoal, nanofiltration, ultrafiltration, electrophoresis, enzymatic treatment or ion exchange, temperature adjustment, pH adjustment, pH adjustment with an alkaline or acidic solution, use of alcohols, use of aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.
54. Use of a cell according to any one of embodiments 1 to 38 for the production of a negatively charged, preferably sialylated, oligosaccharide.
55. Use of a method according to any one of embodiments 39 to 53 for the production of a negatively charged, preferably sialylated, oligosaccharide.

This disclosure will be described in more detail in the examples. The following examples will serve as further illustration and clarification of the disclosure and are not intended to be limiting.

### EXAMPLES

### Example 1. Materials and Methods

A. Escherichia coli

### Media and Cultivation

The Luria Broth (LB) medium consisted of 1% tryptone peptone (Difco, Erembodegem, Belgium), 0.5% yeast extract (Difco) and 0.5% sodium chloride (VWR. Leuven, Belgium). The minimal medium used in cultivation experiments in 96-well plates or in shake flasks contained 2.00 g/L NH₄Cl, 5.00 g/L (NH₄)₂SO₄, 2.993 g/L KH₂PO₄, 7.315 g/L K₂HPO₄, 8.372 g/L MOPS, 0.5 g/L NaCl, 0.5 g/L MgSO₄·7H₂O, 30 g/L sucrose or 30 g/L glycerol, 1 ml/L vitamin solution, 100 μl/L molybdate solution, and 1 mL/L selenium solution. As precursor(s) and/or acceptor(s) for saccharide synthesis, compounds like e.g., galactose, glucose, fructose, fucose, lactose, LacNAc, LNB, a co-factor and sialic acid could be added to the medium. The minimal medium was set to a pH of 7 with 1M KOH. Vitamin solution consisted of 3.6 g/L FeCl₂·4H₂O, 5.0 g/L CaCl₂·2H₂O, 1.3 g/L MnCl₂·2H₂O, 0.38 g/L CuCl₂·2H₂O, 0.5 g/L CoCl₂·6H₂O, 0.94 g/L ZnCl₂, 0.0311 g/L H₃BO₄, 0.4 g/L Na₂EDTA·2H₂O and 1.01 g/L thiamine·HCl. The molybdate solution contained 0.967 g/L NaMoO₄·2H₂O. The selenium solution contained 42 g/L Seo2. The minimal medium for fermentations contained 6.75 g/L NH₄Cl, 1.25 g/L (NH₄)₂SO₄, 2.93 g/L KH₂PO₄ and 7.31 g/L KH₂PO₄, 0.5 g/L NaCl, 0.5 g/L MgSO₄·7H₂O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid, 0.30 g/L GlcNAc, 20 g/L lactose, 20 g/L LacNAc, 20 g/L LNB, 20 g/L LN3, 20 g/L LNT and/or 20 g/L LNnT were additionally added to the medium. Complex medium was sterilized by autoclaving (121° C., 21 min) and minimal medium by filtration (0.22 μm Sartorius). When necessary, the medium was made selective by adding an antibiotic: e.g., chloramphenicol (20 mg/L), carbenicillin (100 mg/L), spectinomycin (40 mg/L) and/or kanamycin (50 mg/L).

A preculture of 96-well microtiter plate experiments was started from a cryovial, in 150 μL LB and was incubated overnight at 37° C. on an orbital shaker at 800 rpm. This culture was used as inoculum for a 96well square microtiter plate, with 400 μL minimal medium by diluting 400×. These final 96-well culture plates were then incubated at 37° C. on an orbital shaker at 800 rpm for 72 h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations).

Also, a dilution of the cultures was made to measure the optical density at 600 nm. The cell performance index or CPI was determined by dividing the oligosaccharide concentrations by the biomass, in relative percentages compared to a reference strain. The biomass is empirically determined to be approximately ⅓rd of the optical density measured at 600 nm.

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H₂SO₄ and 20% NH₄OH. The exhaust gas was cooled. 10% solution of silicone antifoaming agent was added when foaming raised during the fermentation.

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). The pET28b(+) vector was obtained from Millipore and adapted for Golden Gate cloning. Plasmids were maintained in the host E. coli DH5alpha (F⁻, phi80dlacZΔM15, Δ(lacZYA-argF) U169, deoR, recA1, endA1, hsdR17(rk⁻, mk⁺), phoA, supE44, lambda⁻, thi-1, gyrA96, relA1) bought from Invitrogen.

### Strains and Mutations

Escherichia coli K12 MG1655 [λ-, F-, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). All constitutive promoters, UTRs and terminator sequences originated from the libraries described by Cambray et al. (Nucleic Acids Res. 2013, 41(9), 5139-5148), Dunn et al. (Nucleic Acids Res. 1980, 8, 2119-2132), Edens et al. (Nucleic Acids Res. 1975, 2, 1811-1820), Kim and Lee (FEBS Letters 1997, 407, 353-356) and Mutalik et al. (Nat. Methods 2013, No. 10, 354-360). Genes were ordered synthetically at Twist Bioscience (twistbioscience.com) or IDT (eu.idtdna.com) and the codon usage was adapted using the tools of the supplier. All strains were stored in cryovials at −80° C. (overnight LB culture mixed in a 1:1 ratio with 70% glycerol).

In an example for GDP-fucose production, the mutant strain was derived from E. coli K12 MG1655 comprising a knock-out of the E. coli wcaJ gene and genomic knock-ins of constitutive transcriptional units containing a sucrose transporter like e.g., CscB from E. coli W (UniProt ID E0IXR1), a fructose kinase like e.g., Frk originating from Zymomonas mobilis (UniProt ID Q03417) and a sucrose phosphorylase like e.g., BaSP originating from Bifidobacterium adolescentis (UniProt ID A0ZZH6). GDP-fucose production can further be optimized in the mutant E. coli strain by genomic knock-outs of any one or more of the E. coli genes comprising glgC, agp, pfkA, pfkB, pgi, arcA, iclR, pgi and ion as described in WO2016075243 and WO2012007481. GDP-fucose production can additionally be optimized comprising genomic knock-ins of constitutive transcriptional units for a mannose-6-phosphate isomerase like e.g., manA from E. coli (UniProt ID P00946), a phosphomannomutase like e.g., manB from E. coli (UniProt ID P24175), a mannose-1-phosphate guanylyltransferase like e.g., manC from E. coli (UniProt ID P24174), a GDP-mannose 4,6-dehydratase like e.g., gmd from E. coli (UniProt ID P0AC88) and a GDP-L-fucose synthase like e.g., fcl from E. coli (UniProt ID P32055). GDP-fucose production can also be obtained by genomic knock-outs of the E. coli fucK and fucI genes and genomic knock-ins of constitutive transcriptional units containing a fucose permease like e.g., fucP from E. coli (UniProt ID P11551) and a bifunctional enzyme with fucose kinase/fucose-1-phosphate guanylyltransferase activity like e.g., fkp from Bacteroides fragilis (UniProt ID SUV40286.1). All mutant strains can be additionally modified with genomic knock-outs of the E. coli LacZ, LacY and LacA genes and with a genomic knock-in of a constitutive transcriptional unit for a lactose permease like e.g., the E. coli LacY (UniProt ID P02920). For production of a fucosylated saccharide, the mutant GDP-fucose production strain was additionally modified with an expression plasmid comprising a constitutive transcriptional unit for a fucosyltransferase, like e.g., the alpha-1,2-fucosyltransferase HpFutC from H. pylori (UniProt ID Q9X435) to produce 2′-fucosyllactose (2′FL) or the alpha-1,3-fucosyltransferase HpFucT from H. pylori (UniProt ID 030511) to produce 3-fucosyllactose (3-FL), or both the alpha-1,2-fucosyltransferase HpFutC from H. pylori (UniProt ID Q9X435) and the alpha-1,3-fucosyltransferase HpFucT from H. pylori (UniProt ID 030511) to produce difucosyllactose (DiFL).

In an example to produce lacto-N-triose (LN3, GlcNAc-β1,3-Gal-β1,4-Glc), the mutant strain was derived from E. coli K12 MG1655 and modified with a knock-out of the E. coli lacZ, lacY, lacA and nagB genes and with genomic knock-ins of constitutive transcriptional units for a lactose permease like e.g., the E. coli LacY (UniProt ID P02920) and a galactoside beta-1,3-N-acetylglucosaminyltransferase like e.g., lgtA (UniProt ID Q9JXQ6) from N. meningitidis. In an example for production of LN3 derived oligosaccharides like lacto-N-tetraose (LNT, Gal-β1,3-GlcNAc-β1,3-Gal-β1,4-Glc), the mutant LN3 producing strain was further modified with a constitutive transcriptional unit delivered to the strain either via genomic knock-in or from an expression plasmid for an N-acetylglucosamine beta-1,3-galactosyltransferase like e.g., wbgO (Uniprot ID D3QY14) from E. coli O55:H7. In an example for production of LN3 derived oligosaccharides like lacto-N-neotetraose (LNnT, Gal-β1,4-GlcNAc-β1,3-Gal-β1,4-Glc), the mutant LN3 producing strain was further modified with a constitutive transcriptional unit delivered to the strain either via genomic knock-in or from an expression plasmid for an N-acetylglucosamine beta-1,4-galactosyltransferase like e.g., LgtB (Uniprot ID Q51116, sequence version 02, 1 Dec. 2000) from Neisseria meningitidis. LN3, LNT and/or LNnT production can further be optimized in the mutant E. coli strains with genomic knock-outs of the E. coli genes comprising any one or more of galT, ushA, ldhA and agp. The mutant LN3, LNT and/or LNnT producing strains can also be optionally modified for enhanced UDP-GlcNAc production with a genomic knock-in of a constitutive transcriptional unit for an L-glutamine-D-fructose-6-phosphate aminotransferase like e.g., the E. coli glmS (UniProt ID P17169, sequence version 04, 23 Jan. 2007) or a mutant glmS*54 from E. coli with SEQ ID NO:09 and differing from the wild-type E. coli glmS, having UniProt ID P17169, by an A39T, an R250C and an G472S mutation as described by Deng et al. (Biochimie 88, 419-29 (2006). The mutant E. coli strains can also optionally be adapted with a genomic knock-in of a constitutive transcriptional unit for an UDP-glucose-4-epimerase like e.g., galE from E. coli (UniProt ID P09147), a phosphoglucosamine mutase like e.g., glmM from E. coli (UniProt ID P31120, sequence version 03, 23 Jan. 2007) and an N-acetylglucosamine-1-phosphate uridylyltransferase/glucosamine-1-phosphate acetyltransferase like e.g., glmU from E. coli (UniProt ID P0ACC7). The mutant LN3, LNT and/or LNnT producing E. coli strains can also optionally be adapted for growth on sucrose via genomic knock-ins of constitutive transcriptional units containing a sucrose transporter like e.g., CscB from E. coli W (UniProt ID E0IXR1), a fructose kinase like e.g., Frk originating from Zymomonas mobilis (UniProt ID Q03417) and a sucrose phosphorylase like e.g., BaSP originating from Bifidobacterium adolescentis (UniProt ID A0ZZH6).

In an example for sialic acid production, the mutant strain was derived from E. coli K12 MG1655 comprising genomic knock-ins of constitutive transcriptional units containing a hydrolyzing UDP-N-acetylglucosamine 2-epimerase like e.g., NeuC from C. jejuni (UniProt ID Q93MP8) or any one of SEQ ID NO:01, 02, 03, 04, 05, 06, 07 or 08 and an N-acetylneuraminate synthase like e.g., NeuB from N. meningitidis (UniProt ID E0NCD4).

Alternatively, and/or additionally, sialic acid production can be obtained by genomic knock-ins of constitutive transcriptional units containing one or more copies of a glucosamine 6-phosphate N-acetyltransferase like e.g., GNA1 from Saccharomyces cerevisiae (UniProt ID P43577), an N-acetylglucosamine 2-epimerase like e.g., AGE from Bacteroides ovatus (UniProt ID A7LVG6) and an N-acetylneuraminate synthase like e.g., NeuB from Neisseria meningitidis (UniProt ID E0NCD4).

Alternatively and/or additionally, sialic acid production can be obtained by genomic knock-ins of constitutive transcriptional units containing a phosphoglucosamine mutase like e.g., glmM from E. coli (UniProt ID P31120, sequence version 03, 23 Jan. 2007), an N-acetylglucosamine-1-phosphate uridyltransferase/glucosamine-1-phosphate acetyltransferase like e.g., glmU from E. coli (UniProt ID P0ACC7), a hydrolyzing UDP-N-acetylglucosamine 2-epimerase like e.g., NeuC from C. jejuni (UniProt ID Q93MP8) or any one of SEQ ID NO:01, 02, 03, 04, 05, 06, 07 or 08 and an N-acetylneuraminate synthase like e.g., NeuB from N. meningitidis (UniProt ID E0NCD4).

Alternatively, and/or additionally, sialic acid production can be obtained by genomic knock-ins of constitutive transcriptional units containing a bifunctional UDP-GlcNAc 2-epimerase/N-acetylmannosamine kinase like e.g., from Mus musculus (strain C57BL/6J) (UniProt ID Q91WG8), an N-acylneuraminate-9-phosphate synthetase like e.g., from Pseudomonas sp. UW4 (UniProt ID K9NPH9) and an N-acylneuraminate-9-phosphatase like e.g., from Bacteroides thetaiotaomicron (UniProt ID Q8A712).

Sialic acid production can further be optimized in the mutant E. coli strain with genomic knock-outs of the E. coli genes comprising any one or more of nagA, nagB, nagC, nagD, nagE, nanA, nanE, nanK, manX, manY and manZ as described in WO18122225, and/or genomic knock-outs of the E. coli genes comprising any one or more of nanT, poxB, ldhA, adhE, aldB, pflA, pflC, ybiY, ackA and/or pta and with genomic knock-ins of constitutive transcriptional units comprising one or more copies of an L-glutamine-D-fructose-6-phosphate aminotransferase like e.g., E. coli glmS (UniProt ID P17169, sequence version 04, 23 Jan. 2007) or a mutant glmS*54 from E. coli with SEQ ID NO:09 and differing from the wild-type E. coli glmS, having UniProt ID P17169, by an A39T, an R250C and an G472S mutation as described by Deng et al. (Biochimie 88, 419-29 (2006), preferably a phosphatase like any one of e.g., the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae or BsAraL from Bacillus subtilis as described in WO18122225 and an acetyl-CoA synthetase like e.g., acs from E. coli (UniProt ID P27550).

For sialylated oligosaccharide production, the sialic acid production strains were further modified to express an N-acylneuraminate cytidylyltransferase like e.g., the NeuA enzyme from P. multocida (UniProt ID A0A849CI62) and to express a sialyltransferase like e.g., the alpha-2,3-siayltransferase PmultST3 from P. multocida (UniProt ID Q9CLP3) or a PmultST3-like polypeptide (SEQ ID NO: 10) consisting of amino acid residues 1 to 268 of UniProt ID Q9CLP3 having beta-galactoside alpha-2,3-sialyltransferase activity or the alpha-2,6-sialyltransferase PdST6 from P. damselae (UniProt ID O66375) or a PdST6-like polypeptide (SEQ ID NO: 11) consisting of amino acid residues 108 to 497 of UniProt ID O66375 having beta-galactoside alpha-2,6-sialyltransferase activity. Constitutive transcriptional units of the N-acylneuraminate cytidylyltransferase and the sialyltransferase can be delivered to the mutant strain either via genomic knock-in or via expression plasmids. If the mutant strains producing sialic acid and CMP-sialic acid were intended to make sialylated lactose structures, the strains were additionally modified with genomic knock-outs of the E. coli LacZ, LacY and LacA genes and with a genomic knock-in of a constitutive transcriptional unit for a lactose permease like e.g., E. coli LacY (UniProt ID P02920). All mutant strains producing sialic acid, CMP-sialic acid and/or sialylated saccharides could optionally be adapted for growth on sucrose via genomic knock-ins of constitutive transcriptional units containing a sucrose transporter like e.g., CscB from E. coli W (UniProt ID E0IXR1), a fructose kinase like e.g., Frk originating from Z. mobilis (UniProt ID Q03417) and a sucrose phosphorylase like e.g., BaSP from B. adolescentis (UniProt ID A0ZZH6).

In another example, the mutant E. coli strains adapted for LNT production as described herein can also be further modified with a hydrolyzing UDP-N-acetylglucosamine 2-epimerase like e.g., NeuC from C. jejuni (UniProt ID Q93MP8) or any one of SEQ ID NO:01, 02, 03, 04, 05, 06, 07 or 08 and an N-acetylneuraminate synthase like e.g., NeuB from N. meningitidis (UniProt ID E0NCD4) and an expression plasmid comprising containing constitutive expression cassettes for the N-acylneuraminate cytidylyltransferase (NeuA) from P. multocida (UniProt ID A0A849CI62) and 1) the alpha-2,3-sialyltransferase PmultST3 from P. multocida (UniProt ID Q9CLP3) or a PmultST3-like polypeptide (SEQ ID NO: 10) consisting of amino acid residues 1 to 268 of UniProt ID Q9CLP3 having beta-galactoside alpha-2,3-sialyltransferase activity or 2) the alpha-2,6-sialyltransferase (PdST6) from Photobacterium damselae (UniProt ID 066375) or a PdST6-like polypeptide (SEQ ID NO: 11) consisting of amino acid residues 108 to 497 of UniProt ID 066375 having beta-galactoside alpha-2,6-sialyltransferase activity to produce 1) LSTa (Neu5Ac-α2,3-Gal-β1,3-GlcNAc-β1,3-Gal-β1,4-Glc) or 2) LSTb (Gal-β1,3-(Neu5Ac-α2,6)-GlcNAc-β1,3-Gal-β1,4-Glc), respectively. In another example, the mutant E. coli strains adapted for LNnT production as described herein can also be further modified with a hydrolyzing UDP-N-acetylglucosamine 2-epimerase like e.g., NeuC from C. jejuni (UniProt ID Q93MP8) or any one of SEQ ID NO:01, 02, 03, 04, 05, 06, 07 or 08 and an N-acetylneuraminate synthase like e.g., NeuB from N. meningitidis (UniProt ID E0NCD4) and an expression plasmid comprising containing constitutive expression cassettes for the N-acylneuraminate cytidylyltransferase (NeuA) from P. multocida (UniProt ID A0A849CI62) and 1) the alpha-2,3-sialyltransferase PmultST3 from P. multocida (UniProt ID Q9CLP3) or a PmultST3-like polypeptide (SEQ ID NO: 10) consisting of amino acid residues 1 to 268 of UniProt ID Q9CLP3 having beta-galactoside alpha-2,3-sialyltransferase activity or 2) the alpha-2,6-sialyltransferase (PdST6) from P. damselae (UniProt ID 066375) or a PdST6-like polypeptide (SEQ ID NO: 11) consisting of amino acid residues 108 to 497 of UniProt ID 066375 having beta-galactoside alpha-2,6-sialyltransferase activity to produce 1) LSTd (Neu5Ac-α2,3-Gal-β1,4-GlcNAc-β1,3-Gal-β1,4-Glc) or 2) LSTc (Neu5Ac-α2,6-Gal-β1,4-GlcNAc-β1,3-Gal-β1,4-Glc), respectively.

Preferably but not necessarily, any one or more of the glycosyltransferases, the proteins involved in nucleotide-activated sugar synthesis and/or the membrane transporter proteins were N- and/or C-terminally fused to a solubility enhancer tag like e.g., a SUMO-tag, an MBP-tag, His, FLAG, Strep-II, Halo-tag, NusA, thioredoxin, GST and/or the Fh8-tag to enhance their solubility (Costa et al., Front. Microbiol. 2014, https://doi.org/10.3389/fnicb.2014.00063; Fox et al., Protein Sci. 2001, 10(3), 622-630; Jia and Jeaon, Open Biol. 2016, 6: 160196).

Optionally, the modified E. coli strains were modified with a genomic knock-in of a constitutive transcriptional unit encoding a chaperone protein like e.g., DnaK, DnaJ, GrpE or the GroEL/ES chaperonin system (Baneyx F., Palumbo J. L. (2003) Improving Heterologous Protein Folding via Molecular Chaperone and Foldase Co-Expression. In: Vaillancourt P. E. (eds) E. coli Gene Expression Protocols. Methods in Molecular Biology™, vol 205. Humana Press).

Optionally, the mutant E. coli strains are modified to create a glycominimized E. coli strain comprising genomic knock-out of any one or more of non-essential glycosyltransferase genes comprising pgaC, pgaD, rfe, rffT, rffM, bcsA, bcsB, bcsC, wcaA, wcaC, wcaE, weal, wcaJ, wcaL, waaH, waaF, waaC, waaU, waaZ, waaJ, waaO, waaB, waaS, waaG, waaQ, wbbl, arnC, arnT, yfdH, wbbK, opgG, opgH, ycjM, glgA, glgB, malQ, otsA and yaiP.

Optionally, wild-type E. coli K12 MG1655 cells or mutant E. coli K12 MG1655 cells modified for HMO synthesis were modified for protein production by mutation in or genomic knock-out of one or more cytoplasmic reductive pathway genes like e.g., glutathione reductase (gor) and/or thioredoxin reductase (trxB) rendering the genes less functional or eliminated compared to non-modified cells. Optionally, the mutant cells were further modified with genomic knock-ins of constitutive transcriptional units containing one or more copies of a disulfide bond isomerase, a thiol oxidase and/or a chaperone like e.g., dsbC from E. coli (UniProt ID P0AEG6), PDI from S. cerevisiae (UniProt ID P17967, Sequence version 02 (2 Jun. 2021)), QSOX from Homo sapiens (UniProt ID 000391, Sequence version 03 (2 Jun. 2021)). Alternatively, and/or additionally, the mutant cells were further modified with genomic knock-outs of one or more gene(s) encoding a protease like e.g., lon, OmpT and/or a nucleotide-sugar degrading enzyme like e.g., ushA.

B. Saccharomyces cerevisiae

### Media and Cultivation

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals). As precursor(s) and/or acceptor(s) for saccharide synthesis, compounds like e.g., galactose, glucose, fructose, fucose, lactose, LacNAc, LNB, a co-factor and sialic acid could be added to the medium. In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm.

Also, a dilution of the cultures was made to measure the optical density at 600 nm. The cell performance index or CPI was determined by dividing the oligosaccharide concentrations by the biomass, in relative percentages compared to a reference strain. The biomass is empirically determined to be approximately ⅓rd of the optical density measured at 600 nm.

### Strains, Plasmids and Mutations

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995). Genes were expressed using synthetic constitutive promoters, as described by e.g., Blazeck (Biotechnology and Bioengineering, Vol. 109, No. 11, 2012), Redden and Alper (Nat. Commun. 2015, 6, 7810), Liu et al. (Microb. Cell Fact. 2020, 19, 38), Xu et al. (Microb. Cell Fact. 2021, 20, 148) and Lee et al. (ACS Synth. Biol. 2015, 4(9), 975-986).

In an example to produce GDP-fucose, a yeast expression plasmid like p2a_2μ_Fuc (Chan 2013, Plasmid 70, 2-17) can be used for expression of foreign genes in S. cerevisiae. This plasmid contains an ampicillin resistance gene and a bacterial origin of replication to allow for selection and maintenance in E. coli and the 2p yeast on and the Ura3 selection marker for selection and maintenance in yeast. This plasmid is further modified with constitutive transcriptional units for a lactose permease like e.g., LAC12 from K. lactis (UniProt ID P07921), a GDP-mannose 4,6-dehydratase like e.g., gmd from E. coli (UniProt ID P0AC88) and a GDP-L-fucose synthase like e.g., fcl from E. coli (UniProt ID P32055). The yeast expression plasmid p2a_2μ_Fuc2 can be used as an alternative expression plasmid of the p2a_2μ_Fuc plasmid comprising next to the ampicillin resistance gene, the bacterial ori, the 2μ yeast on and the Ura3 selection marker constitutive transcriptional units for a lactose permease like e.g., LAC12 from K. lactis (UniProt ID P07921), a fucose permease like e.g., fucP from E. coli (UniProt ID P11551) and a bifunctional enzyme with fucose kinase/fucose-1-phosphate guanylyltransferase activity like e.g., fkp from Bacteroides fragilis (UniProt ID SUV40286.1). To further produce a fucosylated saccharide, the p2a_2μ_Fuc and its variant the p2a_2μ_Fuc2, additionally contained a constitutive transcriptional unit for a fucosyltransferase.

In an example to produce UDP-galactose, a yeast expression plasmid can be derived from the pRS420-plasmid series (Christianson et al., 1992, Gene 110: 119-122) containing the HIS3 selection marker and a constitutive transcriptional unit for an UDP-glucose-4-epimerase like e.g., galE from E. coli (UniProt ID P09147). This plasmid can be further modified with constitutive transcriptional units for a lactose permease like e.g., LAC12 from K. lactis (UniProt ID P07921) and a galactoside beta-1,3-N-acetylglucosaminyltransferase activity like e.g., lgtA from N. meningitidis (UniProt ID Q9JXQ6) to produce LN3. In an example to further produce LN3-derived oligosaccharides like LNT, the mutant LN3 producing strains were further modified with a constitutive transcriptional unit for an N-acetylglucosamine beta-1,3-galactosyltransferase like e.g., WbgO (Uniprot ID D3QY14) from E. coli O55:H7. In an example for production of LN3 derived oligosaccharides like lacto-N-neotetraose (LNnT, Gal-β1,4-GlcNAc-β1,3-Gal-β1,4-Glc), the mutant LN3 producing strain were further modified with a constitutive transcriptional unit for an N-acetylglucosamine beta-1,4-galactosyltransferase like e.g., LgtB (Uniprot ID Q51116, sequence version 02, 1 Dec. 2000) from N. meningitidis.

In an example to produce sialic acid and CMP-sialic acid, a yeast expression plasmid was derived from the pRS420-plasmid series (Christianson et al., 1992, Gene 110: 119-122) containing the TRP1 selection marker and constitutive transcriptional units for an L-glutamine-D-fructose-6-phosphate aminotransferase like e.g., E. coli glmS (UniProt ID P17169 (sequence version 04 (23 Jan. 2007)) or a mutant glmS*54 from E. coli with SEQ ID NO:09 and differing from the wild-type E. coli glmS, having UniProt ID P17169, by an A39T, an R250C and an G472S mutation as described by Deng et al. (Biochimie 88, 419-29 (2006), a phosphatase like e.g., SurE from E. coli (UniProt ID P0A840), an N-acylglucosamine 2-epimerase like e.g., AGE from B. ovatus (UniProt ID A7LVG6), an N-acetylneuraminate synthase like e.g., NeuB from N. meningitidis (UniProt ID E0NCD4) and an N-acylneuraminate cytidylyltransferase like e.g., NeuA from P. multocida (UniProt A0A849CI62). Optionally, a constitutive transcriptional unit for a glucosamine 6-phosphate N-acetyltransferase like e.g., GNA1 from S. cerevisiae (UniProt ID P43577) was added as well. In another example to produce sialic acid and CMP-sialic acid, a yeast expression plasmid was derived from the pRS420-plasmid series (Christianson et al., 1992, Gene 110: 119-122) containing the TRP1 selection marker and constitutive transcriptional units for an L-glutamine-D-fructose-6-phosphate aminotransferase like e.g., E. coli glmS (UniProt ID P17169 (sequence version 04 (23 Jan. 2007)) or a mutant glmS*54 from E. coli with SEQ ID NO:09, a hydrolyzing UDP-N-acetylglucosamine 2-epimerase like e.g., NeuC from C. jejuni (UniProt ID Q93MP8) or any one of SEQ ID NO:01, 02, 03, 04, 05, 06, 07 or 08, an N-acetylneuraminate synthase like e.g., NeuB from N. meningitidis (UniProt ID E0NCD4) and an N-acylneuraminate cytidylyltransferase like e.g., NeuA from P. multocida (UniProt A0A849CI62).

Optionally, a constitutive transcriptional unit for a siderophore transporter like e.g., entS from E. coli (UniProt ID P24077, sequence version 02 (1 Nov. 1997)) was added as well. In an example to produce a sialylated saccharide, the plasmid further comprised constitutive transcriptional units for a lactose permease like e.g., LAC12 from K. lactis (UniProt ID P07921), and a sialyltransferase like e.g., an alpha-2,3-sialyltransferase like e.g., the alpha-2,3-sialyltransferase PmultST3 from P. multocida (UniProt ID Q9CLP3) or a PmultST3-like polypeptide (SEQ ID NO:10) consisting of amino acid residues 1 to 268 of UniProt ID Q9CLP3 having beta-galactoside alpha-2,3-sialyltransferase activity or an alpha-2,6-sialyltransferase like e.g., the alpha-2,6-sialyltransferase (PdST6) from P. damselae (UniProt ID 066375) or a PdST6-like polypeptide (SEQ ID NO: 11) consisting of amino acid residues 108 to 497 of UniProt ID 066375 having beta-galactoside alpha-2,6-sialyltransferase activity.

Preferably but not necessarily, any one or more of the glycosyltransferases and/or the proteins involved in nucleotide-activated sugar synthesis were N- and/or C-terminally fused to a SUMOstar tag (e.g., obtained from pYSUMOstar, Life Sensors, Malvern, PA) to enhance their solubility.

Optionally, the mutant yeast strains were modified with a genomic knock-in of a constitutive transcriptional unit encoding a chaperone protein like e.g., Hsp31, Hsp32, Hsp33, Sno4, Kar2, Ssb1, Sse1, Sse2, Ssa1, Ssa2, Ssa3, Ssa4, Ssb2, Ecm10, Ssc1, Ssq1, Ssz1, Lhs1, Hsp82, Hsc82, Hsp78, Hsp104, Tcp1, Cct4, Cct8, Cct2, Cct3, Cct5, Cct6 or Cct7 (Gong et al., 2009, Mol. Syst. Biol. 5: 275). Plasmids were maintained in the host E. coli DH5alpha (F⁻, phi80dlacZdeltaM15, delta(lacZYA-argF)U169, deoR, recA1, endA1, hsdR17(rk⁻, mk⁺), phoA, supE44, lambda⁻, thi-1, gyrA96, relAl) bought from Invitrogen.

C. Bacillus subtilis

### Media and Cultivation

Two media are used to cultivate B. subtilis: i.e., a complex medium like a rich Luria Broth (LB) and a minimal medium for shake flask cultures. The LB medium consisted of 1% tryptone peptone (Difco), 0.5% yeast extract (Difco) and 0.5% sodium chloride (VWR). Luria Broth agar (LBA) plates consisted of the LB media, with 12 g/L agar (Difco) added. The minimal medium contained 2.00 g/L (NH₄)₂SO₄, 7.5 g/L KH₂PO₄, 17.5 g/L K₂HPO₄, 1.25 g/L Na-citrate, 0.25 g/L MgSO₄·7H₂O, 0.05 g/L tryptophan, from 10 up to 30 g/L glucose (or another carbon source including but not limited to fructose, maltose, sucrose, glycerol and maltotriose), 10 mL/L trace element mix and 10 mL/L Fe-citrate solution. The medium was set to a pH of 7 with 1 M KOH. As precursor(s) and/or acceptor(s) for saccharide synthesis, compounds like e.g., galactose, glucose, fructose, fucose, lactose, LacNAc, LNB, a co-factor and sialic acid could be added to the medium. The trace element mix consisted of 0.735 g/L CaCl₂·2H₂O, 0.1 g/L MnCl₂·2H₂O, 0.033 g/L CuCl₂·2H₂O, 0.06 g/L CoCl₂·6H₂O, 0.17 g/L ZnCl₂, 0.0311 g/L H₃BO₄, 0.4 g/L Na₂EDTA·2H₂O and 0.06 g/L Na₂MoO₄. The Fe-citrate solution contained 0.135 g/L FeCl₃·6H₂O, 1 g/L Na-citrate (Hoch 1973 PMC1212887). Complex medium, e.g., LB, was sterilized by autoclaving (121° C., 21 min) and minimal medium by filtration (0.22 μm Sartorius). When necessary, the medium was made selective by adding an antibiotic. B. subtilis strains were initially grown on LB agar to obtain single colonies. These plates were grown over night at 37° C. Starting from a single colony, a preculture was grown over night in 5 mL at 37° C., shaking at 200 rpm. Subsequent 125 mL shake flask experiments were inoculated with 2% of this preculture, in 25 mL media. These shake flasks were incubated at 37° C. with an orbital shaking of 200 rpm for 72 h, or shorter of longer. At the end of the cultivation experiment samples were taken to measure the supernatant concentration (extracellular sugar concentrations, after 5 min. spinning down the cells), or by boiling the culture broth for 15 min at 90° C. or for 60 min at 60° C. before spinning down the cells (=whole broth concentration, i.e., intra- and extracellular sugar concentrations).

Also, a dilution of the cultures was made to measure the optical density at 600 nm. The cell performance index or CPI was determined by dividing the oligosaccharide concentrations by the biomass, in relative percentages compared to a reference strain. The biomass is empirically determined to be approximately ⅓rd of the optical density measured at 600 nm.

### Strains, Plasmids and Mutations

B. subtilis 168 is used as available at the Bacillus Genetic Stock Center (Ohio, USA). Plasmids for gene deletion via Cre/lox are constructed as described by Yan et al. (Appl & Environm microbial, September 2008, p5556-5562). Gene disruption is done via homologous recombination with linear DNA and transformation via the electroporation as described by Xue et al. (J. microb. Meth. 34 (1999) 183-191). The method of gene knockouts is described by Liu et al. (Metab. Engine. 24 (2014) 61-69). Integrative vectors as described by Popp et al. (Sci. Rep., 2017, 7, 15158) are used as expression vector and could be further used for genomic integrations if necessary. A suitable promoter for expression can be derived from the part repository (iGem): sequence id: BBa_K143012, BBa_K823000, BBa_K823002 or BBa_K823003. Cloning can be performed using Gibson Assembly, Golden Gate assembly, Cliva assembly, LCR or restriction ligation.

In an example for the production of lactose-based oligosaccharides, Bacillus subtilis mutant strains are created to contain a gene coding for a lactose importer (such as e.g., the E. coli lacY with UniProt ID P02920).

In an example for the production of LN3, the B. subtilis strain is modified with a genomic knock-in of constitutive transcriptional units comprising a lactose importer (such as e.g., the E. coli lacY with UniProt ID P02920) and a galactoside beta-1,3-N-acetylglucosaminyltransferase like e.g., LgtA from N. meningitidis (UniProt ID Q9JXQ6). For LNT production, the LN3 producing strain is further modified with a constitutive transcriptional unit for an N-acetylglucosamine beta-1,3-galactosyltransferase like e.g., WbgO from E. coli 055:H7 (UniProt ID D3QY14). For LNnT production, the LN3 producing strain is further modified with a constitutive transcriptional unit for an N-acetylglucosamine beta-1,4-galactosyltransferase like e.g., LgtB from N. meningitidis (UniProt ID Q51116, sequence version 02, 1 Dec. 2000). The N-acetylglucosamine beta-1,3-galactosyltransferase and the N-acetylglucosamine beta-1,4-galactosyltransferase can be delivered to the strain either via genomic knock-in or from an expression plasmid.

To produce a fucosylated saccharide, the B. subtilis strains are modified with a constitutive transcriptional unit for a fucosyltransferase.

In an example for sialic acid production, a mutant B. subtilis strain is created by overexpressing a fructose-6-P-aminotransferase like the native fructose-6-P-aminotransferase glmS (UniProt ID P0CI73) to enhance the intracellular glucosamine-6-phosphate pool. Further on, the enzymatic activities of the genes nagA, nagB and gamA are disrupted by genetic knockouts and a glucosamine-6-P-aminotransferase like e.g., GNA1 from S. cerevisiae (UniProt ID P43577), an N-acetylglucosamine-2-epimerase like e.g., from B. ovatus (UniProt ID A7LVG6) and an N-acetylneuraminate synthase like e.g., from N. meningitidis (UniProt ID E0NCD4) are overexpressed on the genome. In another example to produce sialic acid, a mutant B. subtilis strain is created by genetic knockouts of the genes nagA, nagB and gamA and by overexpressing a fructose-6-P-aminotransferase like the native fructose-6-P-aminotransferase glmS (UniProt ID P0CI73), a hydrolyzing UDP-N-acetylglucosamine 2-epimerase like e.g., NeuC from C. jejuni (UniProt ID Q93MP8) or any one of SEQ ID NO:01, 02, 03, 04, 05, 06, 07 or 08, and an N-acetylneuraminate synthase like e.g., NeuB from N. meningitidis (UniProt ID E0NCD4). To allow sialylated saccharide production, the sialic acid producing strain is further modified with a constitutive transcriptional unit comprising an N-acylneuraminate cytidylyltransferase like e.g., the NeuA enzyme from P. multocida (UniProt ID A0A849CI62), and a sialyltransferase like e.g., an alpha-2,3-sialyltransferase like e.g., the alpha-2,3-sialyltransferase PmultST3 from P. multocida (UniProt ID Q9CLP3) or a PmultST3-like polypeptide (SEQ ID NO: 10) consisting of amino acid residues 1 to 268 of UniProt ID Q9CLP3 having beta-galactoside alpha-2,3-sialyltransferase activity or an alpha-2,6-sialyltransferase like e.g., the alpha-2,6-sialyltransferase (PdST6) from P. damselae (UniProt ID 066375) or a PdST6-like polypeptide (SEQ ID NO: 11) consisting of amino acid residues 108 to 497 of UniProt ID 066375 having beta-galactoside alpha-2,6-sialyltransferase activity.

For growth on sucrose, the mutant strains can additionally be modified with genomic knock-ins of constitutive transcriptional units comprising the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6). Optionally, a constitutive transcriptional unit for a siderophore transporter like e.g., entS from E. coli (UniProt ID P24077, sequence version 02 (1 Nov. 1997)) is added as well.

D. Corynebacterium glutamicum

### Media and Cultivation

Two different media are used, namely complex medium like e.g., a rich tryptone-yeast extract (TY) medium, and a minimal medium for shake flask (MMsf). The minimal medium uses a 1000× stock trace element mix. Trace element mix consisted of 10 g/L CaCl₂, 10 g/L FeSO₄·7H₂O, 10 g/L MnSO₄·H₂O, 1 g/L ZnSO₄·7H₂O, 0.2 g/L CuSO₄, 0.02 g/L NiCl₂·6H₂O, 0.2 g/L biotin (pH 7) and 0.03 g/L protocatechuic acid. The minimal medium for the shake flasks (MMsf) experiments contained 20 g/L (NH₄)₂SO₄, 5 g/L urea, 1 g/L KH₂PO₄, 1 g/L K₂HPO₄, 0.25 g/L MgSO₄·7H₂O, 42 g/L MOPS, from 10 up to 30 g/L glucose or another carbon source including but not limited to fructose, maltose, sucrose, glycerol and maltotriose when specified in the examples and 1 ml/L trace element mix. As precursor(s) and/or acceptor(s) for saccharide synthesis, compounds like e.g., galactose, glucose, fructose, fucose, lactose, LacNAc, LNB, a co-factor and sialic acid could be added to the medium. The TY medium consisted of 1.6% tryptone (Difco, Erembodegem, Belgium), 1% yeast extract (Difco) and 0.5% sodium chloride (VWR. Leuven, Belgium). TY agar (TYA) plates consisted of the TY media, with 12 g/L agar (Difco, Erembodegem, Belgium) added. Complex medium, e.g., TY, was sterilized by autoclaving (121° C., 21 min) and minimal medium by filtration (0.22 μm Sartorius). When necessary, the medium was made selective by adding an antibiotic.

A preculture was started from a cryovial or a single colony from a TY plate, in 6 mL TY and was incubated overnight at 37° C. on an orbital shaker at 200 rpm. Subsequent 125 mL shake flask experiments were inoculated with 2% of this preculture, in 25 mL MMsf medium. These shake flasks were incubated at 37° C. with an orbital shaking of 200 rpm for 72 h, or shorter of longer. At the end of the cultivation experiment samples were taken to measure the supernatant concentration (extracellular sugar concentrations, after 5 min. spinning down the cells), or by boiling the culture broth for 15 min at 90° C. or for 60 min at 60° C. before spinning down the cells (=whole broth concentration, i.e., intra- and extracellular sugar concentrations). Also, a dilution of the cultures was made to measure the optical density at 600 nm. The cell performance index or CPI was determined by dividing the oligosaccharide concentrations, e.g., 3′SL concentrations, measured in the whole broth by the biomass, in relative percentages compared to the reference strain. The biomass is empirically determined to be approximately ⅓rd of the optical density measured at 600 nm.

### Strains and Mutations

Corynebacterium glutamicum was used as available at the American Type Culture Collection (ATCC 13032). Integrative plasmid vectors were made using the Cre/loxP technique as described by Suzuki et al. (Appl. Microbiol. Biotechnol., 2005 April, 67(2):225-33) and temperature-sensitive shuttle vectors as described by Okibe et al. (Journal of Microbiological Methods 85, 2011, 155-163) are constructed for gene deletions, mutations and insertions. Suitable promoters for (heterologous) gene expression can be derived from Yim et al. (Biotechnol. Bioeng., 2013 November, 110(11):2959-69). Cloning can be performed using Gibson Assembly, Golden Gate assembly, Cliva assembly, LCR or restriction ligation.

In an example for the production of lactose-based oligosaccharides, C. glutamicum mutant strains are created to contain a gene coding for a lactose importer (such as e.g., the E. coli lacY with UniProt ID P02920).

In an example for the production of LN3, the C. glutamicum strain is modified with a genomic knock-in of constitutive expression units comprising a lactose importer (such as e.g., the E. coli lacY with UniProt ID P02920) and a galactoside beta-1,3-N-acetylglucosaminyltransferase like e.g., LgtA from N. meningitidis (UniProt ID Q9JXQ6). For LNT production, the LN3 producing strain is further modified with a constitutive transcriptional unit for an N-acetylglucosamine beta-1,3-galactosyltransferase like e.g., WbgO from E. coli O55:H7 (UniProt ID D3QY14). For LNnT production, the LN3 producing strain is further modified with a constitutive transcriptional unit for an N-acetylglucosamine beta-1,4-galactosyltransferase like e.g., LgtB from N. meningitidis (UniProt ID Q51116, sequence version 02, 1 Dec. 2000). The N-acetylglucosamine beta-1,3-galactosyltransferase and the N-acetylglucosamine beta-1,4-galactosyltransferase can be delivered to the strain either via genomic knock-in or from an expression plasmid.

To further produce a fucosylated saccharide, the mutant C. glutamicum strains are further modified with a constitutive transcriptional unit for a fucosyltransferase.

In an example for sialic acid production, a mutant C. glutamicum strain is created by overexpressing a fructose-6-P-aminotransferase like the native fructose-6-P-aminotransferase glmS (UniProt ID Q8NND3, sequence version 03, 23 Jan. 2007) to enhance the intracellular glucosamine-6-phosphate pool. Further on, the enzymatic activities of the genes nagA, nagB and gamA are disrupted by genetic knockouts and a glucosamine-6-P-aminotransferase like e.g., GNA1 from S. cerevisiae (UniProt ID P43577), an N-acetylglucosamine-2-epimerase like e.g., from B. ovatus (UniProt ID A7LVG6) and an N-acetylneuraminate synthase like e.g., from N. meningitidis (UniProt ID E0NCD4) are overexpressed on the genome. In another example to produce sialic acid, a mutant C. glutamicum strain is created by genetic knockouts of the genes nagA, nagB and gamA and by overexpressing a fructose-6-P-aminotransferase like the native fructose-6-P-aminotransferase glmS (UniProt ID Q8NND3, sequence version 03, 23 Jan. 2007), a hydrolyzing UDP-N-acetylglucosamine 2-epimerase like e.g., NeuC from C. jejuni (UniProt ID Q93MP8) or any one of SEQ ID NO:01, 02, 03, 04, 05, 06, 07 or 08, and an N-acetylneuraminate synthase like e.g., NeuB from N. meningitidis (UniProt ID E0NCD4). To allow sialylated saccharide production, the sialic acid producing strain is further modified with a constitutive transcriptional unit comprising an N-acylneuraminate cytidylyltransferase like e.g., the NeuA enzyme from P. multocida (UniProt ID A0A849CI62), and a sialyltransferase like e.g., an alpha-2,3-sialyltransferase like e.g., the alpha-2,3-sialyltransferase PmultST3 from P. multocida (UniProt ID Q9CLP3) or a PmultST3-like polypeptide (SEQ ID NO: 10) consisting of amino acid residues 1 to 268 of UniProt ID Q9CLP3 having beta-galactoside alpha-2,3-sialyltransferase activity or an alpha-2,6-sialyltransferase like e.g., the alpha-2,6-sialyltransferase (PdST6) from P. damselae (UniProt ID 066375) or a PdST6-like polypeptide (SEQ ID NO: 11) consisting of amino acid residues 108 to 497 of UniProt ID 066375 having beta-galactoside alpha-2,6-sialyltransferase activity. If the engineered strains producing sialic acid and CMP-sialic acid were intended to make sialylated lactose structures, the strains were additionally modified with a genomic knock-in of a constitutive transcriptional unit for a lactose permease like e.g., the E. coli LacY (UniProt ID P02920).

For growth on sucrose, the mutant strains can additionally be modified with genomic knock-ins of constitutive transcriptional units comprising the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6).

E. Chlamydomonas reinhardtii

### Media and Cultivation

Chlamydomonas reinhardtii cells were cultured in Tris-acetate-phosphate (TAP) medium (pH 7). The TAP medium uses a 1000× stock Hutner's trace element mix. Hutner's trace element mix consisted of 50 g/L Na2EDTA·H2O (Titriplex III), 22 g/L ZnSO₄·7H2O, 11.4 g/L H3BO3, 5 g/L MnCl2·4H2O, 5 g/L FeSO4·7H2O, 1.6 g/L CoCl2·6H2O, 1.6 g/L CuSO4·5H2O and 1.1 g/L (NH4)6MoO3. The TAP medium contained 2.42 g/L Tris (tris(hydroxymethyl)aminomethane), 25 mg/L salt stock solution, 0.108 g/L K2HPO4, 0.054 g/L KH2PO4 and 1.0 mL/L glacial acetic acid. The salt stock solution consisted of 15 g/L NH4CL, 4 g/L MgSO4·7H2O and 2 g/L CaCl2)·2H2O. As precursor(s) and/or acceptor(s) for saccharide synthesis, compounds like e.g., galactose, glucose, fructose, fucose, lactose, LacNAc, LNB, a co-factor and sialic acid could be added. Medium was sterilized by autoclaving (121° C., 21 min). For stock cultures on agar slants TAP medium was used containing 1% agar (of purified high strength, 1000 g/cm2).

Cells of C. reinhardtii were cultured in selective TAP-agar plates at 23+/−0.5° C. under 14/10 h light/dark cycles with a light intensity of 8000 Lx. Cells were analyzed after 5 to 7 days of cultivation. For high-density cultures, cells could be cultivated in closed systems like e.g., vertical or horizontal tube photobioreactors, stirred tank photobioreactors or flat panel photobioreactors as described by Chen et al. (Bioresour. Technol. 2011, 102: 71-81) and Johnson et al. (Biotechnol. Prog. 2018, 34: 811-827).

### Strains, Plasmids and Mutations

C. reinhardtii wild-type strains 21 gr (CC-1690, wild-type, mt+), 6145C (CC-1691, wild-type, mt−), CC-125 (137c, wild-type, mt+), CC-124 (137c, wild-type, mt−) as available from the Chlamydomonas Resource Center (https://www.chlamycollection.org) (University of Minnesota, U.S.A.) were used. Expression plasmids originated from pSI103, as available from the Chlamydomonas Resource Center. Cloning can be performed using Gibson Assembly, Golden Gate assembly, Cliva assembly, LCR or restriction ligation. Suitable promoters for (heterologous) gene expression can be derived from e.g., Scranton et al. (Algal Res. 2016, 15: 135-142). Targeted gene modification (like gene knock-out or gene replacement) can be carried using the Crispr-Cas technology as described e.g., by Jiang et al. (Eukaryotic Cell 2014, 13(11): 1465-1469). Transformation via electroporation was performed as described by Wang et al. (Biosci. Rep. 2019, 39: BSR2018210) and as described like e.g., in WO22034067 or in WO22034069.

In an example for sialic acid and CMP-sialic acid production, the mutant strain was derived from C. reinhardtii and modified as described e.g., in WO22034067. Optionally, the mutant strain is modified with a transcriptional unit for an additional hydrolyzing UDP-N-acetylglucosamine-2-epimerase like e.g., any one of SEQ ID NO:01, 02, 03, 04, 05, 06, 07 or 08. In an example for production of sialylated oligosaccharides, C. reinhardtii cells are modified with a CMP-sialic acid transporter like e.g., CST from Mus musculus (UniProt ID Q61420), and a Golgi-localized sialyltransferase selected from species like e.g., Homo sapiens, Mus musculus, Rattus norvegicus. In an example for GDP-fucose synthesis, the mutant strain was derived from C. reinhardtii and modified as described e.g., in WO22034067. In an example for fucosylation, C. reinhardtii cells can be modified with an expression plasmid comprising a constitutive transcriptional unit for an alpha-1,2-fucosyltransferase and/or an alpha-1,3-fucosyltransferase. In an example for UDP-galactose synthesis, the mutant strain was derived from C. reinhardtii and modified as described e.g., in WO22034067. In an example for LN3 production, the mutant strain was derived from C. reinhardtii and modified as described e.g., in WO22034067 to comprise a transcriptional unit for a galactoside beta-1,3-N-acetylglucosaminyltransferase like e.g., LgtA from N. meningitidis (UniProt ID Q9JXQ6). In an example for LNT or LNnT production, the LN3 producing strain is further modified with a constitutive transcriptional unit comprising an N-acetylglucosamine beta-1,3-galactosyltransferase like e.g., WbgO (Uniprot ID D3QY14) from E. coli 055:H7 or an N-acetylglucosamine beta-1,4-galactosyltransferase like e.g., LgtB (Uniprot ID Q51116, sequence version 02, 1 Dec. 2000) from N. meningitidis, respectively. In an example to produce one or more fucosylated non-charged oligosaccharide(s), a C. reinhardtii strain is modified for production of GDP-fucose, UDP-galactose, LN3, LNT and/or LNnT as described herein and for expression of one or more compatible fucosyltransferase(s). In an example to produce one or more sialylated oligosaccharide(s) like e.g., LSTa and LSTb, a C. reinhardtii strain is modified for production of CMP-sialic acid, UDP-galactose, LN3 and LNT as described herein and for expression of one or more compatible sialyltransferase(s) like e.g., an alpha-2,3-sialyltransferase like e.g., the alpha-2,3-sialyltransferase PmultST3 from P. multocida (UniProt ID Q9CLP3) or a PmultST3-like polypeptide (SEQ ID NO: 10) consisting of amino acid residues 1 to 268 of UniProt ID Q9CLP3 having beta-galactoside alpha-2,3-sialyltransferase activity or an alpha-2,6-sialyltransferase like e.g., the alpha-2,6-sialyltransferase (PdST6) from P. damselae (UniProt ID 066375) or a PdST6-like polypeptide (SEQ ID NO: 11) consisting of amino acid residues 108 to 497 of UniProt ID 066375 having beta-galactoside alpha-2,6-sialyltransferase activity. In an example to produce one or more sialylated oligosaccharide(s) like e.g., LSTc and LSTd, a C. reinhardtii strain is modified for production of CMP-sialic acid, UDP-galactose, LN3 and LNnT as described herein and for expression of one or more compatible sialyltransferase(s).

### F. Animal Cells

Isolation of Mesenchymal Stem Cells from Adipose Tissue of Different Animals

Fresh adipose tissue is obtained from slaughterhouses (e.g., cattle, pigs, sheep, chicken, ducks, catfish, snake, frogs) or liposuction (e.g., in case of humans, after informed consent) and kept in phosphate buffer saline supplemented with antibiotics. Enzymatic digestion of the adipose tissue is performed followed by centrifugation to isolate mesenchymal stem cells. The isolated mesenchymal stem cells are transferred to cell culture flasks and grown under standard growth conditions, e.g., 37° C., 5% CO2. The initial culture medium includes DMEM-F12, RPMI, and Alpha-MEM medium (supplemented with 15% fetal bovine serum), and 1% antibiotics. The culture medium is subsequently replaced with 10% FBS (fetal bovine serum)-supplemented media after the first passage. For example, Ahmad and Shakoori (2013, Stem Cell Regen. Med. 9(2): 29-36), which is incorporated herein by reference in its entirety for all purposes, describes certain variation(s) of the method(s) described herein in this example.

Isolation of Mesenchymal Stem Cells from Milk

This example illustrates isolation of mesenchymal stem cells from milk collected under aseptic conditions from human or any other mammal(s) such as described herein. An equal volume of phosphate buffer saline is added to diluted milk, followed by centrifugation for 20 min. The cell pellet is washed thrice with phosphate buffer saline and cells are seeded in cell culture flasks in DMEM-F12, RPMI, and Alpha-MEM medium supplemented with 10% fetal bovine serum and 1% antibiotics under standard culture conditions. For example, Hassiotou et al. (2012, Stem Cells. 30(10): 2164-2174), which is incorporated herein by reference in its entirety for all purposes, describes certain variation(s) of the method(s) described herein in this example.

### Differentiation of Stem Cells Using 2D and 3D Culture Systems

The mesenchymal cells isolated from adipose tissue of different animals or from milk as described above can be differentiated into mammary-like epithelial and luminal cells in 2D and 3D culture systems. See, for example, Huynh et al. 1991. Exp Cell Res. 197(2): 191-199; Gibson et al. 1991, In Vitro Cell Dev Biol Anim. 27(7): 585-594; Blatchford et al. 1999; Animal Cell Technology′: Basic & Applied Aspects, Springer, Dordrecht. 141-145; Williams et al. 2009, Breast Cancer Res 11(3): 26-43; and Arevalo et al. 2015, Am J Physiol Cell Physiol. 310(5): C348-C356; each of which is incorporated herein by reference in their entireties for all purposes.

For 2D culture, the isolated cells were initially seeded in culture plates in growth media supplemented with 10 ng/mL epithelial growth factor and 5 pg/mL insulin. At confluence, cells were fed with growth medium supplemented with 2% fetal bovine serum, 1% penicillin-streptomycin (100 U/mL penicillin, 100 ug/mL streptomycin), and 5 pg/mL insulin for 48 h. To induce differentiation, the cells were fed with complete growth medium containing 5 pg/mL insulin, 1 pg/mL hydrocortisone, 0.65 ng/mL triiodothyronine, 100 nM dexamethasone, and 1 pg/mL prolactin. After 24 h, serum is removed from the complete induction medium.

For 3D culture, the isolated cells were trypsinized and cultured in Matrigel, hyaluronic acid, or ultra-low attachment surface culture plates for six days and induced to differentiate and lactate by adding growth media supplemented with 10 ng/mL epithelial growth factor and 5 pg/mL insulin. At confluence, cells were fed with growth medium supplemented with 2% fetal bovine serum, 1% penicillin-streptomycin (100 U/mL penicillin, 100 ug/mL streptomycin), and 5 pg/mL insulin for 48 h. To induce differentiation, the cells were fed with complete growth medium containing 5 pg/mL insulin, 1 pg/mL hydrocortisone, 0.65 ng/mL triiodothyronine, 100 nM dexamethasone, and 1 pg/mL prolactin. After 24 h, serum is removed from the complete induction medium.

### Method of Making Mammary-Like Cells

In a next step, the cells are brought to induced pluripotency by reprogramming with viral vectors encoding for Oct4, Sox2, Klf4, and c-Myc. The resultant reprogrammed cells are then cultured in Mammocult media (available from Stem Cell Technologies), or mammary cell enrichment media (DMEM, 3% FBS, estrogen, progesterone, heparin, hydrocortisone, insulin, EGF) to make them mammary-like, from which expression of select milk components can be induced. Alternatively, epigenetic remodeling is performed using remodeling systems such as CRISPR/Cas9, to activate select genes of interest, such as casein, a-lactalbumin to be constitutively on, to allow for the expression of their respective proteins, and/or to down-regulate and/or knock-out select endogenous genes as described e.g., in WO21067641, which is incorporated herein by reference in its entirety for all purposes. In an example for production of one or more oligosaccharide(s), isolated mesenchymal cells re-programmed into mammary-like cells are modified via CRISPR-CAS as described e.g., in WO22034067, WO22034070 and WO22034075.

### Cultivation

Completed growth media includes high glucose DMEM/F12, 10% FBS, 1% NEAA, 1% pen/strep, 1% ITS-X, 1% F-Glu, 10 ng/mL EGF, and 5 μg/mL hydrocortisone. Completed lactation media includes high glucose DMEM/F12, 1% NEAA, 1% pen/strep, 1% ITS-X, 1% F-Glu, 10 ng/mL EGF, 5 μg/mL hydrocortisone, and 1 μg/mL prolactin (5 ug/mL in Hyunh 1991). Cells are seeded at a density of 20,000 cells/cm2 onto collagen coated flasks in completed growth media and left to adhere and expand for 48 hours in completed growth media, after which the media is switched out for completed lactation media. Upon exposure to the lactation media, the cells start to differentiate and stop growing. Within about a week, the cells start secreting lactation product(s) such as milk lipids, lactose, casein and whey into the media. A desired concentration of the lactation media can be achieved by concentration or dilution by ultrafiltration. A desired salt balance of the lactation media can be achieved by dialysis, for example, to remove unwanted metabolic products from the media. Hormones and other growth factors used can be selectively extracted by resin purification, for example, the use of nickel resins to remove His-tagged growth factors, to further reduce the levels of contaminants in the lactated product.

### G. Optical Density

Cell density of the cultures was frequently monitored by measuring optical density at 600 nm (Implen Nanophotometer NP80, Westburg, Belgium or with a Spark 10M microplate reader, Tecan, Switzerland). The maximum growth speed (mumax) was calculated based on the observed optical densities at 600 nm using the R package grofit.

### H. Growth Rate Speed Measurement

The maximal growth rate (μMax) was calculated based on the observed optical densities at 600 nm using the R package grofit.

### I. Heterologous and Homologous Expression

Genes that needed to be expressed, be it from a plasmid or from the genome were synthetically synthetized with one of the following companies: IDT or Twist Bioscience. Proteins described in present disclosure are summarized in Table 1. Unless stated otherwise, the UniProt IDs of the proteins described correspond to their sequence version 01 as present in the UniProt Database version release 2021_03 of 9 Jun. 2021. Expression could be further facilitated by optimizing the codon usage to the codon usage of the expression host. Genes were optimized using the tools of the supplier.

TABLE 1
Overview of proteins with corresponding SEQ ID NOs or UniProt IDs (sequence
version 01, UniProt Database 2021_03 of Jun. 9, 2021) as described in present disclosure.

| | | | | Country of origin of |
|---|---|---|---|---|
| | | | | digital sequence |
| SEQ ID NO | Name | Organism | Origin | information |
| 01 | nnaA | Algoriphagus sanaruensis | Synthetic | Japan |
| 02 | nnaA | Pseudoalteromonas | Synthetic | USA |
| | | luteoviolacea | | |
| 03 | nnaA | Campylobacter upsaliensis | Synthetic | Unknown |
| | | JV21 | | |
| 04 | nnaA | Helicobacter felis | Synthetic | Australia |
| 05 | nnaA | Neisseria weaveri | Synthetic | Sweden |
| 06 | nnaA | Halomonas meridiana | Synthetic | USA |
| 07 | nnaA | Parcubacteria group | Synthetic | USA |
| | | bacterium | | |
| | | GW2011_GWA2_38_13 | | |
| 08 | nnaA | Flavobacterium sp. (strain | Synthetic | USA |
| | | CF136) | | |
| 09 | glmS*54 | Escherichia coli K-12 | Synthetic | USA |
| | | MG1655 | | |
| 10 | PmultST3-like | Pasteurella multocida | Synthetic | USA |
| | polypeptide | | | |
| 11 | PdST6-like | Photobacterium damselae | Synthetic | Japan |
| | polypeptide | | | |
| 12 | Conserved motif ^{▪} | N.A. | Synthetic | N.A. |
| A0A1B1U4V1 | 2FT | Helicobacter sp. MIT 01-6242 | Synthetic | USA |
| Z4WWI2 | 3FT | Porphyromonas catoniae | Synthetic | USA |
| P27550 | acs | Escherichia coli K-12 | Synthetic | USA |
| | | MG1655 | | |
| A7LVG6 | AGE | Bacteroides ovatus | Synthetic | USA |
| A0ZZH6 | BaSP | Bifidobacterium adolescentis | Synthetic | Germany |
| E0IXR1 | cscB | Escherichia coli W | Synthetic | USA |
| Q61420 | CST | Mus musculus | Synthetic | USA |
| P24077* | entS | Escherichia coli K-12 | Synthetic | USA |
| | | MG1655 | | |
| P32055* | fcl | Escherichia coli K-12 | Synthetic | USA |
| | | MG1655 | | |
| SUV40286.1 | fkp | Bacteroides fragilis | Synthetic | United Kingdom |
| Q03417 | Frk | Zymomonas mobilis | Synthetic | United Kingdom |
| P11551** | fucP | Escherichia coli K-12 | Synthetic | USA |
| | | MG1655 | | |
| B9YZ84 | furA | Pseudogulbenkiania | Synthetic | USA |
| | | ferrooxidans | | |
| P09147 | galE | Escherichia coli K-12 | Synthetic | USA |
| | | MG1655 | | |
| Q9RHG8 | galT | Helicobacter pylori UA1234 | Synthetic | Canada |
| P15877*** | gcd | Escherichia coli K-12 | Synthetic | USA |
| | | MG1655 | | |
| P31120**** | glmM | Escherichia coli K-12 | Synthetic | USA |
| | | MG1655 | | |
| P0CI73 | glmS | Bacillus subtilis | Synthetic | USA |
| Q8NND3***** | glmS | Corynebacterium glutamicum | Synthetic | USA |
| P17169° | glmS | Escherichia coli K-12 | Synthetic | USA |
| | | MG1655 | | |
| P14192 | glmU | Bacillus subtilis | Synthetic | USA |
| P0ACC7 | glmU | Escherichia coli K-12 | Synthetic | USA |
| | | MG1655 | | |
| P0AC88 | gmd | Escherichia coli K-12 | Synthetic | USA |
| | | MG1655 | | |
| P43577 | GNA1 | Saccharomyces cerevisiae | Synthetic | USA |
| Q91WG8 | GNE | Mus musculus | Synthetic | USA |
| 030511 | HpFucT | Helicobacter pylori UA1234 | Synthetic | Canada |
| Q9X435 | HpFutC | Helicobacter pylori UA1234 | Synthetic | Canada |
| P07921 | LAC12 | Kluyveromyces lactis | Synthetic | USA |
| P02920 | LacY | Escherichia coli K-12 | Synthetic | USA |
| | | MG1655 | | |
| Q9JXQ6 | LgtA | Neisseria meningitidis MC58 | Synthetic | United Kingdom |
| Q51116°° | LgtB | Neisseria meningitidis MC58 | Synthetic | United Kingdom |
| P00946 | manA | Escherichia coli K-12 | Synthetic | USA |
| | | MG1655 | | |
| P24175 | manB | Escherichia coli K-12 | Synthetic | USA |
| | | MG1655 | | |
| P24174°°° | manC | Escherichia coli K-12 | Synthetic | USA |
| | | MG1655 | | |
| Q8A712 | NANP | Bacteroides thetaiotaomicron | Synthetic | Unknown |
| K9NPH9 | NANS | Pseudomonas sp. UW4 | Synthetic | Unknown |
| Q93MP7 | NeuA | Campylobacter jejuni | Synthetic | USA |
| A0A849CI62 | NeuA | Pasteurella multocida | Synthetic | USA |
| E0NCD4 | NeuB | Neisseria meningitidis | Synthetic | United Kingdom |
| Q93MP8 | NeuC | Campylobacter jejuni | Synthetic | USA |
| O66375 | PdST6 | Photobacterium damselae | Synthetic | Japan |
| Q9CLP3 | PmultST3 | Pasteurella multocida | Synthetic | USA |
| P0A840 | SurE | Escherichia coli K-12 | Synthetic | USA |
| | | MG1655 | | |
| Q5UHA8 | wbdO | Salmonella enterica | Synthetic | United Kingdom |
| D3QY14 | wbgO | Escherichia coli 055:H7 | Synthetic | Germany |

*Sequence version 02 (Nov. 1, 1997) as present in the UniProt Database 2021_03 of Jun. 9, 2021
**Sequence version 03 (Jan. 23, 2007) as present in the UniProt Database 2021 03 of Jun. 9, 2021
***Sequence version 03 (Dec. 2, 2020) as present in the UniProt Database 2021_03 of Jun. 9, 2021
****Sequence version 03 (Jan. 23, 2007) as present in the UniProt Database 2021_03 of Jun. 9, 2021
*****Sequence version 02 (Jan. 23, 2007) as present in the UniProt Database 2021_03 of Jun. 9, 2021
°Sequence version 04 (Jan. 23, 2007) as present in the UniProt Database 2021_03 of Jun. 9, 2021
°°Sequence version 02 (Dec. 1, 2000) as present in the UniProt Database 2021_03 of Jun. 9, 2021
°°°Sequence version 03 (Jul. 19, 2003) as present in the UniProt Database 2021_03 of Jun. 9, 2021
^{▪} Conserved motif = [ACILM][GST]N[ST][ST]XXXX[DE][ACGILMST][ACDEGIMPQSTV] = SEQ ID NO: 12

### J. Analytical Analysis

Standards such as but not limited to sucrose, lactose, 3′SL, 6′SL, lacto-N-triose II (LN3), lacto-N-tetraose (LNT), lacto-N-neo-tetraose (LNnT), LNFP-I, LNFP-II, LNFP-III, LNFP-V, LNFP-VI, LSTa, LSTc and LSTd were purchased from Carbosynth (UK), Elicityl (France) and IsoSep (Sweden). Other compounds were analyzed with in-house made standards.

Neutral oligosaccharides were analyzed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 μm) column with an Acquity UPLC BEH Amide VanGuard column, 130 Å, 2.1×5 mm. The column temperature was 50° C. The mobile phase consisted of a ¼ water and ¾ acetonitrile solution to which 0.2% triethylamine was added. The method was isocratic with a flow of 0.130 mL/min. The ELSD detector had a drift tube temperature of 50° C. and the N2 gas pressure was 50 psi, the gain 200 and the data rate 10 pps. The temperature of the RI detector was set at 35° C.

Sialylated oligosaccharides were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C.

Both neutral and sialylated sugars were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionisation (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2% of eluent B was restored in 1 min and maintained for 12 min.

Both neutral and sialylated sugars at low concentrations (below 50 mg/L) were analyzed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 min while maintaining a constant ratio of 25% of eluent B using the following gradient: an initial isocratic step maintained for 10 min of 75% of eluent A, an initial increase from 0 to 4% of eluent C over 8 min, a second isocratic step maintained for 6 min of 71% of eluent A and 4% of eluent C, a second increase from 4 to 12% of eluent C over 2.6 min, a third isocratic step maintained for 3.4 min of 63% of eluent A and 12% of eluent C and a third increase from 12 to 48% of eluent C over 5 min. As a washing step 48% of eluent C was used for 3 min. For column equilibration, the initial condition of 75% of eluent A and 0% of eluent C was restored in 1 min and maintained for 11 min. The applied flow was 0.5 mL/min.

Lactobionic acid was analyzed on a Dionex HPAEC system with pulsed amperometric detection (PAD). A volume of 5 μL of sample was injected on a Dionex CarboPac PA01 column 2×250 mm with a Dionex CarboPac PA01 guard column 4×50 mm. The column temperature was set to 30° C. A gradient was used wherein eluent A was ultrapure water, eluent B was 200 mM Sodium hydroxide and eluent C was 500 mM Sodium acetate. Total gradient time was 41 min and started with 50% B and 5% C in the first 7 minutes. During the next 18 minutes, concentration slowly changed to 50% B and 40% C at 25 minutes, followed by a rinse step with 100% C for 7 minutes. For column equilibration, the initial condition with 50% B and 5% C was restored in 9 minutes. The applied flow was 0.25 mL/min.

### Example 2. Evaluation of Production of Sialic Acid and 6′SL with a Modified E. coli Host

An E. coli K-12 MG1655 strain was modified for production of sialic acid comprising genomic knock-ins of constitutive transcriptional units containing nucleotide sequences encoding the mutant glmS*54 from E. coli (SEQ ID NO:09) (differing from the wild-type E. coli glmS, having UniProt ID P17169, by an A39T, an R250C and an G472S mutation as described by Deng et al. (Biochimie 88, 419-29 (2006)), a hydrolyzing UDP-N-acetylglucosamine-2-epimerase selected from the list comprising SEQ ID NO:01, 02, 03, 04, 05, 06, 07, 08 and the reference polypeptide NeuC from Campylobacter jejuni (UniProt ID Q93MP8), and the N-acetylneuraminate synthase NeuB from N. meningitidis (UniProt ID E0NCD4). To allow production of 6′SL, the strains were further modified with genomic knock-ins of two constitutive transcriptional units containing nucleotide sequences encoding the N-acylneuraminate cytidylyltransferase neuA from P. multocida (UniProt ID A0A849CI62) and two constitutive transcriptional units containing nucleotide sequences encoding the PdST6-like polypeptide (SEQ ID NO: 11) consisting of amino acid residues 108 to 497 of UniProt ID 066375 and having beta-galactoside alpha-2,6-sialyltransferase activity. In a next step, the novel strains were modified for growth on sucrose as in Example 1. The novel strains were evaluated in a growth experiment for production of sialic acid and 6′SL according to the culture conditions provided in Example 1 in which the strains were cultivated in minimal medium with 30 g/L sucrose and 20 g/L lactose. The strains were grown in four biological replicates in a 96-well plate. After 72 h of incubation, the culture broth was harvested, and the sugars were analyzed on UPLC. For each strain with a particular hydrolyzing UDP-N-acetylglucosamine-2-epimerase tested, the measured sialic acid and 6′SL concentrations were averaged over all biological replicates and then normalized to the averaged sialic acid and 6′SL concentration measured of the strain expressing the reference NeuC polypeptide from C. jejuni (UniProt ID Q93MP8). The experiment demonstrated that each strain with a particular transcriptional unit of a hydrolyzing UDP-N-acetylglucosamine-2-epimerase tested produced sialic acid and 6′SL. For example, the strain expressing SEQ ID NO:01 showed 122% 6′SL production relative to the strain expressing the reference NeuC from C. jejuni (UniProt ID Q93MP8).

### Example 3. Evaluation of Production of Sialic Acid and 6′SL with Modified E. coli Hosts when Evaluated in a Fed-Batch Fermentation Process with Sucrose and Lactose

The mutant E. coli strains as described in Example 2 were selected for further evaluation in fed-batch fermentation processes. Fed-batch fermentations at bioreactor scale were performed as described in Example 1. Sucrose was used as a carbon source and lactose was added in the batch medium. During fed-batch, sucrose was added via an additional feed. In contrast to the cultivation experiments that are described herein and wherein only end samples were taken at the end of cultivation (i.e., 72 hours as described herein), regular broth samples were taken at several time points during the fermentation process and the production of sialic acid and 6′SL was measured using UPLC as described in Example 1.

### Example 4. Evaluation of Production of Sialic Acid and LSTc with a Modified E. coli Host

The mutant E. coli strains as described in Example 2 are further modified with a genomic knock-in of a constitutive transcriptional unit containing nucleotide sequences encoding the galactoside beta-1,3-N-acetylglucosaminyltransferase LgtA from N. meningitidis (UniProt ID Q9JXQ6) and the N-acetylglucosamine beta-1,4-galactosyltransferase LgtB from N. meningitidis (UniProt ID Q51116, sequence version 02, 1 Dec. 2000) to allow production of lacto-N-neotetraose (LNnT, Gal-β1,4-GlcNAc-β1,3-Gal-β1,4-Glc). The novel strains are evaluated in a growth experiment for production of sialic acid and LSTc (Neu5Ac-α2,6-Gal-β1,4-GlcNAc-β1,3-Gal-β1,4-Glc) according to the culture conditions provided in Example 1 in which the strains are cultivated in minimal medium with 30 g/L sucrose and 20 g/L lactose. The strains are grown in four biological replicates in a 96-well plate. After 72 h of incubation, the culture broth is harvested, and production of sialic acid and LSTc is analyzed on UPLC.

### Example 5. Evaluation of Production of Sialic Acid and 6′SL with a Modified S. cerevisiae Host

A S. cerevisiae strain is modified for production of CMP-sialic acid and for expression of a sialyltransferase with a yeast expression plasmid containing the TRP1 selection marker and constitutive transcriptional units containing nucleotide sequences encoding the L-glutamine-D-fructose-6-phosphate aminotransferase glmS from E. coli (UniProt ID P17169, sequence version 04, 23 Jan. 2007), a hydrolyzing UDP-N-acetylglucosamine 2-epimerase selected from the list comprising SEQ ID NO:01, 02, 03, 04, 05, 06, 07 and 08, the N-acetylneuraminate synthase NeuB from N. meningitidis (UniProt ID E0NCD4), the N-acylneuraminate cytidylyltransferase NeuA from P. multocida (UniProt A0A849CI62) and the alpha-2,6-sialyltransferase PdST6 from P. damselae (UniProt ID 066375). The novel strains are evaluated in a growth experiment for production of sialic acid and 6′SL according to the culture conditions provided in Example 1, in which the appropriate selective medium comprises glucose as carbon source and lactose as precursors. The strains are grown in four biological replicates in a 96-well plate. After 72 h of incubation, the culture broth is harvested, and production of sialic acid and 6′SL is analyzed on UPLC.

### Example 6. Evaluation of Production of Sialic Acid and 3′SL with a Modified B. subtilis Host

In a first step, a B. subtilis strain is modified with genetic knockouts of the genes nagA, nagB and gamA and with genetic knock-ins of constitutive transcriptional units containing nucleotide sequences encoding the native fructose-6-P-aminotransferase glmS (UniProt ID P0C173), a hydrolyzing UDP-N-acetylglucosamine 2-epimerase selected from the list comprising SEQ ID NO:01, 02, 03, 04, 05, 06, 07 and 08, and the N-acetylneuraminate synthase NeuB from N. meningitidis (UniProt ID E0NCD4). In a next step, the strains are transformed with an expression plasmid containing a constitutive transcriptional unit containing nucleotide sequences encoding the N-acylneuraminate cytidylyltransferase neuA from P. multocida (UniProt ID A0A849CI62) and the alpha-2,3-sialyltransferasePmultST3 from Pasteurella multocida (UniProt ID Q9CLP3). The novel strains are evaluated in a growth experiment for production of sialic acid and 6′SL according to the culture conditions provided in Example 1, in which the appropriate selective medium comprises glucose as carbon source and lactose as precursors. The strain is grown in four biological replicates in a 96-well plate. After 72 h of incubation, the culture broth is harvested, and production of sialic acid and 3′SL is analyzed on UPLC.

### Example 7. Evaluation of Production of Sialic Acid and 3′SL with a Modified C. glutamicum Host

A mutant C. glutamicum strain is created by genetic knockouts of the genes nagA, nagB and gamA and by overexpressing the native fructose-6-P-aminotransferase glmS (UniProt ID Q8NND3, sequence version 03, 23 Jan. 2007), a hydrolyzing UDP-N-acetylglucosamine 2-epimerase selected from the list comprising SEQ ID NO:01, 02, 03, 04, 05, 06, 07 and 08, and the N-acetylneuraminate synthase NeuB from N. meningitidis (UniProt ID E0NCD4). In a next step, the strains are transformed with an expression plasmid containing a constitutive transcriptional unit containing nucleotide sequences encoding the N-acylneuraminate cytidylyltransferase neuA from P. multocida (UniProt ID A0A849CI62) and the alpha-2,3-sialyltransferase PmultST3 from P. multocida (UniProt ID Q9CLP3). The novel strains are evaluated in a growth experiment for production of sialic acid and 6′SL according to the culture conditions provided in Example 1, using MMsf medium comprising lactose. Regular samples are taken and evaluated via UPLC for production of sialic acid and 3′SL.

### Example 8. Evaluation of Production of 6′SL and LSTc with a Modified E. coli Host

An E. coli K-12 MG1655 strain was modified for production of LNT and LNnT comprising genomic knock-ins of constitutive transcriptional units containing nucleotide sequences encoding the galactoside beta-1,3-N-acetylglucosaminyltransferase lgtA from Neisseria meningitidis (UniProt ID Q9JXQ6), the beta-1,3-galactosyltransferase furA from Pseudogulbenkiania ferrooxidans (UniProt ID B9YZ84), the beta-1,3-galactosyltransferase wbdO from Salmonella enterica (UniProt ID Q5UHA8) and the beta-1,4-galactosyltransferase galT from Helicobacter pylori (UniProt ID Q9RHG8). To allow production of sialic acid, the strain was further modified with genomic knock-ins of constitutive transcriptional units containing nucleotide sequences encoding a hydrolyzing UDP-N-acetylglucosamine-2-epimerase selected from the list comprising SEQ ID NO:01, 02, 03, 04, 06, 08 and the reference polypeptide NeuC from Campylobacter jejuni (UniProt ID Q93MP8), and the N-acetylneuraminate synthase NeuB from N. meningitidis (UniProt ID E0NCD4). To allow production of 6′SL and LSTc, the strains were further modified with a plasmid containing a constitutive transcriptional unit containing nucleotide sequences encoding the N-acylneuraminate cytidylyltransferase neuA from C. jejuni (UniProt ID Q93MP7) and a constitutive transcriptional unit containing nucleotide sequences encoding the PdST6-like polypeptide (SEQ ID NO: 11) consisting of amino acid residues 108 to 497 of UniProt ID 066375 and having beta-galactoside alpha-2,6-sialyltransferase activity. In a next step, the novel strains were modified for growth on sucrose as in Example 1. The novel strains were evaluated in a growth experiment for production of 6′SL, LSTc, 6S(2)-LNnT (Neu5Ac-a2,6-(Gal-b1,4-GlcNAc-b1,3)-Gal-b1,4-Glc), 6S(2)-LNT (Neu5Ac-a2,6-(Gal-b1,3-GlcNAc-b1,3)-Gal-b1,4-Glc) and/or 6S(4)-LNT (Neu5Ac-a2,6-Gal-b1,3-GlcNAc-b1,3-Gal-b1,4-Glc) according to the culture conditions provided in Example 1 in which the strains were cultivated in minimal medium with 30 g/L sucrose and 20 g/L lactose. The strains were grown in four biological replicates in a 96-well plate. After 72 h of incubation, the culture broth was harvested, and the sugars were analyzed on UPLC. For each strain with a particular hydrolyzing UDP-N-acetylglucosamine-2-epimerase tested, the measured 6′SL and LSTc concentrations were averaged over all biological replicates and then normalized to the averaged 6′SL and LSTc concentration measured of the strain expressing the reference NeuC polypeptide from C. jejuni (UniProt ID Q93MP8). For each strain, the measured 6S(2)-LNnT, 6S(2)-LNT and/or 6S(4)-LNT concentrations were averaged over all biological replicates and then normalized to the averaged 6′SL concentration measured in that particular strain. The experiment demonstrated that each strain with a particular transcriptional unit of a hydrolyzing UDP-N-acetylglucosamine-2-epimerase tested produced 6′SL and LSTc. In addition, the data clearly showed that the production of 6′SL and LSTc could be tuned based on the hydrolyzing UDP-N-acetylglucosamine-2-epimerase selected (see Table 2). The data further showed small production of 6S(2)-LNT and 6S(4)-LNT in all samples, further comprising zero to small production of 6S(2)-LNnT (Results not shown). The data also showed that the production of 6S(2)-LNnT, 6S(2)-LNT and/or 6S(4)-LNT could be tuned based on the hydrolyzing UDP-N-acetylglucosamine-2-epimerase selected resulting in fine-tuning of the purity of 6′SL in the mixture comprising 6′SL and LSTc. For example, a higher purity for 6′SL in the mixture comprising 6′SL and LSTc could be reached when using an hydrolyzing UDP-N-acetylglucosamine-2-epimerase with SEQ ID NO:01, 02 or 06 (see Table 3).

TABLE 2
Relative production of 6′SL (%) and LSTc (%) in mutant E. coli strains A-F
expressing an alpha-2,6-sialyltransferase and a hydrolyzing UDP-N-
acetylglucosamine-2-epimerase and producing LNT, LNnT and
sialic acid, when evaluated in a growth experiment according
to the culture conditions provided in Example 1,
in which the cultivation medium contained 30 g/L sucrose and 20 g/L
lactose, and compared to a reference strain REFI with the same genetic
background but expressing NeuC from C. jejuni (UniProt ID Q93MP8).

| | Hydrolyzing UDP-N-acetylglucosamine-2- | | |
|---|---|---|---|
| Strain | epimerase expressed | 6′SL (%) | LSTc (%) |
| REF1 | NeuC (UniProt ID Q93MP8) | 100 | 100 |
| A | SEQ ID NO: 01 | 137 | 105 |
| B | SEQ ID NO: 02 | 81 | 75 |
| C | SEQ ID NO: 03 | 49 | 61 |
| D | SEQ ID NO: 04 | 67 | 70 |
| E | SEQ ID NO: 06 | 82 | 83 |
| F | SEQ ID NO: 08 | 22 | 40 |

TABLE 3
Relative production of 6′SL (%), 6S(2)-LNnT (%),
6S(2)-LNT (%) and/or 6S(4)-LNT (%) in mutant
E. coli strains A-F expressing an alpha-2,6-sialyltransferase and a
hydrolyzing UDP-N-acetylglucosamine-2-epimerase and producing
LNT, LNnT and sialic acid, when evaluated in a
growth experiment according to the culture conditions
provided in Example 1, in which the cultivation
medium contained 30 g/L sucrose and 20 g/L lactose, and
compared to a reference strain REF1 with the same genetic
background but expressing NeuC from C. jejuni (UniProt ID Q93MP8).

| | Hydrolyzing UDP-N- | | | 6S(2)- | |
|---|---|---|---|---|---|
| | acetylglucosamine-2- | 6′SL | | LNT | |
| Strain | epimerase expressed | (%) | 6S(2)-LNnT (%) | (%) | 6S(4)-LNT (%) |
| REF1 | NeuC (UniProt ID Q93MP8) | 100 | 6 | 46 | 67 |
| A | SEQ ID NO: 01 | 100 | 0 | 32 | 34 |
| B | SEQ ID NO: 02 | 100 | 0 | 46 | 53 |
| C | SEQ ID NO: 03 | 100 | 10 | 69 | 63 |
| D | SEQ ID NO: 04 | 100 | 17 | 53 | 65 |
| E | SEQ ID NO: 06 | 100 | 0 | 46 | 51 |
| F | SEQ ID NO: 08 | 100 | 111 | 103 | 62 |

### Example 9. Evaluation of Production of 3′SL, LSTa and LSTd with a Modified E. coli Host

An E. coli K-12 MG1655 strain was modified for production of LNT and LNnT comprising genomic knock-ins of constitutive transcriptional units containing nucleotide sequences encoding the galactoside beta-1,3-N-acetylglucosaminyltransferase lgtA from Neisseria meningitidis (UniProt ID Q9JXQ6), the beta-1,3-galactosyltransferase furA from Pseudogulbenkiania ferrooxidans (UniProt ID B9YZ84), the beta-1,3-galactosyltransferase wbdO from Salmonella enterica (UniProt ID Q5UHA8) and the beta-1,4-galactosyltransferase galT from Helicobacter pylori (UniProt ID Q9RHG8). To allow production of sialic acid, the strain was further modified with genomic knock-ins of constitutive transcriptional units containing nucleotide sequences encoding a hydrolyzing UDP-N-acetylglucosamine-2-epimerase selected from the list comprising SEQ ID NO:01, 02, 03, 04, 06, 08 and the reference polypeptide NeuC from Campylobacter jejuni (UniProt ID Q93MP8), and the N-acetylneuraminate synthase NeuB from N. meningitidis (UniProt ID E0NCD4). To allow production of 3′SL, LSTa and LSTd, the strains were further modified with a plasmid containing a constitutive transcriptional unit containing nucleotide sequences encoding neuA from C. jejuni (UniProt ID Q93MP7) and a constitutive transcriptional unit containing nucleotide sequences encoding the alpha-2,3-sialyltransferase PmultST3 from Pasteurella multocida (UniProt ID Q9CLP3). In a next step, the novel strains were modified for growth on sucrose as in Example 1. The novel strains were evaluated in a growth experiment for production of 3′SL, LSTa and LSTd according to the culture conditions provided in Example 1 in which the strains were cultivated in minimal medium with 30 g/L sucrose and 20 g/L lactose. The strains were grown in four biological replicates in a 96-well plate. After 72 h of incubation, the culture broth was harvested, and the sugars were analyzed on UPLC. For each strain with a particular hydrolyzing UDP-N-acetylglucosamine-2-epimerase tested, the measured 3′SL, LSTa and LSTd concentrations were averaged over all biological replicates and then normalized to the averaged 3′SL, LSTa and LSTd concentration measured of the strain expressing the reference NeuC polypeptide from C. jejuni (UniProt ID Q93MP8). The experiment demonstrated that each strain with a particular transcriptional unit of a hydrolyzing UDP-N-acetylglucosamine-2-epimerase tested produced 3′SL, LSTa and LSTd. In addition, the data clearly showed that the production of 3′SL, LSTa and LSTd could be tuned based on the hydrolyzing UDP-N-acetylglucosamine-2-epimerase selected (see Table 4).

TABLE 4
Relative production of 3′SL (%), LSTa (%) and LSTd (%) in
mutant E. coli strains G-L expressing an alpha-2,3-sialyltransferase
and a hydrolyzing UDP-N-acetylglucosamine-2-
epimerase and producing LNT, LNnT and sialic acid, when
evaluated in a growth experiment according to the culture
conditions provided in Example 1, in which the
cultivation medium contained 30 g/L sucrose
and 20 g/L lactose, and compared to a reference strain
REF2 with the same genetic background but
expressing NeuC from C. jejuni (UniProt ID Q93MP8).

| | Hydrolyzing UDP-N- | | | |
|---|---|---|---|---|
| | acetylglucosamine-2-epimerase | | | |
| Strain | expressed | 3′SL (%) | LSTa (%) | LSTd (%) |
| REF2 | NeuC (UniProt ID Q93MP8) | 100 | 100 | 100 |
| G | SEQ ID NO: 01 | 262 | 117 | 166 |
| H | SEQ ID NO: 02 | 112 | 96 | 98 |
| I | SEQ ID NO: 03 | 72 | 80 | 86 |
| J | SEQ ID NO: 04 | 79 | 71 | 75 |
| K | SEQ ID NO: 06 | 130 | 125 | 119 |
| L | SEQ ID NO: 08 | 44 | 46 | 50 |

### Example 10. Evaluation of Production of a Mixture of Sialylated and Non-Charged (Non-Sialylated) Oligosaccharides with a Modified E. coli Host

An E. coli K-12 MG1655 strain was modified for production of LNT and LNnT comprising genomic knock-ins of constitutive transcriptional units containing nucleotide sequences encoding the galactoside beta-1,3-N-acetylglucosaminyltransferase lgtA from Neisseria meningitidis (UniProt ID Q9JXQ6), the beta-1,3-galactosyltransferase furA from Pseudogulbenkiania ferrooxidans (UniProt ID B9YZ84), the beta-1,3-galactosyltransferase wbdO from Salmonella enterica (UniProt ID Q5UHA8) and the beta-1,4-galactosyltransferase galT from Helicobacter pylori (UniProt ID Q9RHG8). To allow production of sialic acid, the strain was further modified with genomic knock-ins of constitutive transcriptional units containing nucleotide sequences encoding a hydrolyzing UDP-N-acetylglucosamine-2-epimerase selected from the list comprising SEQ ID NO:04 and 06 and the reference polypeptide NeuC from Campylobacter jejuni (UniProt ID Q93MP8), and the N-acetylneuraminate synthase NeuB from N. meningitidis (UniProt ID E0NCD4). To allow production of sialylated oligosaccharides the strains were further modified with a genomic knock-in containing a constitutive transcriptional unit containing nucleotide sequences encoding neuA from P. multocida (UniProt ID A0A849CI62) and a constitutive transcriptional unit containing nucleotide sequences encoding the alpha-2,3-sialyltransferase PmultST3 from Pasteurella multocida (UniProt ID Q9CLP3). Next, to allow production of fucosylated oligosaccharides, the strains were modified with a genomic knock-in of constitutive transcriptional units for the phosphomannomutase manB from E. coli (UniProt ID P24175), the mannose-1-phosphate guanylyltransferase manC from E. coli (UniProt ID P24174, sequence version 03, 19 Jul. 2003), the GDP-mannose 4,6-dehydratase gmd from E. coli (UniProt ID P0AC88) and the GDP-L-fucose synthase fcl from E. coli (UniProt ID P32055, sequence version 02, 1 Nov. 1997). In addition, a plasmid containing constitutive transcriptional units containing nucleotide sequences encoding an alpha-1,2-fucosyltransferase (2FT) from Helicobacter sp. MIT 01-6242 (UniProt ID A0A1B1U4V1) and encoding an alpha-1,3-fucosyltransferase (3FT) from Porphyromonas catoniae (UniProt ID Z4WWI2). In a final step, the novel strains were modified for growth on sucrose as in Example 1. The novel strains were evaluated in a growth experiment for production of 2′FL, 3-FL, DiFL, 3′SL, LNT, LNnT, LSTa, LSTd, fucosylated LNT and/or fucosylated LNnT according to the culture conditions provided in Example 1 in which the strains were cultivated in minimal medium with 30 g/L sucrose and 20 g/L lactose. The strains were grown in four biological replicates in a 96-well plate. After 72 h of incubation, the culture broth was harvested, and the sugars were analyzed on UPLC. The experiment demonstrated that each strain with a particular transcriptional unit of a hydrolyzing UDP-N-acetylglucosamine-2-epimerase tested produced an oligosaccharide mixture comprising of 5 to 8% sialylated oligosaccharides and 95 to 92% non-charged (non-sialylated) oligosaccharides. Herein, the fraction of sialylated oligosaccharides comprised 3′SL, LSTa (Neu5Ac-α2,3-Gal-β1,3-GlcNAc-β1,3-Gal-β1,4-Glc) and LSTd (Neu5Ac-α2,3-Gal-β1,4-GlcNAc-β1,3-Gal-β1,4-Glc). The fraction of non-charged (non-sialylated) oligosaccharide comprised LNFP-I (Fuc-a1,2-Gal-b1,3-GlcNAc-b1,3-Gal-b1,4-Glc), LNnFP-I (Fuc-a1,2-Gal-b1,4-GlcNAc-b1,3-Gal-b1,4-Glc), LNDFH-I (Fuc-a1,4-(Fuc-a1,2-Gal-b1,3)-GlcNAc-b1,3-Gal-b1,4-Glc), Fuc-a1,2-Gal-b1,4-(Fuc-a1,3)-GlcNAc-b1,3-Gal-b1,4-Glc, Fuc-a1,2-Gal-b1,4-GlcNAc-b1,3-Gal-b1,4-(Fuc-a1,3)-Glc, 2′FL, DiFL, LNnT and LNT. In addition, the data showed that the production of sialylated oligosaccharides and non-charged (non-sialylated) oligosaccharides could be tuned based on the hydrolyzing UDP-N-acetylglucosamine-2-epimerase selected (see Table 5).

TABLE 5
Production of a mixture comprising sialylated oligosaccharides
(%) and non-charged (non-sialylated) oligosaccharides (%) in mutant
E. coli strains M and N expressing an alpha-1,2-fucosyltransferase, an alpha-
1,3-fucosyltransferase, an alpha-2,3-sialyltransferase and a hydrolyzing UDP-
N-acetylglucosamine-2-epimerase and producing LNT, LNnT, sialic
acid, and GDP-fucose when evaluated in a growth experiment according to the
culture conditions provided in Example 1, in which the cultivation medium contained
30 g/L sucrose and 20 g/L lactose, and compared to a reference strain REF2 with the
same genetic background but expressing NeuC from C. jejuni (UniProt ID Q93MP8).

| | Hydrolyzing UDP-N- | Sialylated | Non-charged (non- |
|---|---|---|---|
| | acetylglucosamine-2-epimerase | oligosaccharides | sialylated |
| Strain | expressed | (%) | oligosaccharides) (%) |
| REF3 | NeuC (UniProt ID Q93MP8) | 7 | 93 |
| M | SEQ ID NO: 04 | 8 | 92 |
| N | SEQ ID NO: 06 | 5 | 95 |

1.-34. (canceled)

35. A cell comprising a pathway for producing a negatively charged oligosaccharide, wherein the cell is genetically engineered to possess, express, and/or overexpress a hydrolyzing UDP-N-acetyl-D-glucosamine-2-epimerase wherein the hydrolyzing UDP-N-acetyl-D-glucosamine-2-epimerase comprises an amino acid sequence comprising a conserved motif with SEQ ID NO: 12 and having hydrolyzing UDP-N-acetyl-D-glucosamine-2-epimerase activity.

36. The cell of claim 35, wherein the hydrolyzing UDP-N-acetyl-D-glucosamine-2-epimerase comprises an amino acid sequence that is at least 80% identical over a stretch of at least 150 amino acid residues and/or at least 200 amino acid residues, to any one of the amino acid sequences as represented by SEQ ID NO: 1, 2, 3, 4, 5, 6, 7, or 8.

37. The cell of claim 35, wherein the hydrolyzing UDP-N-acetyl-D-glucosamine-2-epimerase comprises an amino acid sequence:
comprising an IPR domain selected from the group consisting of IPR003331, IPR020004 and IPR029767 as defined by InterPro 90.0 as released on 4 Aug. 2022,
comprising a PF02350 motif as defined by PFAM 32.0 as released in September 2018,
comprising a cd03786 motif as defined by CDD v3.17 as released in September 2016,
comprising a panther domain selected from the group consisting of PTHR43174 and PTHR43174:SF3 as defined by PANTHER 17.0 as released on 23 Feb. 2022,
that is at least 80% identical to any one of the amino acid sequences as represented by SEQ ID NOs: 1, 2, 3, 4, 5, 6, 7, or 8 over a stretch of at least 150 amino acid residues,
that is at least 80% identical to any one of the full-length amino acid sequences as represented by SEQ ID NOs: 1, 2, 3, 4, 5, 6, 7, or 8, and/or
as represented by any one of SEQ ID NOs: 1, 2, 3, 4, 5, 6, 7, or 8.

38. The cell of claim 35, wherein the hydrolyzing UDP-N-acetyl-D-glucosamine-2-epimerase is a nnaA polypeptide.

39. The cell of claim 35, wherein the cell contains a nucleic acid molecule, which comprises a polynucleotide that encodes the hydrolyzing UDP-N-acetyl-D-glucosamine-2-epimerase.

40. The cell of claim 39, wherein the nucleic acid molecule is: operably linked to control sequences recognized by the cell, the nucleic acid molecule further i)
being integrated in the genome of the cell and/or ii) presented to the cell on a vector, and/or foreign to the cell.

41. The cell of claim 35, wherein the pathway for producing the negatively charged oligosaccharide is a sialylation pathway, and/or
the cell is genetically engineered to comprise a sialylation pathway, and/or
the cell comprises a sialylation pathway wherein the sialylation pathway has been genetically engineered.

42. The cell of claim 35, wherein the cell:
further comprises a pathway selected from the group consisting of fucosylation pathway, galactosylation pathway, N-acetylglucosaminylation pathway, N-acetylgalactosaminylation pathway, mannosylation pathway and N-acetylmannosaminylation pathway,
is genetically engineered to comprise at least one pathway selected from the group consisting of fucosylation pathway, galactosylation pathway, N-acetylglucosaminylation pathway, N-acetylgalactosaminylation pathway, mannosylation pathway and N-acetylmannosaminylation pathway,
comprises at least one pathway selected from the group consisting of fucosylation pathway, galactosylation pathway, N-acetylglucosaminylation pathway, N-acetylgalactosaminylation pathway, mannosylation pathway and N-acetylmannosaminylation pathway wherein at least one of the pathway(s) has/have been genetically engineered,
possesses, expresses and/or overexpresses one or more glycosyltransferase(s) selected from the group consisting of fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
is capable of producing and/or produces one or more nucleotide-activated sugars,
is genetically engineered for producing one or more of the nucleotide-activated sugar(s),
comprises a pathway for the synthesis of a nucleotide-activated sugar selected from the group consisting of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), GDP-fucose; (GDP-Fuc), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid, CMP-Neu5Ac, CMP-Neu4Ac, CMP-Neu5Ac9N3, CMP-Neu4,5Ac2, CMP-Neu5,7Ac2, CMP-Neu5,9Ac2, CMP-Neu5,7(8,9)Ac2, CMP-N-glycolylneuraminic acid (CMP-Neu5Gc), CMP-2-keto-3-deoxymanno-octulonic acid (CMP-KDO), GDP-rhamnose and UDP-xylose,
possesses, expresses and/or overexpresses one or more genes selected from the group consisting of mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, L-glutamine D-fructose-6-phosphate aminotransferase, phosphoglucosamine mutase, N-acetylglucosamine-6-P deacetylase, N-acylglucosamine 2-epimerase, UDP-N-acetylglucosamine 2-epimerase, N-acetylmannosamine-6-phosphate 2-epimerase, UDP-GlcNAc 2-epimerase/kinase, glucosamine 6-phosphate N-acetyltransferase, N-acetylglucosamine-6-phosphate phosphatase, phosphoacetylglucosamine mutase, N-acetylglucosamine 1-phosphate uridylyltransferase, glucosamine-1-phosphate acetyltransferase, bifunctional N-acetylglucosamine-1-phosphate uridyltransferase/glucosamine-1-phosphate acetyltransferase, sialic acid synthase, N-acetylneuraminate lyase, N-acylneuraminate-9-phosphate synthase, N-acylneuraminate-9-phosphatase, sialic acid transporter, CMP kinase, CMP-sialic acid synthase, d-arabinose 5-phosphate isomerase, KDO-8P synthase, KDO 8-phosphate phosphatase, CMP-KDO synthetase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase,
is capable of producing and/or produces N-acetylmannosamine (ManNAc), a sialic acid residue and/or N-acetylglucosamine (GlcNAc), wherein the sialic acid residue is selected from the group consisting of Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4; Neu4,5,7,8,9Ac5; Neu5Gc and 2-keto-3-deoxymanno-octulonic acid (KDO),
is genetically engineered for producing ManNAc, a sialic acid residue and/or GlcNAc,
is capable of producing and/or produces the negatively charged oligosaccharide from one or more precursor(s),
is capable of producing and/or produces the negatively charged oligosaccharide from lactose,
is capable of producing and/or produces at least one or more precursor(s) that is/are used to produce the negatively charged oligosaccharide,
is capable of producing and/or produces all precursors that are used to produce the negatively charged oligosaccharide,
is genetically engineered for producing at least one or more precursor(s) that is/are used to produce the negatively charged oligosaccharide,
is capable of producing and/or produces lactose,
is genetically engineered for producing all precursors that are used to produce the negatively charged oligosaccharide,
is capable of producing and/or produces phosphoenolpyruvate (PEP),
is modified for enhanced synthesis and/or supply of PEP compared to a non-modified progenitor,
comprises a modification for reduced production of acetate compared to a non-modified progenitor,
possesses, expresses and/or overexpresses at least one gene selected from the group consisting of genes encoding a disulfide bond isomerase, a thiol oxidase or a chaperone,
comprises a catabolic pathway for selected mono-, di- or oligosaccharides, which is at least partially inactivated, the mono-, di-, or oligosaccharides being involved in and/or required for the synthesis of the negatively charged oligosaccharide, and/or
possesses, expresses and/or overexpresses a membrane transporter protein.

43. The cell of claim 42, wherein at least one of the one or more precursor(s) is internalized in the cell via one or more membrane transporter protein(s).

44. The cell of claim 35, wherein one or more gene(s) involved in one or more reductive pathway(s) in the cell is/are rendered less functional or is/are knocked-out.

45. The cell of claim 44, wherein the one or more gene(s) involved in one or more reductive pathway(s) is/are rendered less functional by insertion, deletion and/or modification of one or more nucleotide(s) in one or more polynucleotide sequence(s) selected from the group consisting of promoter sequence, ribosome binding site, untranslated region, coding sequence and transcription terminator sequence of the one or more gene(s).

46. The cell of claim 35, wherein the negatively charged oligosaccharide is:
a sialylated oligosaccharide having at least one sialic acid residue selected from the group consisting of Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4; Neu4,5,7,8,9Ac5; Neu5Gc and 2-keto-3-deoxymanno-octulonic acid (KDO),
an oligosaccharide selected from the group consisting of a negatively charged milk oligosaccharide, a sialylated milk oligosaccharide, a negatively charged mammalian milk oligosaccharide (MMO), a sialylated MMO, a negatively charged human milk oligosaccharide (HMO), a sialylated HMO, O-antigen, the oligosaccharide repeats present in capsular polysaccharides, peptidoglycan, an amino-sugar, Lewis-type antigen oligosaccharide, a negatively charged animal oligosaccharide, a sialylated animal oligosaccharide, a negatively charged animal oligosaccharide selected from the group consisting of N-glycans and O-glycans, a sialylated animal oligosaccharide selected from the group consisting of N-glycans and O-glycans, a negatively charged plant oligosaccharide, a sialylated plant oligosaccharide, a negatively charged plant oligosaccharide selected from the group consisting of N-glycans and O-glycans, a sialylated plant oligosaccharide selected from the group consisting of N-glycans and O-glycans, sialylated oligosaccharide, N-acetyllactosamine containing negatively charged oligosaccharide, N-acetyllactosamine containing sialylated oligosaccharide, lacto-N-biose containing negatively charged oligosaccharide, lacto-N-biose containing sialylated oligosaccharide, and/or
selected from the group consisting of 3′sialyllactose (3′SL), 6′sialyllactose (6′SL), 3′-sialyllactosamine, 3,6-disialyllactose, 8,3-disialyllactose, sialylated lacto-N-triose, sialyllacto-N-tetraose a (LSTa), KDOα-2,3Galβ-1,3GlcNAcβ-1,3Galβ-1,4Glc, sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose, Neu5Ac-α2,3-Gal-β-1,4-GlcNAc-β-1,3-Gal, Neu5Ac-α2,3-Gal-β-1,3-GlcNAc-β-1,3-Gal, 3′-KDO-lactose, 3′-KDO-lactosamine, 3′-KDO-lacto-N-biose, 3′-KDO-6′sialyllactose, 3′KDO-8-sialyllactose, KDO-2,3Galβ-1,3GalNacβ-1,3Galα-1,4Galβ-1,4Gal, KDOα-2,3Galβ-1,4GlcNacβ-1,3Galβ-1,4Glc, 3′-KDO-3-fucosyllactose, Neu5Ac-a2,8-Neu5Ac-α2,3-Gal-β-1,3-GlcNAc-β-1,3-Gal, 3′-Sialyl-2′-fucosyllactose, 6′-Sialyl-2′-fucosyllactose, 6′-Sialyl-3-fucosyllactose, Neu5Ac-α2,6-(Neu5Ac-a2,3-)Gal-β-1,4-Glc, 3′-Sialyl-3-fucosyllactosamine, Fuc-a1,4-(Neu5Ac-α2,3-Gal-β-1,3-)GlcNAc, 6′-Sialyllacto-N-biose, 3′-Sialyllacto-N-biose, Neu5Ac-α2,6-(GlcNAc-β-1,3-)Gal-β-1,4-Glc, Neu5Ac-α2,6-(Gal-β-1,4-(Fuc-a1,3-)GlcNAc-β-1,3-)Gal-β-1,4-Glc, Neu5Ac-α2,3-Gal-β-1,4-(Fuc-a1,3-)GlcNAc-β-1,3-Gal-β-1,4-Glc, Neu5Ac-α2,6-(Neu5Ac-α2,6-Gal-β-1,4-(Fuc-a1,3-)GlcNAc-β-1,3-)Gal-β-1,4-Glc, Neu5Ac-α2,6-(Gal-β-1,4-GlcNAc-β-1,3-)Gal-β-1,4-Glc, Neu5Ac-α2,6-(Gal-β-1,3-GlcNAc-β-1,3-)Gal-β-1,4-(Fuc-a1,3-)Glc, Neu5Ac-α2,6-Gal-β-1,3-GlcNAc-β-1,3-Gal-β-1,4-(Fuc-a1,3-)Glc, Neu5Ac-α2,3-Gal-β-1,3-GlcNAc-β-1,3-Gal-β-1,4-(Fuc-a1,3-)Glc, Neu5Ac-a2,3-(Fuc-a1,2-)Gal-β-1,3-GlcNAc-β-1,3-Gal-β-1,4-Glc, Neu5Ac-α2,6-(Fuc-a1,2-Gal-β-1,3-GlcNAc-β-1,3-)Gal-β-1,4-Glc, Neu5Ac-α2,6-(Fuc-a1,2-)Gal-β-1,3-GlcNAc-β-1,3-Gal-β-1,4-Glc, Fuc-α1,4-(Neu5Ac-α2,3-Gal-β-1,3-)GlcNAc-β-1,3-Gal-β-1,4-Glc, Neu5Ac-α2,6-(Neu5Ac-α2,6-Gal-β-1,3-GlcNAc-β-1,3-)Gal-β-1,4-(Fuc-α1,3-)Glc, Neu5Ac-α2,6-(Neu5Ac-α2,6-(Fuc-α1,2-)Gal-β-1,3-GlcNAc-β-1,3-)Gal-β-1,4-Glc, Neu5Ac-α2,6-(Gal-β-1,3-GlcNAc-β-1,3-)Gal-β-1,4-Glc, Neu5Ac-α2,6-Gal-β-1,3-GlcNAc-β-1,3-Gal-β-1,4-Glc, Neu5Ac-α2,6-(Neu5Ac-a2,3)-Gal-β-1,4-GlcNAc-β-1,3-Gal-β-1,4-Glc, Neu5Ac-α2,6-(Neu5Ac-α2,3-Gal-β-1,3-GlcNAc-β-1,3-)Gal-β-1,4-Glc, Neu5Ac-α2,6-(Neu5Ac-α2,6-Gal-β-1,3-GlcNAc-β-1,3)-Gal-β-1,4-Glc, Neu5Ac-α2,6-(Neu5Ac-α2,6-(Neu5Ac-a2,3)-Gal-β-1,3-GlcNAc-β-1,3)-Gal-β-1,4-Glc, Neu5Ac-α2,6-(Neu5Ac-α2,3-Gal-β-1,4-GlcNAc-β-1,3)-Gal-β-1,4-Glc, Neu5Ac-α2,6-(Neu5Ac-α2,6-Gal-β-1,4-GlcNAc-β-1,3)-Gal-β-1,4-Glc, Neu5Ac-α2,6-(Neu5Ac-α2,6-(Neu5Ac-a2,3)-Gal-β-1,4-GlcNAc-β-1,3)-Gal-β-1,4-Glc, Neu5Ac-α2,6-(Gal-β-1,4-GlcNAc-β-1,3-Gal-β-1,4-GlcNAc-β-1,3)-Gal-β-1,4-Glc, Neu5Ac-α2,6-(Gal-β-1,4-GlcNAc-β-1,3)-Gal-β-1,4-GlcNAc-β-1,3-Gal-β-1,4-Glc, Neu5Ac-α2,6-Gal-β-1,4-GlcNAc-β-1,3-Gal-β-1,4-GlcNAc-β-1,3-Gal-β-1,4-Glc, Neu5Ac-α2,6-(Neu5Ac-α2,6-(Neu5Ac-α2,6-Gal-β-1,4-GlcNAc-β-1,3)-Gal-β-1,4-GlcNAc-β-1,3)-Gal-β-1,4-Glc, Neu5Ac-α2,3-Gal-β-1,4-GlcNAc-β-1,3-Gal-β-1,4-GlcNAc-β-1,3-Gal-β-1,4-Glc, 6′-KDO-lactose, 6′-KDO-lactosamine, 6′-KDO-lacto-N-biose, KDO-lacto-N-triose, KDO-lacto-N-tetraose, KDO-lacto-N-tetraose, and combinations thereof.

47. The cell of claim 35, wherein the cell is selected from the group consisting of prokaryotic cells and eukaryotic cells.

48. The cell of claim 35, wherein the cell produces 30 g/L or more of the negatively charged oligosaccharide in the whole broth and/or supernatant and/or wherein the negatively charged oligosaccharide in the whole broth and/or supernatant has a purity of at least 80% measured on the total amount of the negatively charged oligosaccharide and its precursor(s) produced by the cell in the whole broth and/or supernatant, respectively.

49. The cell of claim 35, wherein the cell is stably cultured in a cultivation or incubation medium.

50. The cell of claim 35, wherein the cell is capable of producing and/or produces a mixture of two or more oligosaccharides wherein at least one of the two or more oligosaccharides is the negatively charged oligosaccharide.

51. The cell of claim 35, wherein the cell is capable of producing and/or produces a mixture of one or more disaccharide(s) and one or more oligosaccharide(s) wherein at least one of the one or more oligosaccharide(s) is the negatively charged oligosaccharide.

52. The cell of claim 35, wherein the cell is capable of producing and/or produces a mixture comprising the negatively charged oligosaccharide and at least one non-charged (neutral) oligosaccharide.

53. The cell of claim 35, wherein the cell is capable of producing and/or produces a mixture comprising 1) the negatively charged oligosaccharide and 2) a disaccharide and/or a non-charged (neutral) oligosaccharide.

54. A method of producing a negatively charged oligosaccharide, the method comprising:
i. cultivating and/or incubating the cell of claim 35 in cultivation and/or incubation medium under conditions permissive to produce the negatively charged oligosaccharide, and
ii. optionally, followed by separation and/or purification of the negatively charged oligosaccharide from the cultivation and/or incubation.

55. The method according to claim 54, wherein the negatively charged oligosaccharide is:
a sialylated oligosaccharide having at least one sialic acid residue selected from the group consisting of Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4; Neu4,5,7,8,9Ac5; Neu5Gc and 2-keto-3-deoxymanno-octulonic acid (KDO),
an oligosaccharide selected from the group consisting of a negatively charged milk oligosaccharide, a sialylated milk oligosaccharide, a negatively charged mammalian milk oligosaccharide (MMO), a sialylated MMO, a negatively charged human milk oligosaccharide (HMO), a sialylated HMO, O-antigen, the oligosaccharide repeats present in capsular polysaccharides, peptidoglycan, an amino-sugar, Lewis-type antigen oligosaccharide, a negatively charged animal oligosaccharide, a sialylated animal oligosaccharide, a negatively charged animal oligosaccharide selected from the group consisting of N-glycans and O-glycans, a sialylated animal oligosaccharide selected from the group consisting of N-glycans and O-glycans, a negatively charged plant oligosaccharide, a sialylated plant oligosaccharide, a negatively charged plant oligosaccharide selected from the group consisting of N-glycans and O-glycans, a sialylated plant oligosaccharide selected from the group consisting of N-glycans and O-glycans, sialylated oligosaccharide, N-acetyllactosamine containing negatively charged oligosaccharide, N-acetyllactosamine containing sialylated oligosaccharide, lacto-N-biose containing negatively charged oligosaccharide, lacto-N-biose containing sialylated oligosaccharide, and/or
selected from the group consisting of 3′sialyllactose (3′SL), 6′sialyllactose (6′SL), 3′-sialyllactosamine, 3,6-disialyllactose, 8,3-disialyllactose, sialylated lacto-N-triose, sialyllacto-N-tetraose a (LSTa), KDOα-2,3Galβ-1,3GlcNAcβ-1,3Galβ-1,4Glc, sialyllacto-N-tetraose b (LSTb), sialyllacto-N-tetraose c (LSTc), sialyllacto-N-tetraose d (LSTd), disialyllacto-N-tetraose, disialyllacto-N-neotetraose, monosialyllacto-N-hexaose, disialyllacto-N-hexaose I, disialyllacto-N-hexaose II, monosialyllacto-N-neohexaose I, monosialyllacto-N-neohexaose II, disialyllacto-N-neohexaose, 3′-sialyl-3-fucosyllactose, fucodisialyllacto-N-hexaose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose, Neu5Ac-α2,3-Gal-β-1,4-GlcNAc-β-1,3-Gal, Neu5Ac-α2,3-Gal-β-1,3-GlcNAc-β-1,3-Gal, 3′-KDO-lactose, 3′-KDO-lactosamine, 3′-KDO-lacto-N-biose, 3′-KDO-6′sialyllactose, 3′KDO-8-sialyllactose, KDO-2,3Galβ-1,3GalNaco-1,3Galα-1,4Galβ-1,4Gal, KDOα-2,3Galβ-1,4GlcNacβ-1,3Galβ-1,4Glc, 3′-KDO-3-fucosyllactose, Neu5Ac-α2,8-Neu5Ac-α2,3-Gal-β-1,3-GlcNAc-β-1,3-Gal, 3′-Sialyl-2′-fucosyllactose, 6′-sialyl-2′-fucosyllactose, 6′-Sialyl-3-fucosyllactose, Neu5Ac-α2,6-(Neu5Ac-a2,3-)Gal-β-1,4-Glc, 3′-Sialyl-3-fucosyllactosamine, Fuc-α1,4-(Neu5Ac-α2,3-Gal-β-1,3-)GlcNAc, 6′-sialyllacto-N-biose, 3′-sialyllacto-N-biose, Neu5Ac-α2,6-(GlcNAc-β-1,3-)Gal-β-1,4-Glc, Neu5Ac-α2,6-(Gal-β-1,4-(Fuc-α1,3-)GlcNAc-β-1,3-)Gal-β-1,4-Glc, Neu5Ac-α2,3-Gal-β-1,4-(Fuc-α1,3-)GlcNAc-β-1,3-Gal-β-1,4-Glc, Neu5Ac-α2,6-(Neu5Ac-α2,6-Gal-β-1,4-(Fuc-α1,3-)GlcNAc-β-1,3-)Gal-β-1,4-Glc, Neu5Ac-α2,6-(Gal-β-1,4-GlcNAc-β-1,3-)Gal-β-1,4-Glc, Neu5Ac-α2,6-(Gal-β-1,3-GlcNAc-β-1,3-)Gal-β-1,4-(Fuc-α1,3-)Glc, Neu5Ac-α2,6-Gal-β-1,3-GlcNAc-β-1,3-Gal-β-1,4-(Fuc-α1,3-)Glc, Neu5Ac-α2,3-Gal-β-1,3-GlcNAc-β-1,3-Gal-β-1,4-(Fuc-α1,3-)Glc, Neu5Ac-a2,3-(Fuc-α1,2-)Gal-β-1,3-GlcNAc-β-1,3-Gal-β-1,4-Glc, Neu5Ac-α2,6-(Fuc-α1,2-Gal-β-1,3-GlcNAc-β-1,3-)Gal-β-1,4-Glc, Neu5Ac-α2,6-(Fuc-α1,2-)Gal-β-1,3-GlcNAc-β-1,3-Gal-β-1,4-Glc, Fuc-α1,4-(Neu5Ac-α2,3-Gal-β-1,3-)GlcNAc-β-1,3-Gal-β-1,4-Glc, Neu5Ac-α2,6-(Neu5Ac-α2,6-Gal-β-1,3-GlcNAc-β-1,3-)Gal-β-1,4-(Fuc-α1,3-)Glc, Neu5Ac-α2,6-(Neu5Ac-α2,6-(Fuc-α1,2-)Gal-β-1,3-GlcNAc-β-1,3-)Gal-β-1,4-Glc, Neu5Ac-α2,6-(Gal-β-1,3-GlcNAc-β-1,3-)Gal-β-1,4-Glc, Neu5Ac-α2,6-Gal-β-1,3-GlcNAc-β-1,3-Gal-β-1,4-Glc, Neu5Ac-α2,6-(Neu5Ac-a2,3)-Gal-β-1,4-GlcNAc-β-1,3-Gal-β-1,4-Glc, Neu5Ac-α2,6-(Neu5Ac-α2,3-Gal-β-1,3-GlcNAc-β-1,3-)Gal-β-1,4-Glc, Neu5Ac-α2,6-(Neu5Ac-α2,6-Gal-β-1,3-GlcNAc-β-1,3)-Gal-β-1,4-Glc, Neu5Ac-α2,6-(Neu5Ac-α2,6-(Neu5Ac-a2,3)-Gal-β-1,3-GlcNAc-β-1,3)-Gal-β-1,4-Glc, Neu5Ac-α2,6-(Neu5Ac-α2,3-Gal-β-1,4-GlcNAc-β-1,3)-Gal-β-1,4-Glc, Neu5Ac-α2,6-(Neu5Ac-α2,6-Gal-β-1,4-GlcNAc-β-1,3)-Gal-β-1,4-Glc, Neu5Ac-α2,6-(Neu5Ac-α2,6-(Neu5Ac-a2,3)-Gal-β-1,4-GlcNAc-β-1,3)-Gal-β-1,4-Glc, Neu5Ac-α2,6-(Gal-β-1,4-GlcNAc-β-1,3-Gal-β-1,4-GlcNAc-β-1,3)-Gal-β-1,4-Glc, Neu5Ac-α2,6-(Gal-β-1,4-GlcNAc-β-1,3)-Gal-β-1,4-GlcNAc-β-1,3-Gal-β-1,4-Glc, Neu5Ac-α2,6-Gal-β-1,4-GlcNAc-β-1,3-Gal-β-1,4-GlcNAc-β-1,3-Gal-β-1,4-Glc, Neu5Ac-α2,6-(Neu5Ac-α2,6-(Neu5Ac-α2,6-Gal-β-1,4-GlcNAc-β-1,3)-Gal-β-1,4-GlcNAc-β-1,3)-Gal-β-1,4-Glc, Neu5Ac-α2,3-Gal-β-1,4-GlcNAc-β-1,3-Gal-β-1,4-GlcNAc-β-1,3-Gal-β-1,4-Glc, 6′-KDO-lactose, 6′-KDO-lactosamine, 6′-KDO-lacto-N-biose, KDO-lacto-N-triose, KDO-lacto-N-tetraose, KDO-lacto-N-tetraose, and combinations thereof.

56. The method according to claim 54, wherein the cultivation or incubation medium:
comprises one or more precursor(s) that is/are used for producing the negatively charged oligosaccharide,
contains at least one carbon source selected from the group consisting of a monosaccharide; disaccharide; oligosaccharide; polysaccharide; polyol; glycerol; a complex medium including molasses, corn steep liquor, peptone, tryptone or yeast extract; glucose; glycerol; fructose; sucrose; maltose; lactose; arabinose; maltooligosaccharides; maltotriose; sorbitol; xylose; rhamnose; galactose; mannose; methanol; ethanol; trehalose; starch; cellulose; hemi-cellulose; molasses; corn-steep liquor; high-fructose syrup; acetate; citrate; lactate and pyruvate,
contains at least one compound selected from the group consisting of lactose, galactose, glucose, UDP-galactose (UDP-Gal), sialic acid, CMP-sialic acid, CMP-Neu5Ac and CMP-KDO,
is a chemically defined medium, and/or
is a minimal salt medium comprising sulphate, phosphate, chloride, ammonium, calcium, magnesium, sodium, potassium, iron, copper, zinc, manganese cobalt and/or selenium.

57. The method according to claim 54, wherein the method comprises at least one of the following steps:
i) utilizing a cultivation or incubation medium comprising at least one precursor and/or acceptor;
ii) adding to the cultivation or incubation medium in a reactor or incubator at least one precursor and/or acceptor feed wherein the total reactor or incubator volume ranges from 250 mL to 10,000 m³ (cubic meter);
iii) adding to the cultivation or incubation medium in a reactor or incubator at least one precursor and/or acceptor feed wherein the total reactor or incubator volume ranges from 250 mL to 10,000 m³, in a continuous manner, and so that the final volume of the cultivation or incubation medium is not more than three-fold, not more than two-fold and/or less than two-fold of the volume of the cultivation or incubation medium before the addition of the precursor and/or acceptor feed;
iv) adding to the cultivation or incubation medium in a reactor or incubator at least one precursor and/or acceptor feed wherein the total reactor or incubator volume ranges from 250 mL to 10,000 m², in a continuous manner, and so that the final volume of the cultivation or incubation medium is not more than three-fold, not more than two-fold and/or less than two-fold of the volume of the cultivation or incubation medium before the addition of the precursor and/or acceptor feed and wherein the pH of the precursor and/or acceptor feed is set between 2.0 and 10.0 and/or the temperature of the precursor and/or acceptor feed is kept between 20° C. and 80° C.;
v) adding at least one precursor and/or acceptor feed in a continuous manner to the cultivation or incubation medium over the course of 1 day, 2 days, 3 days, 4 days or 5 days via a precursor and/or acceptor feeding solution;
vi) adding at least one precursor and/or acceptor feed in a continuous manner to the cultivation or incubation medium over the course of 1 day, 2 days, 3 days 4 days or 5 days via a precursor and/or acceptor feeding solution and wherein the concentration of the precursor and/or acceptor feeding solution is 50 g/L, 75 g/L, 100 g/L, 125 g/L, 150 g/L, 175 g/L, 200 g/L, 225 g/L, 250 g/L, 275 g/L, 300 g/L, 325 g/L, 350 g/L, 375 g/L, 400 g/L, 450 g/L, 500 g/L, 550 g/L or 600 g/L; and/or wherein the pH of the precursor and/or acceptor feeding solution is set between 2.0 and 10.0 and/or wherein the temperature of the precursor and/or acceptor feeding solution is kept between 20° C. and 80° C.;
the method resulting in the negatively charged oligosaccharide with a concentration of at least 50 g/L in the final volume of the cultivation or incubation.

58. The method according to claim 54, wherein the method comprises at least one of the following steps:
i) utilizing a cultivation or incubation medium comprising at least 50 grams of precursor per liter of initial reactor or incubator volume wherein the reactor or incubator volume ranges from 250 mL to 10,000 m3 (cubic meter);
ii) utilizing a cultivation or incubation medium comprising at least 5095 grams of acceptor per liter of initial reactor or incubator volume wherein the reactor or incubator volume ranges from 250 mL to 10,000 m3 (cubic meter);
iii) adding to the cultivation or incubation medium in a reactor or incubator a precursor feed comprising at least 50 grams of precursor per liter of initial reactor or incubator volume, wherein the reactor or incubator volume ranges from 250 mL to 10,000 m3 (cubic meter), in a continuous manner and so that the final volume of the cultivation or incubation medium is not more than three-fold, not more than two-fold and/or less than two-fold of the volume of the cultivation or incubation medium before the addition of the precursor feed;
iv) adding to the cultivation or incubation medium in a reactor or incubator an acceptor feed comprising at least 50 grams of acceptor per liter of initial reactor or incubator volume wherein the reactor or incubator volume ranges from 250 mL to 10,000 m3 (cubic meter), in a continuous manner and so that the final volume of the cultivation or incubation medium is not more than three-fold, not more than two-fold and/or less than two-fold of the volume of the cultivation or incubation medium before the addition of the acceptor feed;
v) adding to the cultivation or incubation medium a precursor feed comprising at least 50450 grams of precursor per liter of initial reactor or incubator volume wherein the total reactor or incubator volume ranges from 250 mL to 10,000 m3 (cubic meter), in a continuous manner and so that the final volume of the cultivation or incubation medium is not more than three-fold, not more than two-fold and/or less than two-fold of the volume of the cultivation or incubation medium before the addition of the precursor feed and wherein the pH of the precursor feed is set between 2.0 and 10.0 and/or the temperature of the precursor feed is kept between 20° C. and 80° C.;
vi) adding to the cultivation or incubation medium an acceptor feed comprising at least 50 grams of acceptor per liter of initial reactor or incubator volume wherein the total reactor or incubator volume ranges from 250 mL to 10,000 m3 (cubic meter), in a continuous manner so that the final volume of the cultivation or incubation medium is not more than three-fold, not more than two-fold and/or less than two-fold of the volume of the cultivation or incubation medium before the addition of the acceptor feed and wherein the pH of the acceptor feed is set between 2.0 and 10.0 and/or the temperature of the acceptor feed is kept between 20° C. and 80° C.;
vii) adding a precursor and/or acceptor feed in a continuous manner to the cultivation or incubation medium over the course of 1 day, 2 days 3 days, 4 days or 5 days via a precursor and/or acceptor feeding solution;
viii) adding a precursor feed in a continuous manner to the cultivation or incubation medium over the course of 1 day, 2 days, 3 days, 4 days or 5 days via a precursor feeding solution and wherein the concentration of the precursor feeding solution is 50 g/L, 75 g/L, 100 g/L, 125 g/L, 150 g/L, 175 g/L, 200 g/L, 225 g/L, 250 g/L, 275 g/L, 300 g/L, 325 g/L, 350 g/L, 375 g/L, 400 g/L, 450 g/L, 500 g/L, 550 g/L or 600 g/L;
ix) adding an acceptor feed in a continuous manner to the cultivation or incubation medium over the course of 1 day 2 days 3 days 4 days or 5 days via an acceptor feeding solution and wherein the concentration of the acceptor feeding solution is 50 g/L, 75 g/L, 100 g/L, 125 g/L, 150 g/L, 175 g/L, 200 g/L, 225 g/L, 250 g/L, 275 g/L, 300 g/L, 325 g/L, 350 g/L, 375 g/L, 400 g/L, 450 g/L, 500 g/L, 550 g/L or 600 g/L;
the method resulting in the negatively charged oligosaccharide with a concentration of at least 50 g/L in the final volume of the cultivation or incubation.

59. The method according to claim 54, wherein the cell produces 30 g/L or more of the negatively charged oligosaccharide in the whole broth and/or supernatant and/or wherein the negatively charged oligosaccharide in the whole broth and/or supernatant has a purity of at least 80% measured on the total amount of negatively charged oligosaccharide and its precursor(s) produced by the cell in the whole broth and/or supernatant, respectively.

60. The method according to claim 54, wherein the cell produces a mixture of two or more oligosaccharides wherein at least one of the two or more oligosaccharides is the negatively charged oligosaccharide.

61. The method according to claim 54, wherein the cell produces a mixture of one or more disaccharide(s) and one or more oligosaccharide(s) wherein at least one of the one or more oligosaccharide(s) is the negatively charged oligosaccharide.

62. The method according to claim 54, wherein the cell produces a mixture comprising the negatively charged oligosaccharide and at least one non-charged (neutral) oligosaccharide.

63. The method according to claim 54, wherein the cell produces a mixture comprising a) the negatively charged oligosaccharide and b) a disaccharide and/or a non-charged (neutral) oligosaccharide.

64. The method according to claim 54, wherein the negatively charged oligosaccharide is recovered from the cultivation or incubation medium and/or the cell, and/or wherein the negatively charged oligosaccharide is purified.

65. The method according to claim 54, wherein the separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, affinity chromatography, ion exchange chromatography, hydrophobic interaction chromatography and/or gel filtration, ligand exchange chromatography, and electrodialysis.

66. The method according to claim 54, wherein the purification comprises at least one of the following steps: utilizing activated charcoal or carbon, use of charcoal, nanofiltration, ultrafiltration, electrophoresis, enzymatic treatment or ion exchange, temperature adjustment, pH adjustment, pH adjustment with an alkaline or acidic solution, utilizing alcohols, utilizing aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.
